(19) United States
(12) Patent Application Publication
(10) Pub. No.: US 2010/0029598 A1
(43) Pub. Date: Feb. 4, 2010

(54) Extended Benzamide Derivatives as Modulators of the EP2 Receptor

(75) Inventors: Marcus Kopitz, Berlin (DE); Bernhard Lindenthal, Berlin (DE); Antonius Ter Laak, Berlin (DE); Tim Wintermantel, Koeln (DE); Olaf Peters, Tabarz (DE); Gernot Langer, Falkensee (DE); Nico Braeuer, Falkensee (DE); Bernd Buchmann, Hohen Neuendorf (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Bayer Schering Pharma AG, Berlin (DE)

(21) Appl. No.: 12/508,818

(22) Filed: Jul. 24, 2009

Related U.S. Application Data
(60) Provisional application No. 61/084,725, filed on Jul. 30, 2008.

(30) Foreign Application Priority Data
Jul. 30, 2008 (EP) 08161438.0

(51) Int. Cl. A61K 31/60; C07D 209/14; A61K 31/404; C07D 401/02; A61K 31/4439; C07D 403/02; C07D 241/36; A61K 31/498; A61K 31/501; A61K 31/506; A61K 31/5415; A61P 11/00; A61P 1/00; A61P 25/00; A61P 35/00

(52) U.S. Cl. 514/165; 548/506; 514/419; 548/455; 514/414; 546/277.4; 514/339; 548/261; 544/355; 514/249; 544/238; 514/252.06; 544/333; 514/256; 514/224.2

(57) ABSTRACT

The present invention relates to extended benzamide derivatives of the general formula I, to processes for preparation thereof and to use thereof for production of pharmaceutical compositions for treatment of disorders and indications associated with the EP₂ receptor.

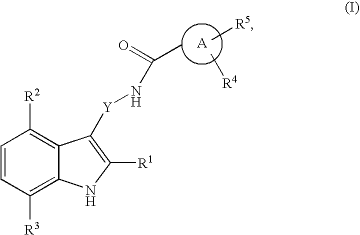

This application claims the benefit of the filing date of U.S. Provisional Application Ser. No. 61/084,725 filed Jul. 30, 2008, which is incorporated by reference herein.

The present invention relates to extended benzamide derivatives as EP₂ receptor modulators, to processes for preparation thereof and to use thereof as medicaments.

It has long been known that prostaglandins are key molecules in the processes of female reproductive biology, for example control of ovulation, of fertilization, of nidation, of decidualization (e.g. placenta formation) and of menstruation. Prostaglandins likewise play an important part in the pathological changes in the reproductive tract, including menorrhagia, dysmenorrhoea, endometriosis and cancer. The mechanism by which prostaglandins bring about these changes has not yet been completely elucidated. Recent results indicate that prostaglandins, their receptors and signal transduction pathways thereof are involved in processes such as angiogenesis, apoptosis, proliferation, and in inflammatory/antiinflammatory and immunological processes.

The effects of prostaglandins are mediated by their G protein-coupled receptors which are located on the cell surface. Prostaglandin E₂ (PGE₂) is of particular interest, having a wide variety of cellular effects through binding to functionally different receptor subtypes, namely the EP₁, EP₂, EP₃ and EP₄ receptors. The receptor subtypes to which prostaglandin E₂ binds appear to be of particular interest for the receptor-mediated effects which are involved in the control of fertility. It has thus been possible to show that the reproductive functions in EP₂ knockout mice (EP₂^{-/-}), i.e. in mice no longer having a functional PGE₂ receptor of the EP₂ subtype, are impaired, and that these animals have a smaller “litter size” (Matsumoto et al., 2001, Biology of Reproduction 64, 1557-1565). It was likewise possible to show that these EP₂ knockout mice (Hizaki et al. Proc Natl Acad Sci U.S.A. Aug. 31, 1999; 96(18):10501-10506) show distinctly reduced cumulus expansion and severe subfertility, which is to be regarded as causally connected with diminished reproductive processes such as ovulation and fertilization.

The EP₂ receptor accordingly represents an important target for developing medicaments for controlling female fertility. The existence of the 4 subclasses of the PGE₂ receptor opens up the possibility of targeted development of selectively active compounds. However, to date, scarcely any selective EP₂ receptor ligands which bind to the EP₂ subtypes of the PGE₂ receptor are known, since most known compounds also bind to the other PGE₂ receptor lo subtypes, for example to the EP₄ receptor.

EP₂ receptor antagonists are described, for example in the applications US2005059742 and EP1467738 (Jabbour, Medical Research Council). A method in which an EP₂ and/or an EP₄ antagonist can be employed for the treatment of menorrhagia and dysmenorrhoea is claimed. AH6809 is disclosed as antagonist of the EP₂ or EP₄ receptor, but no other specific antagonists and no new compounds are disclosed.

Ono Pharmaceutical claims in the application WO03/016254 the preparation of benzene acid or saturated carboxylic acid derivatives which are substituted by aryl or heterocycles, inter alia as PGE₂ receptor antagonists. The disclosed compounds are claimed for the treatment of a large number of disorders, including allergic disorders, Alzheimer's disease, pain, abortion, painful menstruation, menorrhagia and dysmenorrhoea, endometriosis, bone disorders, ischaemia etc. The described compounds are, however, distinguished by a particularly high affinity for the EP₃ receptor. A further application (WO04/032964) describes novel compounds which are likewise distinguished by a particularly high affinity for the EP₃ receptor, but also have EP₂-antagonistic effects and which are used for the treatment and prophylaxis of allergic disorders.

The application WO04/39807 of Merck Frosst, Canada, discloses the preparation of pyridopyrrolizines and pyridoindolizines. However, these compounds are distinguished by good binding to the PGD₂ receptor, and this receptor represents a different subtype of the prostaglandin receptor.

Naphthalene derivatives as EP₄ receptor ligands are disclosed in application US2004102508 of SmithKline Beecham Corporation. The claimed compounds are used for the treatment or prophylaxis of pain, allergic reactions and neurodegenerative disorders.

EP₄ antagonists (γ-lactams) are claimed in the application WO03/103604 (Applied Research Systems). The compounds bind approximately 60-fold better to the EP₄ than to the EP₂ receptor and are claimed inter alia for the treatment of premature labour, dysmenorrhoea, asthma, infertility or fertility impairments. The same company claims in the applications WO03/053923 (substituted pyrrolidines) or WO03/035064 (substituted pyrazolidinones) compounds for the treatment of disorders associated with prostaglandins, for example infertility, hypertension and osteoporosis. The compounds bind to the EP₄ and to the EP₂ receptor subtypes. The application WO03/037433 claims ω-cycloalkyl, 17 heteroaryl prostaglandin derivatives as EP₂ receptor antagonists, in particular for the treatment of elevated intraocular pressure.

The application WO03/064391 (Pfizer Products) describes metabolites of [3-[[N-(4-tert-butylbenzyl)(pyridin-3-ylsulphonyl)amino]methyl]acetic acid which inhibit the binding of [³H] prostaglandin E₂ to the EP₂ receptor. The use of these metabolites for the treatment of osteoporosis is disclosed.

Tani et al. claim in the application US2005124577 8-azaprostaglandin derivatives for the treatment of immunological disorders, allergic disorders, premature labour, abortion, etc. The compounds bind to the EP₂ and to the EP₄ receptor.

European patent application EP 1306087 describes EP₂ receptor agonists which are used for the treatment of erectile dysfunction (Ono Pharmaceuticals). The same class of structures is described in European patent EP 860430 (Ono Pharmaceuticals), and their use for the manufacture of a medicament for the treatment of immunological disorders, asthma and abortion is claimed. WO04/009117 describes EP₂ and EP₄ receptor agonists for the treatment of disorders caused by uterine contraction, for example painful menstruation (Ono Pharmaceuticals).

The applications WO003/74483 and WO03/09872 describe agonists which bind equally to the EP₂ and to the EP₄ receptor (Ono Pharmaceuticals).

Agonists of the EP₂ and of the EP₄ receptors are frequently described in connection with the treatment of osteoporosis (WO99/19300 (Pfizer), US2003/0166631 (Dumont Francis), WO03/77910 (Pfizer), WO03/45371 (Pfizer), WO03/74483 and WO03/09872 (Ono Pharmaceuticals)) and for glaucoma treatment (WO04/37813, WO04/37786, WO04/19938, WO03/103772, WO03/103664, WO03/40123, WO03/47513, WO03/47417 (Merck Frosst Canada)) and U.S. Pat. No. 6,410,591 and U.S. Pat. No. 6,747,037 (Allergan).

Applications WO05/035514 (Vertex) and JP2007045752 (Takeda) disclose indolylamines, but not as ligands of the EP₂ receptor. Application WO05/035514 describes the substances as modulators of ATP binding cassette transporters.

The patent application WO04/12656 (Applied Research Systems) claims EP₂ receptor agonists in connection with inflammation.

The patent application WO03/77919 (Merck & Co. Inc.) claims EP₄ receptor agonists for the treatment of fertility.

However, there are no known selective EP₂ receptor agonists and antagonists which regulate the processes which are ultimately responsible for ovulation, fertilization, nidation and decidualization, and which thus contribute to promotion or inhibition of fertility.

Patent applications to Bayer Schering Pharma AG (WO2007/057232, WO2007/071456, WO2008/028689, WO2008/028690 WO2008/028691) for the first time claimed selective antagonists of the EP₂ receptor. However, the compounds claimed bind only with a binding affinity in the micromolar range.

Therefore, only weakly effective EP₂ receptor agonists and antagonists are known. For the regulation of the processes ultimately responsible for ovulation, fertilization, nidation and decidualization and which thus contribute to promotion or inhibition of fertility, however, compounds which bind selectively to the EP₂ receptor with a high binding affinity are required.

It was therefore an object of the present invention to provide more potent, selective antagonists of the EP₂ receptor.

This object was achieved by the provision of the compounds of the general formula I

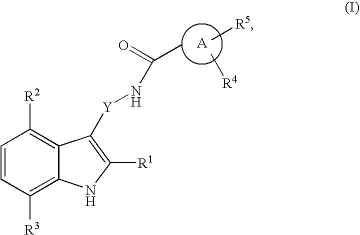

in which
A is a C₆-C₁₂-aryl or C₅-C₁₂-heteroaryl radical which may optionally be mono- or polysubstituted by R⁴ and/or R⁵,
R¹ is a C₁-C₆-alkyl radical which may optionally be substituted,
R² is a hydrogen, halogen, cyano, an —S(O)_{q}—CH₃ where q is 0-2, a C₁-C₄-alkoxy radical or C₁-C₆-alkyl, where this radical may be substituted as desired,
R³ is a hydrogen, a C₁-C₆-alkyl radical, cyano, chlorine or bromine,
R⁴ is a hydrogen, halogen, amino, an —S(O)ₚ—C₁-C₆-alkyl group where p is 0-2,
   a C₁-C₆-acyl, NH—CO—NH₂, —O—CO—NH(C₁-C₆-alkyl), —O—CO—N(C₁-C₆-alkyl)₂ or NH—CO—C₁-C₆-alkyl radical,
   a C₁-C₆-alkyl which may optionally be mono- or polysubstituted, identically or differently, by C₁-C₆-acyl, C₁-C₆-alkoxy, hydroxyl, cyano, CO₂—(C₁-C₆-alkyl), N—(C₁-C₆-alkyl)₂, C₅-C₁₂-heteroaryl, COOH, CO—NH₂, CO—NH(C₁-C₆-alkyl) or by CO—N(C₁-C₆-alkyl)₂,
   a C₁-C₆-alkoxy which may optionally be mono- or polysubstituted, identically or differently, by hydroxyl, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or by CO—N(C₁-C₆-alkyl)₂,
   an O—C₆-C₁₂-aryl which may optionally be substituted by hydroxyl, cyano, COOH or CO—NH₂,
   a CH₂O—C₆-C₁₂-aryl which may optionally be substituted by hydroxyl, cyano, COOH or CO—NH₂,
   an O—C₅-C₁₆-heteroaryl which may optionally be substituted by hydroxyl, cyano, COOH or CO—NH₂,
   a hydroxyl, cyano, O—CO—(C₁-C₆-alkyl), CO—NH(C₅-C₁₂-heteroaryl), NH—(C₁-C₆-alkyl), N—(C₁-C₆-alkyl)₂,
   a C₆-C₁₂-aryl which may optionally be mono- or polysubstituted, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxyl, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C6-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or by —S(O)ᵣ—CH₃ where r is 0-2, or two adjacent positions may be substituted by —O—CH₂—O— or —O—C(CH₃)₂—O—,
   a monocyclic C₅-C₇-heteroaryl which may optionally be mono- or polysubstituted, identically or differently, by C₁-C₆-alkyl,
      provided that R² is cyano or R¹ and/or R² are the same or different and are each a C₁-C₆-alkyl radical, where at least one of the radicals is at least monosubstituted or
      provided that R⁵ is an —S(O)ₚ—C₁-C₆-alkyl where p is 0-2, is a C₁-C₆-acyl, —O—CO—NH(C₁-C₆-alkyl), —O—CO—N(C₁-C₆-alkyl)₂, C₆-C₁₂-aryloxy, C₅-C₁₆-heteroaryloxy, hydroxyl, cyano or N—(C₁-C₆-alkyl)₂,
   a monocyclic C₅-C₇-heteroaryl which may be at least mono- or polysubstituted, identically or differently, by halogen, CF₃, C₁-C₆-acyl, C₁-C₆-alkoxy, hydroxyl, CH₂—OH, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂,
   a bi- or tricyclic C₈-C₁₂-heteroaryl which may optionally be mono- or polysubstituted, identically or differently, by halogen, by C₁-C₆-alkyl, C₁-C₆-acyl, C₁-C₆-alkoxy, hydroxyl, cyano, CO₂-(C₁-C₆-alkyl), N—(C₁-C₆-alkyl)₂, COOH, CO—NH₂, CO—NH(C₁-C₆-alkyl) or CO—N(C₁-C₆-alkyl)₂ or
   a C₃-C₆-cycloalkyl which may optionally be mono- or polysubstituted, identically or differently, by halogen, by C₁-C₆-alkyl, hydroxyl, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R⁵ is a hydrogen, halogen, amino, —S(O)ₚ—C₁-C₆-alkyl where p is 0-2,
   a C₁-C₆-acyl, NH—CO—NH₂, NH—CO—C₁-C₆-alkyl, —O—CO—NH(C₁-C₆-alkyl), —O—CO—N(C₁-C₆-alkyl)₂ or C₁-C₆-alkyl group which may optionally be mono- or polysubstituted, identically or differently, by C₁-C₆-acyl, C₁-C₆-alkoxy, hydroxyl, cyano, CO₂-(C₁-C₆-alkyl), N—(C₁-C₆-alkyl)₂, C₅-C₁₂-heteroaryl, COOH, CO—NH₂, CO—NH(C₁-C₆-alkyl) or by CO—N(C₁-C₆-alkyl)₂,
   a C₁-C₆-alkoxy which may optionally be mono- or polysubstituted, identically or differently, by hydroxyl, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or by CO—N(C₁-C₆-alkyl)₂,
   an O—C₆-C₁₂-aryl which may optionally be substituted by hydroxyl, cyano, COOH or CO—NH₂,
   a CH₂O—C₆-C₁₂-aryl which may optionally be substituted by hydroxyl, cyano, COOH or CO—NH₂,
   an O—C₅-C₁₆-heteroaryl which may optionally be substituted by hydroxyl, cyano, COOH or CO—NH₂,
   a hydroxyl, cyano, O—CO—(C₁-C₆-alkyl), CO—NH(C₅-C₁₂-heteroaryl), NH—(C₁-C₆-alkyl), N—(C₁-C₆-alkyl)₂,
   a C₆-C₁₂-aryl which may optionally be mono- or polysubstituted, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxyl, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or by —S(O)ᵣ—CH₃ where r is 0-2, or two adjacent positions may be substituted by —O—CH₂—O— or —O—C(CH₃)₂—O—,
   a monocyclic C₅-C₇-heteroaryl which may optionally be mono- or polysubstituted, identically or differently, by C₁-C₆-alkyl when R² is cyano, or
      provided that R¹ and/or R² are the same or different and are each a C₁-C₆-alkyl radical, where at least one of the radicals is at least monosubstituted,
   a monocyclic C₅-C₇-heteroaryl which may be at least mono- or polysubstituted, identically or differently, by halogen, CF₃, C₁-C₆-acyl, C₁-C₆-alkoxy, hydroxyl, CH₂—OH, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂,
   a bi- or tricyclic C₈-C₁₂-heteroaryl which may optionally be mono- or polysubstituted, identically or differently, by halogen, by C₁-C₆-alkyl, C₁-C₆-acyl, C₁-C₆-alkoxy, hydroxyl, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂ or
   a C₃-C₆-cycloalkyl which may optionally be mono- or polysubstituted, identically or differently, by halogen, by C₁-C₆-alkyl, hydroxyl, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R⁴ and R⁵ are in ortho, meta or meta,para positions with respect to one another and together are defined as —O—CO—S—, —S—CO—O—, CH₂—CO—O—, O—CO—CH₂—, —CH₂—CO—NH—, —NH—CO—CH₂—, —O—O—NH—, —NH—CO—O—, —CO—CH₂—(CH₂)ₘ—, —CH₂—(CH₂)ₘCO—, —O—(CH₂)ₘ—O—, —O—C—(CH₃)₂—O—, —CH₂—(CH₂)ₘ—CH₂— where m is 1-3,
Y is a —(CH₂), group where n is 1-3,

and the isomers, diastereomers, enantiomers and salts or cyclodextrin clathrates thereof, which overcome the known disadvantages and have improved properties, i.e. bind to the EP₂ receptor with high binding affinity, and have good efficacy, good solubility and stability, excluding the following compounds:

4-chloro-1,3-dimethyl-N-[2-(2-methyl-1H-indol-3-yl)ethyl]-1H-pyrazole-5-carboxamide

N-[2-(2-methyl-1H-indol-3-yl)ethyl]-6-quinoxalinecarboxamide

3,5-dimethyl-N-[2-(2-methyl-1H-indol-3-yl)ethyl]-4-isoxazolecarboxamide

5,7-dimethyl-N-[2-(2-methyl-1H-indol-3-yl)ethyl]pyrazolo[1,5-a]pyrimidine-2-carboxamide

N-[2-(2-methyl-1H-indol-3-yl)ethyl]-2-methylbenzamide

N-[2-(2-methyl-1H-indol-3-yl)ethyl]-2-(difluoromethyl)benzamide

3-iodo-1-methyl-N-[2-(2-methyl-1H-indol-3-yl)ethyl]-1H-pyrrole-2-carboxamide

1-methyl-N-[2-(2-methyl-1H-indol-3-yl)ethyl]-4-(trifluoromethyl)-1H-pyrrole-3-carboxamide

2-iodo-N-[2-(2-methyl-1H-indol-3-yl)ethyl]-3-thiophenecarboxamide

3-(difluoromethyl)-1-methyl-N-[2-(2-methyl-1H-indol-3-yl)ethyl]-1H-pyrazole-4-carboxamide

3-(trifluoromethyl)-1-methyl-N-[2-(2-methyl-1H-indol-3-yl)ethyl]-1H-pyrazole-4-carboxamide

5-fluoro-1,3-dimethyl-N-[2-(2-methyl-1H-indol-3-yl)ethyl]-1H-pyrazole-4-carboxamide

4-(difluoromethyl)-2-methyl-N-[2-(2-methyl-1H-indol-3-yl)ethyl]-5-thiazolecarboxamide

4-(trifluoromethyl)-2-methyl-N-[2-(2-methyl-1H-indol-3-yl)ethyl]-5-thiazolecarboxamide

2-iodo-N-[2-(2-methyl-1H-indol-3-yl)ethyl]-3-furancarboxamide

2,5-dimethyl-N-[2-(2-methyl-1H-indol-3-yl)ethyl]-3-furancarboxamide

5-methyl-N-[2-(2-methyl-1H-indol-3-yl)ethyl]-2-(trifluoromethyl)-3-furancarboxamide

2-methyl-N-[2-(2-methyl-1H-indol-3-yl)ethyl]-3-furancarboxamide

3-methyl-N-[2-(2-methyl-1H-indol-3-yl)ethyl]-2-thiophenecarboxamide

3-iodo-N-[2-(2-methyl-1H-indol-3-yl)ethyl]-2-thiophenecarboxamide

3-bromo-N-[2-(2-methyl-1H-indol-3-yl)ethyl]-2-thiophenecarboxamide

4-methoxy-N-[2-(2-methyl-1H-indol-3-yl)ethyl]benzamide

N-[2-(7-chloro-2-methyl-1H-indol-3-yl)ethyl]-2-methyl-7-benzoxazolecarboxamide

4-(1,1-dimethylethyl)-N-[2-(2-methyl-1H-indol-3-yl)ethyl]benzamide

N-[2-(2,7-dimethyl-1H-indol-3-yl)ethyl]-3,4-dimethoxybenzamide

2-ethoxy-N-[2-(2-methyl-1H-indol-3-yl)ethyl]benzamide

2-(1-methylethoxy)-N-[2-(2-methyl-1H-indol-3-yl)ethyl]benzamide

2-chloro-4,5-difluoro-N-[2-(2-methyl-1H-indol-3-yl)ethyl]benzamide

4-bromo-N-[2-(4-fluoro-2,7-dimethyl-1H-indol-3-yl)ethyl]benzamide

2,6-difluoro-N-[2-(2-methyl-1H-indol-3-yl)ethyl]benzamide

3,4-difluoro-N-[2-(2-methyl-1H-indol-3-yl)ethyl]benzamide

N-[2-(2-methyl-1H-indol-3-yl)ethyl]-4-(trifluoromethyl)benzamide

N-[2-(2-methyl-1H-indol-3-yl)ethyl]-4-methylbenzamide

2,4-dichloro-N-[2-(2-methyl-1H-indol-3-yl)ethyl]benzamide

2-bromo-N-[2-(2-methyl-1H-indol-3-yl)ethyl]benzamide

4-fluoro-N-[2-(2-methyl-1H-indol-3-yl)ethyl]benzamide

N-[2-(4-fluoro-2,7-dimethyl-1H-indol-3-yl)ethyl]-2-methylbenzamide

N-[2-(4-fluoro-2,7-dimethyl-1H-indol-3-yl)ethyl]-2-methoxybenzamide

3,4-dimethoxy-N-[2-(4-fluoro-2,7-dimethyl-1H-indol-3-yl)ethyl]benzamide

4-[[[2-(4-fluoro-2,7-dimethyl-1H-indol-3-yl)ethyl]amino]carbonyl]benzoic acid methyl ester

N-[2-(4-fluoro-2,7-dimethyl-1H-indol-3-yl)ethyl]-1-methyl-1H-pyrazole-5-carboxamide

N-[2-(7-ethyl-2-methyl-1H-indol-3-yl)ethyl]-2-methoxybenzamide

4-chloro-N-[2-(2-methyl-1H-indol-3-yl)ethyl]benzamide

2,6-dichloro-N-[2-(2-methyl-1H-indol-3-yl)ethyl]benzamide

5-chloro-2-methoxy-N-[2-(2-methyl-1H-indol-3-yl)ethyl]benzamide

3,4-dichloro-N-[2-(2-methyl-1H-indol-3-yl)ethyl]benzamide

2-fluoro-N-[2-(2-methyl-1H-indol-3-yl)ethyl]benzamide

4-chloro-1-methyl-N-[2-(2-methyl-1H-indol-3-yl)ethyl]-1H-pyrazole-5-carboxamide

4-[[[2-(2-methyl-1H-indol-3-yl)ethyl]amino]carbonyl]benzoic acid methyl ester

N-[2-(2-methyl-1H-indol-3-yl)ethyl]-2-thiophenecarboxamide

3,4,5-trimethoxy-N-[2-(2-methyl-1H-indol-3-yl)ethyl]benzamide

3-methoxy-N-[2-(2-methyl-1H-indol-3-yl)ethyl]benzamide

N-[2-(2-methyl-1H-indol-3-yl)ethyl]-4-pyridinecarboxamide

N-[2-(2-methyl-1H-indol-3-yl)ethyl]-3-pyridinecarboxamide

The inventive compounds have an antagonistic effect on the EP₂ receptor and therefore serve for female fertility control.

C₁-C₄-Alkyl or C₁-C₆-alkyl is in each case understood to mean a straight-chain or branched alkyl radical, for example methyl, ethyl, propyl, isopropyl, butyl, isobutyl, sec-butyl, tert-butyl, pentyl, isopentyl and hexyl.

The alkyl radicals may optionally be mono- or polysubstituted, identically or differently, by halogen.

C₁-C₄-Alkoxy or C₁-C₆-alkoxy is in each case understood to mean a straight-chain or branched alkoxy radical, for example methoxy, ethoxy, n-propoxy, isopropoxy, n-butoxy, sec-butoxy, isobutoxy, tert-butyloxy, pentoxy, isopentoxy and hexoxy.

The alkoxy radicals may optionally be mono- or polysubstituted, identically or differently, by halogen.

C₁-C₄-Acyl or C₁-C₆-acyl is in each case understood to mean a straight-chain or branched radical, for example formyl, acetyl, propionyl, butyroyl, isobutyroyl, valeroyl and benzoyl.

The acyl radicals may optionally be mono- or polysubstituted, identically or differently, by halogen.

C₃-C₆-Cycloalkyl is understood to mean monocyclic alkyl rings such as cyclopropyl, cyclobutyl, cyclopentyl and cyclohexyl.

The cycloalkyl radicals may, instead of the carbon atoms, contain one or more heteroatoms such as oxygen, sulphur and/or nitrogen. Preferred heterocycloalkyls are those having 3 to 6 ring atoms, for example aziridinyl, azetidinyl, pyrrolidinyl, piperidinyl, piperazinyl, morpholinyl. Ring systems in which one or more possible double bonds may optionally be present in the ring are, for example, cycloalkenyls such as cyclopropenyl, cyclobutenyl, cyclopentenyl, cyclopentadienyl, cyclohexenyl, which may be attached either at the double bond or at the single bonds.

Halogen is in each case understood to mean fluorine, chlorine, bromine or iodine.

The C₆-C₁₂-aryl radical includes in each case 6-12 carbon atoms and may, for example, be benzofused. Examples include: phenyl, tropyl, cyclooctadienyl, indenyl, naphthyl, biphenyl, fluorenyl, anthracenyl etc.

A corresponding aryloxy radical includes an aryl radical joined via an oxygen bridge.

The monocyclic C₅-C₇-heteroaryl radical, the bicyclic and tricyclic C₈-C₁₂-heteroaryl radical and the C₅-C₁₂- or C₅-C₁₆-heteroaryl radical are understood to mean ring systems which contain in each case 5-16 ring atoms and which may, instead of the carbon, contain one or more, identical or different, heteroatoms such as oxygen, sulphur or nitrogen, and where the C₈-C₁₂- and the C₅-C₁₆-heteroaryl radical may be mono-, bi- or tricyclic and may additionally in each case be benzofused.

Examples include:

thienyl, furanyl, pyrrolyl, oxazolyl, imidazolyl, pyrazolyl, isoxazolyl, isothiazolyl, oxadiazolyl, triazolyl, thiadiazolyl, etc. and benzo derivatives thereof, for example benzofuranyl, benzothienyl, benzoxazolyl, benzimidazolyl, indazolyl, indolyl, isoindolyl, etc; or pyridinyl, pyridazinyl, pyrimidinyl, pyrazinyl, triazinyl, etc. and benzo derivatives thereof, for example quinolyl, isoquinolyl, etc; or azocinyl, indolizinyl, purinyl, etc. and benzo derivatives thereof; or quinolinyl, isoquinolinyl, cinnolinyl, phthalazinyl, quinazolinyl, quinoxalinyl, naphthyridinyl, pteridinyl, carbazolyl, acridinyl, phenazinyl, phenothiazinyl, phenoxazinyl, xanthenyl, oxepinyl, benzotriazole, etc.

The heteroaryl radical may in each case be benzofused. Examples of 5-membered heteroaromatic rings include: thiophene, furan, oxazole, thiazole, imidazole, pyrazole and benzo derivatives thereof, and examples of 6-membered heteroaromatic rings include pyridine, pyrimidine, triazine, quinoline, isoquinoline and benzo derivatives.

Heteroatoms are understood to mean oxygen, nitrogen or sulphur atoms. A corresponding heteroaryloxy radical includes a heteroaryl radical joined via an oxygen bridge.

If an acidic function is present, suitable salts are the physiologically tolerated salts of organic and inorganic bases, for example the readily soluble alkali metal and alkaline earth metal salts, and N-methylglucamine, dimethylglucamine, ethylglucamine, lysine, 1,6-hexadiamine, ethanolamine, glucosamine, sarcosine, serinol, tris-hydroxymethylaminomethane, aminopropanediol, Sovak base, 1-amino-2,3,4-butanetriol.

If a basic function is present, the physiologically tolerated salts of organic and inorganic acids are suitable, such as hydrochloric acid, sulphuric acid, phosphoric acid, citric acid, tartaric acid, among others.

Preference is given to those compounds of the general formula (I) where
A is a C₆-C₁₂-aryl or C₅-C₁₂-heteroaryl radical which may optionally be mono- or polysubstituted by R⁴ and/or R⁵,
R¹ is a C₁-C₄-alkyl radical which may optionally be substituted,
R² is a hydrogen, halogen, a C₁-C₄-alkoxy radical or C₁-C₆-alkyl, where this radical may be substituted as desired,
R³ is a hydrogen, a C₁-C₆-alkyl radical, cyano, chlorine or bromine, and the R⁴, R⁵, R⁶, R⁷ and Y radicals are each as defined above,

and the isomers, diastereomers, enantiomers and salts or cyclodextrin clathrates thereof.

Preference is likewise given to those compounds of the general formula (I) where
A is a C₆-C₁₂-aryl or C₅-C₁₂-heteroaryl radical which may optionally be mono- or polysubstituted by R⁴ and/or R⁵,
R¹ is a C₁-C₄-alkyl radical,
R² is a hydrogen, halogen or C₁-C₄-alkyl, where this radical may be substituted as desired,
R³ may be a hydrogen, a C₁-C₄-alkyl radical, cyano, chlorine or bromine,
R⁴ is a hydrogen, halogen, amino, an —S(O)ₚ—C₁-C₄-alkyl group where p is 0-2,
   a C₁-C₄-acyl, NH—CO—NH₂, —O—CO—NH(C₁-C₄-alkyl), —O—CO—N(C₁-C₄-alkyl)₂ or NH—CO—C₁-C₄-alkyl radical,
   a C₁-C₄-alkyl which may optionally be mono- or polysubstituted, identically or differently, by C₁-C₄-acyl, C₁-C₄-alkoxy, hydroxyl, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
   a C₁-C₄-alkoxy which may optionally be mono- or polysubstituted, identically or differently, by hydroxyl, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
   an O—C₆-C₁₂-aryl which may optionally be substituted by hydroxyl, cyano, COOH or CO—NH₂,
   a CH₂O—C₆-C₁₂-aryl which may optionally be substituted by hydroxyl, cyano, COOH or CO—NH₂,
   an O—C₅-C₁₆-heteroaryl which may optionally be substituted by hydroxyl, cyano, COOH or CO—NH₂,
   a hydroxyl, cyano, O—CO—(C₁-C₄-alkyl), CO—NH(C₅-C₁₂-heteroaryl), NH—(C₁-C₄-alkyl), N—(C₁-C₄-alkyl)₂,
   a C₆-C₁₂-aryl which may optionally be mono- or polysubstituted, identically or differently, by halogen, by C₁-C₄-alkyl, C₃-C₆-cycloalkyl, C₁-C₄-acyl, C₁-C₄-alkoxy, C₆-C₁₂-aryl, C₅-C₁₂-heteroaryl, hydroxyl, CH₂—OH, cyano, CH₂—CN, amino, CO₂—(C₁-C₄-alkyl), N—(C₁-C₄-alkyl)₂, NHSO₂CH₃, SO₂NH₂, SO₂NH(C₁-C₄-alkyl), SO₂N(C₁-C₄-alkyl)₂, COOH, CO—NH₂, CO—NH(C₁-C₄-alkyl), CO—N(C₁-C₄-alkyl)₂, CO—NH(C₅-C₁₂-heteroaryl), NH—CO(C₁-C₄-alkyl), CH₂—NH—CO(C₁-C₄-alkyl), NH—CO(C₅-C₁₂-heteroaryl), CH₂—NH—CO(C₅-C₁₂-heteroaryl), styryl, or by —S(O)ᵣ—CH₃ where r is 0-2, or two adjacent positions may be substituted by —O—CH₂—O— or —O—C(CH₃)₂—O—,
   a monocyclic C₅-C₇-heteroaryl which may optionally be mono- or polysubstituted, identically or differently, by C₁-C₄-alkyl,
   provided that R² is cyano or R¹ and/or R² are the same or different and are each a C₁-C₄-alkyl radical, where at least one of the radicals is at least monosubstituted or
   provided that R⁵ is an —SO(O)ₚ—C₁-C₄-alkyl where p is 0-2, is a C₁-C₆-acyl, —O—CO—NH (C₁-C₄-alkyl), —O—CO—N(C₁-C₄-alkyl)₂, C₆-C₁₂-aryloxy, C₅-C₁₆-heteroaryloxy, hydroxyl, cyano or N—(C₁-C₄-alkyl)₂,
   a monocyclic C₅-C₇-heteroaryl which may be at least mono- or polysubstituted, identically or differently, by halogen, CF₃, C₁-C₄-acyl, C₁-C₄-alkoxy, hydroxyl, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂,
   a bi- or tricyclic C₈-C₁₂-heteroaryl which may optionally be mono- or polysubstituted, identically or differently, by halogen, by C₁-C₄-alkyl, C₁-C₄-acyl, C₁-C₄-alkoxy, hydroxyl, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂ or
   a C₃-C₆-cycloalkyl which may optionally be mono- or polysubstituted, identically or differently, by halogen, by C₁-C₄-alkyl, hydroxyl, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R⁵ is a hydrogen, halogen, amino, —SO(O)ₚ—C₁-C₄-alkyl where p is 0-2,
   a C₁-C₄-acyl, NH—CO—NH₂—, NH—CO—C₁-C₄-alkyl-, —O—CO—NH(C₁-C₄-alkyl)-, —O—CO—N(C₁-C₄-alkyl)₂ or C₁-C₄-alkyl group which may optionally be mono- or polysubstituted, identically or differently, by C₁-C₄-acyl, C₁-C₄-alkoxy, hydroxyl, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
   a C₁-C₄-alkoxy which may optionally be mono- or polysubstituted, identically or differently, by hydroxyl, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
   an O—C₆-C₁₂-aryl which may optionally be substituted by hydroxyl, cyano, COOH or CO—NH₂,
   a CH₂O—C₆-C₁₂-aryl which may optionally be substituted by hydroxyl, cyano, COOH or CO—NH₂,
   an O—C₅-C₁₆-heteroaryl which may optionally be substituted by hydroxyl, cyano, COOH or CO—NH₂,
   a hydroxyl, cyano, O—CO—(C₁-C₄-alkyl), CO—NH(C₅-C₁₂-heteroaryl), NH—(C₁-C₄-alkyl), N—(C₁-C₄-alkyl)₂,
   a C₆-C₁₂-aryl which may optionally be mono- or polysubstituted, identically or differently, by halogen, by C₁-C₄-alkyl, C₃-C₆-cycloalkyl, C₁-C₄-acyl, C₁-C₄-alkoxy, C₆-C₁₂-aryl, C₅-C₁₂-heteroaryl, hydroxyl, CH₂—OH, cyano, CH₂—CN, amino, CO₂—(C₁-C₄-alkyl), N—(C₁-C₄-alkyl)₂, NHSO₂CH₃, SO₂NH₂, SO₂NH(C₁-C₄-alkyl), SO₂N(C₁-C₄-alkyl)₂, COOH, CO—NH₂, CO—NH(C₁-C₄-alkyl), CO—N(C₁-C₄-alkyl)₂, CO—NH(C₅-C₁₂-heteroaryl), NH—CO(C₁-C₄-alkyl), CH₂—NH—CO(C₁-C₄-alkyl), NH—CO(C₅-C₁₂-heteroaryl), CH₂—NH—CO(C₅-C₁₂-heteroaryl), styryl, or an —S(O)ᵣ—CH₃ where r is 0-2, or two adjacent positions may be substituted by —O—CH₂—O— or —O—C(CH₃)₂—O—,
   a monocyclic C₅-C₇-heteroaryl which may optionally be mono- or polysubstituted, identically or differently, by C₁-C₄-alkyl, provided that R² is cyano or when R¹ and/or R² is the same or different and is a C₁-C₄-alkyl radical, where at least one of the radicals is at least monosubstituted,
   a monocyclic C₅-C₇-heteroaryl which may be at least mono- or polysubstituted, identically or differently, by halogen, CF₃, C₁-C₄-acyl, C₁-C₄-alkoxy, hydroxyl, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂,
   a bi- or tricyclic C₈-C₁₂-heteroaryl which may optionally be mono- or polysubstituted, identically or differently, by halogen, by C₁-C₄-alkyl, C₁-C₄-acyl, C₁-C₄-alkoxy, hydroxyl, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂ or
   a C₃-C₆-cycloalkyl which may optionally be mono- or polysubstituted, identically or differently, by halogen, by C₁-C₄-alkyl, hydroxyl, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R⁴ and R⁵ are in ortho, meta or meta,para positions with respect to one another and together are defined as —O—CO—S—, —S—CO—O—, —CH₂—CO—O—, —O—CO—CH₂—, —CH₂—CO—NH—, —NH—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂— where m is 1-3,
Y is a —(CH₂)ₙ group where n is 1-3,

and the isomers, diastereomers, enantiomers and salts or cyclodextrin clathrates thereof.

Preference is likewise given to those compounds of the general formula (I) where
A is a phenyl, naphthyl or heteroaryl radical which may optionally be mono- or disubstituted by R⁴ and/or R⁵,
R¹ is a C₁-C₄-alkyl radical,
R² is a hydrogen, halogen or C₁-C₄-alkyl, where this radical may be substituted as desired,
R³ is a hydrogen, a C₁-C₄-alkyl radical, cyano, chlorine or bromine,
R⁴ is a hydrogen, halogen, amino, an —SO(O)ₚ—C₁-C₄-alkyl group where p is 0-2,
   a C₁-C₄-acyl, NH—CO—NH₂, —O—CO—NH(C₁-C₄-alkyl), —O—CO—N(C₁-C₄-alkyl)₂ or NH—CO—C₁-C₄-alkyl radical,
   a C₁-C₄-alkyl which may optionally be mono- or polysubstituted, identically or differently, by C₁-C₄-acyl, C₁-C₄-alkoxy, hydroxyl, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
   a C₁-C₄-alkoxy which may optionally be mono- or polysubstituted, identically or differently, by hydroxyl, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
   an O—C₆-C₁₂-aryl which may optionally be substituted by hydroxyl, cyano, COOH or CO—NH₂,
   a CH₂O—C₆-C₁₂-aryl which may optionally be substituted by hydroxyl, cyano, COOH or CO—NH₂,
   an O—C₅-C₁₆-heteroaryl which may optionally be substituted by hydroxyl, cyano, COOH or CO—NH₂,
   a hydroxyl, cyano, O—CO—(C₁-C₄-alkyl), CO—NH(C₅-C₁₂-heteroaryl), NH—(C₁-C₄-alkyl), N—(C₁-C₄-alkyl)₂,
   a C₆-C₁₂-aryl which may optionally be mono- or polysubstituted, identically or differently, by halogen, by C₁-C₄-alkyl, C₃-C₆-cycloalkyl, C₁-C₄-acyl, C₁-C₄-alkoxy, C₆-C₁₂-aryl, C₅-C₁₂-heteroaryl, hydroxyl, CH₂—OH, cyano, CH₂—CN, amino, CO₂—(C₁-C₄-alkyl), N—(C₁-C₄-alkyl)₂, NHSO₂CH₃, SO₂NH₂, SO₂NH(C₁-C₄-alkyl), SO₂N(C₁-C₄-alkyl)₂, COOH, CO—NH₂, CO—NH(C₁-C₄-alkyl), CO—N(C₁-C₄-alkyl)₂, CO—NH(C₅-C₁₂-heteroaryl), NH—CO(C₁-C₄-alkyl), CH₂—NH—CO(C₁-C₄-alkyl), NH—CO(C₅-C₁₂-heteroaryl), CH₂—NH—CO(C₅-C₁₂-heteroaryl), styryl, or by —S(O)ᵣ—CH₃ where r is 0-2, or two adjacent positions may be substituted by —O—CH₂—O— or —O—C(CH₃)₂—O—,
   a monocyclic C₅-C₇-heteroaryl which may optionally be mono- or polysubstituted, identically or differently, by C₁-C₄-alkyl,
   provided that R² cyano or R¹ and/or R² are the same or different and are each a C₁-C₄-alkyl radical, where at least one of the radicals is at least monosubstituted or
   provided that R⁵ is an —SO(O)ₚ—C₁-C₄-alkyl where p is 0-2, is a C₁-C₆-acyl, —O—CO—NH(C₁-C₄-alkyl), —O—CO—N (C₁-C₄-alkyl)₂, C₆-C₁₂-aryloxy, C₅-C₁₆-heteroaryloxy, hydroxyl, cyano or N—(C₁-C₄-alkyl)₂,
   a monocyclic C₅-C₇-heteroaryl which may be at least mono- or polysubstituted, identically or differently, by halogen, CF₃, C₁-C₄-acyl, C₁-C₄-alkoxy, hydroxyl, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂,
   a bi- or tricyclic C₈-C₁₂-heteroaryl which may optionally be mono- or polysubstituted, identically or differently, by halogen, by C₁-C₄-alkyl, C₁-C₄-acyl, C₁-C₄-alkoxy, hydroxyl, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂ or
   a C₃-C₆-cycloalkyl which may optionally be mono- or polysubstituted, identically or differently, by halogen, by C₁-C₄-alkyl, hydroxyl, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R⁵ is a hydrogen, halogen, amino, —SO(O)ₚ—C₁-C₄-alkyl where p is 0-2,
   a C₁-C₄-acyl, NH—CO—NH₂—, NH—CO—C₁-C₄-alkyl-, —O—CO—NH(C₁-C₄-alkyl)-, —O—CO—N(C₁-C₄-alkyl)₂ or C₁-C₄-alkyl group which may optionally be mono- or polysubstituted, identically or differently, by C₁-C₄-acyl, C₁-C₄-alkoxy, hydroxyl, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
   a C₁-C₄-alkoxy which may optionally be mono- or polysubstituted, identically or differently, by hydroxyl, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
   an O—C₆-C₁₂-aryl which may optionally be substituted by hydroxyl, cyano, COOH or CO—NH₂,
   a CH₂O—C₆-C₁₂-aryl which may optionally be substituted by hydroxyl, cyano, COOH or CO—NH₂,
   an O—C₅-C₁₆-heteroaryl which may optionally be substituted by hydroxyl, cyano, COOH or CO—NH₂,
   a hydroxyl, cyano, O—CO—(C₁-C₄-alkyl), CO—NH(C₅-C₁₂-heteroaryl), NH—(C₁-C₄-alkyl), N—(C₁-C₄-alkyl)₂,
   a C₆-C₁₂-aryl which may optionally be mono- or polysubstituted, identically or differently, by halogen, by C₁-C₄-alkyl, C₃-C₆-cycloalkyl, C₁-C₄-acyl, C₁-C₄-alkoxy, C₆-C₁₂-aryl, C₅-C₁₂-heteroaryl, hydroxyl, CH₂—OH, cyano, CH₂—CN, amino, CO₂—(C₁-C₄-alkyl), N—(C₁-C₄-alkyl)₂, NHSO₂CH₃, SO₂NH₂, SO₂NH(C₁-C₄-alkyl), SO₂N(C₁-C₄-alkyl)₂, COOH, CO—NH₂, CO—NH(C₁-C₄-alkyl), CO—N(C₁-C₄-alkyl)₂, CO—NH(C₅-C₁₂-heteroaryl), NH—CO(C₁-C₄-alkyl), CH₂—NH—CO(C₁-C₄-alkyl), NH—CO(C₅-C₁₂-heteroaryl), CH₂—NH—CO(C₅-C₁₂-heteroaryl), styryl, or an —S(O)ᵣ—CH₃ where r is 0-2, or two adjacent positions may be substituted by —O—CH₂—O— or —O—C(CH₃)₂—O—,
   a monocyclic C₅-C₇-heteroaryl which may optionally be mono- or polysubstituted, identically or differently, by C₁-C₄-alkyl, provided that R² is cyano or when R¹ and/or R² are the same or different and are each a C₁-C₄-alkyl radical, where at least one of the radicals is at least monosubstituted,
   a monocyclic C₅-C₇-heteroaryl which may be at least mono- or polysubstituted, identically or differently, by halogen, CF₃, C₁-C₄-acyl, C₁-C₄-alkoxy, hydroxyl, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂,
   a bi- or tricyclic C₈-C₁₂-heteroaryl which may optionally be mono- or polysubstituted, identically or differently, by halogen, by C₁-C₄-alkyl, C₁-C₄-acyl, C₁-C₄-alkoxy, hydroxyl, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂ or
   a C₃-C₆-cycloalkyl which may optionally be mono- or polysubstituted, identically or differently, by halogen, by C₁-C₄-alkyl, hydroxyl, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R⁴and R⁵ are in ortho, meta or meta,para positions with respect to one another and together are defined as —O—CO—S—, —S—CO—O—, —CH₂—CO—O—, O—CO—CH₂—, —CH₂—CO—NH—, —NH—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂— where m is 1-3,
Y is a —(CH₂)₂ group,

and the isomers, diastereomers, enantiomers and salts or cyclodextrin clathrates thereof.

Preference is likewise given to those compounds of the general formula (I) where
A is a phenyl, naphthyl or C₅-C₁₂-heteroaryl radical which may optionally be mono- or disubstituted by R⁴ and/or R⁵,
R¹ is a C₁-C₄-alkyl radical,
R² is a hydrogen, fluorine, chlorine, bromine or C₁-C₄-alkyl,
R³ is a hydrogen, methyl, ethyl, trifluoromethyl, cyano, chlorine or bromine,
R⁴ is a hydrogen, halogen, amino, an —SO(O)ₚ—C₁-C₄-alkyl group where p is 0-2,
   a C₁-C₄-acyl, NH—CO—NH₂, —O—CO—NH(C₁-C₄-alkyl), —O—CO—N(C₁-C₄-alkyl)₂ or NH—CO—C₁-C₄-alkyl radical,
   a C₁-C₄-alkyl which may optionally be mono- or polysubstituted, identically or differently, by C₁-C₄-acyl, C₁-C₄-alkoxy, hydroxyl, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
   a C₁-C₄-alkoxy which may optionally be mono- or polysubstituted, identically or differently, by hydroxyl, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
   an O—C₆-C₁₂-aryl which may optionally be substituted by hydroxyl, cyano, COOH or CO—NH₂,
   a CH₂O—C₆-C₁₂-aryl which may optionally be substituted by hydroxyl, cyano, COOH or CO—NH₂,
   an O—C₅-C₁₆-heteroaryl which may optionally be substituted by hydroxyl, cyano, COOH or CO—NH₂,
   a hydroxyl, cyano, O—CO—(C₁-C₄-alkyl), CO—NH(C₅-C₁₂-heteroaryl), NH—(C₁-C₄-alkyl), N—(C₁-C₄-alkyl)₂,
   a C₆-C₁₂-aryl which may optionally be mono- or polysubstituted, identically or differently, by halogen, by C₁-C₄-alkyl, C₃-C₆-cycloalkyl, C₁-C₄-acyl, C₁-C₄-alkoxy, C₆-C₁₂-aryl, C₅-C₁₂-heteroaryl, hydroxyl, CH₂—OH, cyano, CH₂—CN, amino, CO₂—(C₁-C₄-alkyl), N—(C₁-C₄-alkyl)₂, NHSO₂CH₃, SO₂NH₂, SO₂NH(C₁-C₄-alkyl), SO₂N(C₁-C₄-alkyl)₂, COOH, CO—NH₂, CO—NH(C₁-C₄-alkyl), CO—N(C₁-C₄-alkyl)₂, CO—NH(C₅-C₁₂-heteroaryl), NH—CO(C₁-C₄-alkyl), CH₂—NH—CO(C₁-C₄-alkyl), NH—CO(C₅-C₁₂-heteroaryl), CH₂—NH—CO(C₅-C₁₂-heteroaryl), styryl, or by —S(O)ᵣ—CH₃ where r is 0-2, or two adjacent positions may be substituted by —O—CH₂—O— or —O—C(CH₃)₂—O—,
   a monocyclic C₅-C₇-heteroaryl which may optionally be mono- or 10 polysubstituted, identically or differently, by C₁-C₄-alkyl,
   provided that R² cyano or R¹ and/or R² are the same or different and are each a C₁-C₄-alkyl radical, where at least one of the radicals is at least monosubstituted or
   provided that R⁵ is an —SO(O)ₚ—C₁-C₄-alkyl where p is 0-2, a C₁-C₆-acyl, —O—CO—NH(C₁-C₄-alkyl), —O—CO—N(C₁-C₄-alkyl)₂, C₆-C₁₂-aryloxy, C₅-C₁₆-heteroaryloxy, hydroxyl, cyano or N—(C₁-C₄-alkyl)₂,
   a monocyclic C₅-C₇-heteroaryl which may be at least mono- or polysubstituted, identically or differently, by halogen, CF₃, C₁-C₄-acyl, C₁-C₄-alkoxy, hydroxyl, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂,
   a bi- or tricyclic C₈-C₁₂-heteroaryl which may optionally be mono- or polysubstituted, identically or differently, by halogen, by C₁-C₄-alkyl, C₁-C₄-acyl, C₁-C₄-alkoxy, hydroxyl, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂ or
   a C₃-C₆-cycloalkyl which may optionally be mono- or polysubstituted, identically or differently, by halogen, by C₁-C₄-alkyl, hydroxyl, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R⁵ is a hydrogen, halogen, amino, —SO(O)ₚ—C₁-C₄-alkyl where p is 0-2,
   a C₁-C₄-acyl, NH—CO—NH₂—, NH—CO—C₁-C₄-alkyl-, —O—CO—NH(C₁-C₄-alkyl)-, —O—CO—N(C₁-C₄-alkyl)₂ or C₁-C₄-alkyl group which may optionally be mono- or polysubstituted, identically or differently, by C₁-C₄-acyl, C₁-C₄-alkoxy, hydroxyl, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
   a C₁-C₄-alkoxy which may optionally be mono- or polysubstituted, identically or differently, by hydroxyl, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
   an O—C₆-C₁₂-aryl which may optionally be substituted by hydroxyl, cyano, COOH or CO—NH₂,
   a CH₂O—C₆-C₁₂-aryl which may optionally be substituted by hydroxyl, cyano, COOH or CO—NH₂,
   an O—C₅-C₁₆-heteroaryl which may optionally be substituted by hydroxyl, cyano, COOH or CO—NH₂,
   a hydroxyl, cyano, O—CO—(C₁-C₄-alkyl), CO—NH(C₅-C₁₂-heteroaryl), NH—(C₁-C₄-alkyl), N—(C₁-C₄-alkyl)₂,
   a C₆-C₁₂-aryl which may optionally be mono- or polysubstituted, identically or differently, by halogen, by C₁-C₄-alkyl, C₃-C₆-cycloalkyl, C₁-C₄-acyl, C₁-C₄-alkoxy, C₆-C₁₂-aryl, C₅-C₁₂-heteroaryl, hydroxyl, CH₂—OH, cyano, CH₂—CN, amino, CO₂—(C₁-C₄-alkyl), N—(C₁-C₄-alkyl)₂, NHSO₂CH₃, SO₂NH₂, SO₂NH(C₁-C₄-alkyl), SO₂N(C₁-C₄-alkyl)₂, COOH, CO—NH₂, CO—NH(C₁-C₄-alkyl), CO—N(C₁-C₄-alkyl)₂, CO—NH(C₅-C₁₂-heteroaryl), NH—CO(C₁-C₄-alkyl), CH₂—NH—CO(C₁-C₄-alkyl), NH—CO(C₅-C₁₂-heteroaryl), CH₂—NH—CO(C₅-C₁₂-heteroaryl), styryl, or an —S(O)ᵣ—CH₃ where r is 0-2, or two adjacent positions may be substituted by —O—CH₂—O— or —O—C(CH₃)₂—O—,
   a monocyclic C₅-C₇-heteroaryl which may optionally be mono- or polysubstituted, identically or differently, by C₁-C₄-alkyl, provided that R² is cyano or when R¹ and/or R² are the same or different and are each a C₁-C₄-alkyl radical, where at least one of the radicals is at least monosubstituted,
   a monocyclic C₅-C₇-heteroaryl which may be at least mono- or polysubstituted, identically or differently, by halogen, CF₃, C₁-C₄-acyl, C₁-C₄-alkoxy, hydroxyl, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂,
   a bi- or tricyclic C₈-C₁₂-heteroaryl which may optionally be mono- or polysubstituted, identically or differently, by halogen, by C₁-C₄-alkyl, C₁-C₄-acyl, C₁-C₄-alkoxy, hydroxyl, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂ or
   a C₃-C₆-cycloalkyl which may optionally be mono- or polysubstituted, identically or differently, by halogen, by C₁-C₄-alkyl, hydroxyl, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R⁴and R⁵ are in ortho, meta or meta,para positions with respect to one another and together are defined as —O—CO—S—, —S—CO—O—, —CH₂—CO—O—, O—CO—CH₂—, —CH₂—CO—NH—, —NH—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
Y is a —(CH₂)₂ group,

and the isomers, diastereomers, enantiomers and salts or cyclodextrin clathrates thereof.

Preference is likewise given to those compounds of the general formula (I) where
A is a phenyl, naphthyl or C₅-C₁₂-heteroaryl radical which may optionally be mono- or disubstituted by R⁴ and/or R⁵,
R¹ is a C₁-C₄-alkyl radical,
R² is a hydrogen, fluorine, chlorine, bromine or C₁-C₄-alkyl,
R³ is a hydrogen, methyl, ethyl, trifluoromethyl, cyano, chlorine or bromine,
R⁴ is a hydrogen, halogen, amino, an —SO(O)ₚ—C₁-C₄-alkyl group where p is 0-2,
   a C₁-C₄-acyl, NH—CO—NH₂, —O—CO—NH(C₁-C₄-alkyl), —O—CO—N(C₁-C₄-alkyl)₂ or NH—CO—C₁-C₄-alkyl radical,
   a C₁-C₄-alkyl which may optionally be mono-, di- or trisubstituted, identically or differently, by C₁-C₄-acyl, C₁-C₄-alkoxy, hydroxyl, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
   a C₁-C₄-alkoxy which may optionally be mono-, di- or trisubstituted, identically or differently, by hydroxyl, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
   an O—C₆-C₁₂-aryl which may optionally be mono- or disubstituted by hydroxyl, cyano, COOH or CO—NH₂,
   a CH₂O—C₆-C₁₂-aryl which may optionally be mono- or disubstituted by hydroxyl, cyano, COOH or CO—NH₂,
   an O—C₅-C₁₆-heteroaryl which may optionally be mono- or disubstituted by hydroxyl, cyano, COOH or CO—NH₂,
   a hydroxyl, cyano, O—CO—(C₁-C₄-alkyl), CO—NH(C₅-C₁₂-heteroaryl), NH—(C₁-C₄-alkyl), N—(C₁-C₄-alkyl)₂,
   a C₆-C₁₂-aryl which may optionally be mono-, di- or trisubstituted, identically or differently, by halogen, by C₁-C₄-alkyl, C₃-C₆-cycloalkyl, C₁-C₄-acyl, C₁-C₄-alkoxy, C₆-C₁₂-aryl, C₅-C₁₂-heteroaryl, hydroxyl, CH₂—OH, cyano, CH₂—CN, amino, CO₂—(C₁-C₄-alkyl), N—(C₁-C₄-alkyl)₂, NHSO₂CH₃, SO₂NH₂, SO₂NH(C₁-C₄-alkyl), SO₂N(C₁-C₄-alkyl)₂, COOH, CO—NH₂, CO—NH(C₁-C₄-alkyl), CO—N(C₁-C₄-alkyl)₂, CO—NH(C₅-C₁₂-heteroaryl), NH—CO(C₁-C₄-alkyl), CH₂—NH—CO(C₁-C₄-alkyl), NH—CO(C₅-C₁₂-heteroaryl), CH₂—NH—CO(C₅-C₁₂-heteroaryl), styryl, or by —S(O)ᵣ—CH₃ where r is 0-2, or two adjacent positions may be substituted by —O—CH₂—O— or —O—C(CH₃)₂—O—,
   a monocyclic C₅-C₇-heteroaryl which may optionally be mono-, di- or trisubstituted, identically or differently, by C₁-C₄-alkyl,
      provided that R² is cyano or when R¹ and/or R² are the same or different and are each a C₁-C₄-alkyl radical, where at least one of the radicals is at least monosubstituted or
      provided that R⁵ is an —SO(O)ₚ—C₁-C₄-alkyl where p is 0-2, is a C₁-C₆-acyl, —O—CO—NH(C₁-C₄-alkyl), —O—CO—N(C₁-C₄-alkyl)₂, C₆-C₁₂-aryloxy, C₅-C₁₆-heteroaryloxy, hydroxyl, cyano or N—(C₁-C₄-alkyl)₂,
   a monocyclic C₅-C₇-heteroaryl which may be at least mono-, di- or trisubstituted, identically or differently, by halogen, CF₃, C₁-C₄-acyl, C₁-C₄-alkoxy, hydroxyl, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂,
   a bi- or tricyclic C₈-C₁₂-heteroaryl which may optionally be mono-, di- or trisubstituted, identically or differently, by halogen, by C₁-C₄-alkyl, C₁-C₄-acyl, C₁-C₄-alkoxy, hydroxyl, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂ or
   a C₃-C₆-cycloalkyl which may optionally be mono-, di- or trisubstituted, identically or differently, by halogen, by C₁-C₄-alkyl, hydroxyl, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R⁵ is a hydrogen, halogen, amino, —SO(O)ₚ—C₁-C₄-alkyl where p is 0-2,
   a C₁-C₄-acyl, NH—CO—NH₂, NH—CO—C₁-C₄-alkyl, —O—CO—NH(C₁-C₄-alkyl), —O—CO—N(C₁-C₄-alkyl)₂ or C₁-C₄-alkyl group which may optionally be mono-, di- or trisubstituted, identically or differently, by C₁-C₄-acyl, C₁-C₄-alkoxy, hydroxyl, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
   a C₁-C₄-alkoxy which may optionally be mono-, di- or trisubstituted, identically or differently, by hydroxyl, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
   an O—C₆-C₁₂-aryl which may optionally be substituted by hydroxyl, cyano, COOH or CO—NH₂,
   a CH₂O—C₆-C₁₂-aryl which may optionally be substituted by hydroxyl, cyano, COOH or CO—NH₂,
   an O—C₅-C₁₆-heteroaryl which may optionally be substituted by hydroxyl, cyano, COOH or CO—NH₂,
   a hydroxyl, cyano, O—CO—(C₁-C₄-alkyl), CO—NH(C₅-C₁₂-heteroaryl), NH—(C₁-C₄-alkyl), N—(C₁-C₄-alkyl)₂,
   a C₆-C₁₂-aryl which may optionally be mono-, di- or trisubstituted, identically or differently, by halogen, by C₁-C₄-alkyl, C₃-C₆-cycloalkyl, C₁-C₄-acyl, C₁-C₄-alkoxy, C₆-C₁₂-aryl, C₅-C₁₂-heteroaryl, hydroxyl, CH₂—OH, cyano, CH₂—CN, amino, CO₂—(C₁-C₄-alkyl), N—(C₁-C₄-alkyl)₂, NHSO₂CH₃, SO₂NH₂, SO₂NH(C₁-C₄-alkyl), SO₂N(C₁-C₄-alkyl)₂, COOH, CO—NH₂, CO—NH(C₁-C₄-alkyl), CO—N(C₁-C₄-alkyl)₂, CO—NH(C₅-C₁₂-heteroaryl), NH—CO(C₁-C₄-alkyl), CH₂—NH—CO(C₁-C₄-alkyl), NH—CO(C₅-C₁₂-heteroaryl), CH₂—NH—CO(C₅-C₁₂-heteroaryl), styryl, or by —S(O)ᵣ—CH₃ where r is 0-2, or two adjacent positions may be substituted by —O—CH₂—O— or —O—C(CH₃)₂—O—,
   a monocyclic C₅-C₇-heteroaryl which may optionally be mono-, di- or trisubstituted, identically or differently, by C₁-C₄-alkyl,
      provided that R² is cyano or when R¹ and/or R² are the same or different is a C₁-C₄-alkyl radical, where at least one of the radicals is at least monosubstituted,
   a monocyclic C₅-C₇-heteroaryl which may be at least mono-, di- or trisubstituted, identically or differently, by halogen, CF₃, C₁-C₄-acyl, C₁-C₄-alkoxy, hydroxyl, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂,
   a bi- or tricyclic C₈-C₁₂-heteroaryl which may optionally be mono-, di- or trisubstituted, identically or differently, by halogen, by C₁-C₄-alkyl, C₁-C₄-acyl, C₁-C₄-alkoxy, hydroxyl, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂ or
   a C₃-C₆-cycloalkyl which may optionally be mono-, di- or trisubstituted, identically or differently, by halogen, by C₁-C₄-alkyl, hydroxyl, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R⁴ and R⁵ are in ortho, meta or meta,para positions with respect to one another and together are —O—CO—S—, —S—CO—O—, CH₂—CO—O—, O—CO—CH₂—, —CH₂—CO—NH—, —NH—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
Y is a —(CH₂)₂ group,

and the isomers, diastereomers, enantiomers and salts or cyclodextrin clathrates thereof.

Preference is likewise given to those compounds of the general formula (I) where
A is a phenyl, naphthyl or C₅-C₁₂-heteroaryl radical which may optionally be mono- or disubstituted by R⁴ and/or R⁵,
R¹ is a C₁-C₄-alkyl radical,
R² is a hydrogen, fluorine, chlorine, bromine, methyl, ethyl or trifluoromethyl,
R³ is a hydrogen, methyl, ethyl, trifluoromethyl, cyano, chlorine or bromine,
   R⁴ is a hydrogen, halogen, amino,
   a C₁-C₄-acyl or NH—CO—C₁-C₄-alkyl radical,
   a C₁-C₄-alkyl which may optionally be mono- or disubstituted, identically or differently, by C₁-C₄-acyl, C₁-C₄-alkoxy, hydroxyl, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
   a C₁-C₄-alkoxy which may optionally be monosubstituted by hydroxyl, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
   an O-phenyl which may optionally be mono- or disubstituted by hydroxyl, cyano, COOH or CO—NH₂,
   a CH₂O-phenyl which may optionally be mono- or disubstituted by hydroxyl, cyano, COOH or CO—NH₂,
   an O—C₅-C₇-heteroaryl which may optionally be mono- or disubstituted by hydroxyl, cyano, COOH or CO—NH₂,
   a hydroxyl, cyano, O—CO—(C₁-C₄-alkyl), CO—NH(C₅-C₁₂-heteroaryl), NH—(C₁-C₄-alkyl), N—(C₁-C₄-alkyl)₂,
   a phenyl which may optionally be mono- or disubstituted, identically or differently, by halogen, by C₁-C₄-alkyl, C₁-C₄-acyl, C₁-C₄-alkoxy, hydroxyl, CH₂—OH, cyano, CH₂—CN, amino, CO₂—(C₁-C₄-alkyl), N—(C₁-C₄-alkyl)₂, NHSO₂CH₃, SO₂NH₂, SO₂NH(C₁-C₄-alkyl), SO₂N(C₁-C₄-alkyl)₂, COOH, CO—NH₂, CO—NH(C₁-C₄-alkyl), CO—N(C₁-C₄-alkyl)₂, NH—CO(C₁-C₄-alkyl), CH₂—NH—CO(C₁-C₄-alkyl), or two adjacent positions may be substituted by —O—CH₂—O— or —O—C(CH₃)₂—O—,
R⁵ is a hydrogen, halogen, amino,
   a C₁-C₄-acyl or NH—CO—C₁-C₄-alkyl radical,
   a C₁-C₄-alkyl which may optionally be mono- or disubstituted, identically or differently, by C₁-C₄-acyl, C₁-C₄-alkoxy, hydroxyl, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
   a C₁-C₄-alkoxy which may optionally be monosubstituted by hydroxyl, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
   an O-phenyl which may optionally be mono- or disubstituted by hydroxyl, cyano, COOH or CO—NH₂,
   a CH₂O-phenyl which may optionally be mono- or disubstituted by hydroxyl, cyano, COOH or CO—NH₂,
   an O—C₅-C₇-heteroaryl which may optionally be mono- or disubstituted by hydroxyl, cyano, COOH or CO—NH₂,
   a hydroxyl, cyano, O—CO—(C₁-C₄-alkyl), CO—NH(C₅-C₁₂-heteroaryl), NH—(C₁-C₄-alkyl), N—(C₁-C₄-alkyl)₂,
   a phenyl which may optionally be mono- or disubstituted, identically or differently, by halogen, by C₁-C₄-alkyl, C₁-C₄-acyl, C₁-C₄-alkoxy, hydroxyl, CH₂—OH, cyano, CH₂—CN, amino, CO₂—(C₁-C₄-alkyl), N—(C₁-C₄-alkyl)₂, NHSO₂CH₃, SO₂NH₂, SO₂NH(C₁-C₄-alkyl), SO₂N(C₁-C₄-alkyl)₂, COOH, CO—NH₂, CO—NH(C₁-C₄-alkyl), CO—N(C₁-C₄-alkyl)₂, NH—CO(C₁-C₄-alkyl), CH₂—NH—CO(C₁-C₄-alkyl), or two adjacent positions may be substituted by —O—CH₂—O— or —O—C(CH₃)₂—O—,
R⁴ and R⁵ are in ortho, meta or meta,para positions with respect to one another and together are defined as —CH₂—CO—NH—, —NH—CO—CH₂—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
Y is a —(CH₂)₂ group,

and the isomers, diastereomers, enantiomers and salts or cyclodextrin clathrates thereof.

The following compounds according to the present invention are very particularly preferred:
1. 3,4-dimethoxy-N-[2-(2-methyl-1H-indol-3-yl)ethyl]benzamide
2. N-[2-(7-chloro-2-methyl-1H-indol-3-yl)ethyl]-5-fluoro-1H-indole-2-carboxamide
3. N-[2-(7-chloro-4-fluoro-2-methyl-1H-indol-3-yl)ethyl]-5-fluoro-1H-indole-2-carboxamide
4. 3-N-methyl-4′-N′-[2-(7-chloro-4-fluoro-2-methyl-1H-indol-3-yl)ethyl]- biphenyl-3,4′-dicarboxamide
5. N-[2-(7-chloro-2-methyl-1H-indol-3-yl)ethyl]-6-(3-methylcarbamoyl-phenyl)nicotinamide
6. N-[2-(7-chloro-2-methyl-1H-indol-3-yl)ethyl]-3′-hydroxybiphenyl-4-carboxamide
7. N-[2-(7-chloro-4-fluoro-2-methyl-1H-indol-3-yl)ethyl]-3,4-dimethoxy-benzamide
8. 3-N-methyl-4′-N′-[2-(2-methyl-1H-indol-3-yl)ethyl]biphenyl-3,4′-dicarboxamide
9. N-[2-(2-methyl-1H-indol-3-yl)ethyl]-5-fluoro-1H-indole-2-carboxamide
10. N-[2-(2-methyl-1H-indol-3-yl)ethyl]-5-chloro-1H-indole-2-carboxamide
11. N-[2-(2,4-dimethyl-1H-indol-3-yl)ethyl]-3,4-dimethoxybenzamide
12. N-[2-(7-chloro-2,4-dimethyl-1H-indol-3-yl)ethyl]-3,4-dimethoxybenzamide
13. N-[2-(7-bromo-2,4-di methyl-1H-indol-3-yl)ethyl]-3,4-dimethoxybenzamide
14. N-[2-(7-bromo-2,4-dimethyl-1H-indol-3-yl)ethyl]-3H-benzotriazole-5-carboxamide
15. N-[2-(7-bromo-2,4-dimethyl-1H-indol-3-yl)ethyl]-1H-indole-2-carboxamide
16. N-[2-(7-bromo-2,4-di methyl-1H-indol-3-yl)ethyl]quinoxaline-6-carboxamide
17. N-[2-(7-chloro-2,4-dimethyl-1H-indol-3-yl)ethyl]-3H-benzotriazole-5-carboxamide
18. N-[2-(7-chloro-2,4-dimethyl-1H-indol-3-yl)ethyl]quinoxaline-6-carboxamide
19. N-[2-(7-chloro-2,4-dimethyl-1H-indol-3-yl)ethyl]-1H-indole-2-carboxamide
20. N-[2-(7-bromo-2-methyl-1H-indol-3-yl)ethyl]-3,4-dimethoxybenzamide
21. 3-N-methyl-4′-N′-[2-(7-bromo-2-methyl-1H-indol-3-yl)ethyl]biphenyl-3,4′-dicarboxamide
22. N-[2-(7-bromo-2-methyl-1H-indol-3-yl)ethyl]-5-fluoro-1H-indole-2-carboxamide
23. N-[2-(7-chloro-2-trifluoromethyl-1H-indol-3-yl)ethyl]-3,4-dimethoxy-benzamide
24. N-[2-(7-chloro-2-trifluoromethyl-1H-indol-3-yl)ethyl]-5-fluoro-1H-indole-2-carboxamide
25. N-[2-(7-chloro-2-trifluoromethyl-1H-indol-3-yl)ethyl]-5-chloro-1H-indole-2-carboxamide
26. 3-N-methyl-4′-N′-[2-(7-chloro-2-trifluoromethyl-1H-indol-3-yl)ethyl]biphenyl-3,4′-dicarboxamide
27. N-[2-(7-chloro-2-trifluoromethyl-1H-indol-3-yl)ethyl]-5-trifluoromethoxy-1H-indole-2-carboxamide
28. N-[2-(7-chloro-2-trifluoromethyl-1H-indol-3-yl)ethyl]benzofuran-2-carboxamide
29. N-[2-(7-chloro-2-trifluoromethyl-1H-indol-3-yl)ethyl]benzo[b]thiophene-2-carboxamide
30. N-[2-(7-bromo-2-methyl-1H-indol-3-yl)ethyl]-5-chloro-1H-indole-2-carboxamide
31. N-[2-(7-chloro-2-methyl-1H-indol-3-yl)ethyl]-5-chloro-1H-indole-2-carboxamide
32. N-[2-(2-tert-butyl-1H-indol-3-yl)ethyl]-3,4-dimethoxybenzamide
33. N-[2-(2-tert-butyl-1H-indol-3-yl)ethyl]-5-trifluoromethoxy-1H-indole-2-carboxamide
34. N-[2-(2-tert-butyl-1H-indol-3-yl)ethyl]-5-chloro-1H-indole-2-carboxamide
35. N-[2-(2-tert-butyl-1H-indol-3-yl)ethyl]-5-trifluoromethyl-1H-indole-2-carboxamide
36. N-[2-(2-tert-butyl-1H-indol-3-yl)ethyl]-5-bromo-1H-indole-2-carboxamide
37. N-[2-(2-tert-butyl-1H-indol-3-yl)ethyl]-5-fluoro-1H-indole-2-carboxamide
38. 2-bromo-N-[2-(2,7-di methyl-1H-indol-3-yl)ethyl]-4,5-dimethoxybenzamide
39. N-[2-(2,7-dimethyl-1H-indol-3-yl)ethyl]-1H-indole-6-carboxamide
40. N-[2-(2,7-dimethyl-1H-indol-3-yl)ethyl]quinoline-5-carboxamide
41. N-[2-(2,7-dimethyl-1H-indol-3-yl)ethyl]-5-phenyl-1H-pyrazole-4-carboxamide
42. N-[2-(2,7-dimethyl-1H-indol-3-yl)ethyl]pyridazine-4-carboxamide
43. N-[2-(2,7-dimethyl-1H-indol-3-yl)ethyl]-5-phenyl-2H-pyrazole-3-carboxamide
44. N-[2-(2,7-dimethyl-1H-indol-3-yl)ethyl]pyrimidine-5-carboxamide
45. N-[2-(2,7-dimethyl-1H-indol-3-yl)ethyl]pyrazolo[1,5-a]pyridine-2-carboxamide
46. N-[2-(2,7-dimethyl-1H-indol-3-yl)ethyl]benzo[b]thiophene-5-carboxamide
47. N-[2-(2,7-dimethyl-1H-indol-3-yl)ethyl]quinoxaline-2-carboxamide
48. N-[2-(2,7-dimethyl-1H-indol-3-yl)ethyl]-3-fluoro-2-methoxybenzamide
49. 3-chloro-N-[2-(2,7-dimethyl-1H-indol-3-yl)ethyl]-2-methylbenzamide
50. 3-chloro-N-[2-(2,7-dimethyl-1H-indol-3-yl)ethyl]-2-fluorobenzamide
51. N-[2-(2,7-dimethyl-1H-indol-3-yl)ethyl]-4-phenoxybenzamide
52. N-[2-(2,7-dimethyl-1H-indol-3-yl)ethyl]thiazole-4-carboxamide
53. 2-chloro-N-[2-(2,7-dimethyl-1H-indol-3-yl)ethyl]-3-methylbenzamide
54. N-[2-(2,7-dimethyl-1H-indol-3-yl)ethyl]quinoline-4-carboxamide
55. N-[2-(2,7-dimethyl-1H-indol-3-yl)ethyl]-3-hydroxybenzamide
56. N-[2-(2,7-dimethyl-1H-indol-3-yl)ethyl]-4-hydroxy-2-phenyl-2H-pyrazole-3-carboxamide
57. N-[2-(2,7-dimethyl-1H-indol-3-yl)ethyl]benzo[b]thiophene-3-carboxamide
58. N-[2-(2,7-dimethyl-1H-indol-3-yl)ethyl]-1H-indole-2-carboxamide
59. N-[2-(2,7-dimethyl-1H-indol-3-yl)ethyl]quinoline-2-carboxamide
60. N-[2-(2,7-dimethyl-1H-indol-3-yl)ethyl]furan-2-carboxamide
61. 2-chloro-N-[2-(2,7-dimethyl-1H-indol-3-yl)ethyl]nicotinamide
62. N-[2-(2,7-dimethyl-1H-indol-3-yl)ethyl]quinoline-3-carboxamide
63. 5-bromo-N-[2-(2,7-di methyl-1H-indol-3-yl)ethyl]nicotinamide
64. N-[2-(2,7-dimethyl-1H-indol-3-yl)ethyl]-1H-indole-3-carboxamide
65. 4-benzyloxy-N-[2-(2,7-dimethyl-1H-indol-3-yl)ethyl]benzamide
66. N-[2-(2,7-dimethyl-1H-indol-3-yl)ethyl]-3-bromothiophene-2-carboxamide
67. N-[2-(2,7-dimethyl-1H-indol-3-yl)ethyl]-2-methylfuran-3-carboxamide
68. N-[2-(2,7-dimethyl-1H-indol-3-yl)ethyl]-4-oxo-4,5,6,7-tetrahydro-benzo[b]thiophene-2-carboxamide
69. 2-benzo[1,2,5]thiadiazol-4-yl-N-[2-(2,7-dimethyl-1H-indol-3-yl)ethyl]-acetamide
70. N-[2-(2,7-dimethyl-1H-indol-3-yl)ethyl]-1H-imidazole-4-carboxamide
71. N-[2-(2,7-dimethyl-1H-indol-3-yl)ethyl]-4′-bromobiphenyl-2-carboxamide
72. N-[2-(2,7-dimethyl-1H-indol-3-yl)ethyl]-2-fluoro-6-iodobenzamide
73. 3′-[2-(2,7-dimethyl-1H-indol-3-yl)ethylcarbamoyl]biphenyl-2-carboxylic acid methyl ester
74. N-[2-(2,7-dimethyl-1H-indol-3-yl)ethyl]-2,3-dihydrobenzofuran-7-carboxamide
75. N-[2-(2,7-dimethyl-1H-indol-3-yl)ethyl]-3-fluoro-2-methylbenzamide
76. N-[2-(2,7-dimethyl-1H-indol-3-yl)ethyl]-2,5-dimethylbenzamide
77. N-[2-(2,7-dimethyl-1H-indol-3-yl)ethyl]-5-acetylthiophene-2-carboxamide
78. N-[2-(2,7-dimethyl-1H-indol-3-yl)ethyl]-2-fluoro-3-methoxybenzamide
79. N-[2-(2,7-dimethyl-1H-indol-3-yl)ethyl]quinoline-8-carboxamide
80. N-[2-(2,7-dimethyl-1H-indol-3-yl)ethyl]-4-methylsulfanylbenzamide
81. N-[2-(2,7-dimethyl-1H-indol-3-yl)ethyl]-2-phenyl-2H-pyrazole-3-carboxamide
82. N-[2-(2,7-dimethyl-1H-indol-3-yl)ethyl]-6-phenylpyrimidine-4-carboxamide
83. N-[2-(2,7-dimethyl-1H-indol-3-yl)ethyl]-1-methyl-1H-indole-3-carboxamide
84. N-[2-(2,7-dimethyl-1H-indol-3-yl)ethyl]-2-phenoxymethylbenzamide
85. N-[2-(2,7-dimethyl-1H-indol-3-yl)ethyl]-2,5-dimethyl-2H-pyrazole-3-carboxamide
86. N-[2-(2,7-dimethyl-1H-indol-3-yl)ethyl]-2,3-dihydrobenzofuran-5-carboxamide
87. N-[2-(2,7-dimethyl-1H-indol-3-yl)ethyl]-5-methoxy-1H-indole-2-carboxamide
88. N-[2-(2-methyl-7-trifluoromethyl-1H-indol-3-yl)ethyl]-5-methoxy-1H-indole-2-carboxamide
89. N-[2-(2-methyl-7-trifluoromethyl-1H-indol-3-yl)ethyl]-1H-indole-2-carboxamide
90. N-[2-(2-methyl-7-trifluoromethyl-1H-indol-3-yl)ethyl]-5-fluoro-1H-indole-2-carboxamide
91. N-[2-(2-methyl-7-trifluoromethyl-1H-indol-3-yl)ethyl]-5-methyl-1H-indole-2-carboxamide
92. N-[2-(2-methyl-7-trifluoromethyl-1H-indol-3-yl)ethyl]-6-methoxy-1H-indole-2-carboxamide
93. N-[2-(2-methyl-7-trifluoromethyl-1H-indol-3-yl)ethyl]-4-methyl-1H-indole-2-carboxamide
94. N-[2-(2-methyl-7-trifluoromethyl-1H-indol-3-yl)ethyl]-4-methoxy-1H-indole-2-carboxamide
95. N-[2-(2-methyl-7-trifluoromethyl-1H-indol-3-yl)ethyl]-4-fluoro-1H-indole-2-carboxamide
96. N-[2-(2-methyl-7-trifluoromethyl-1H-indol-3-yl)ethyl]-6-fluoro-1H-indole-2-carboxamide
97. N-[2-(2-methyl-7-trifluoromethyl-1H-indol-3-yl)ethyl]-6-methyl-1H-indole-2-carboxamide
98. N-[2-(2-methyl-7-trifluoromethyl-1H-indol-3-yl)ethyl]-7-methyl-1H-indole-2-carboxamide
99. N-[2-(7-cyano-2,4-dimethyl-1H-indol-3-yl)ethyl]-3,4-dimethoxybenzamide
100. N-[2-(7-chloro-2-methyl-1H-indol-3-yl)ethyl]-3,4-dimethoxybenzamide
101. 4-N-methyl-4′-N′-[2-(7-chloro-2-methyl-1H-indol-3-yl)ethyl]biphenyl-4,4′-dicarboxamide
102. 3-N-methyl-4′-N′-[2-(7-chloro-2-methyl-1H-indol-3-yl)ethyl]biphenyl-3,4′-dicarboxamide
103. N-[2-(7-bromo-2,4-di methyl-1H-indol-3-yl)ethyl]-3,4-dimethoxybenzamide
104. N-[2-(4,7-dichloro-2-methyl-1H-indol-3-yl)ethyl]-3,4-dimethoxybenzamide
105. 3-N-methyl-4′-N′-[2-(4,7-dichloro-2-methyl-1H-indol-3-yl)ethyl]biphenyl-3,4′-dicarboxamide

The present invention provides for the use of the inventive compounds for the production of medicaments which comprise at least one of the compounds of the formula I.

The present invention likewise provides medicaments which comprise the inventive compounds with suitable formulation and carrier substances.

Compared with known prostaglandin E₂ ligands, the novel EP₂ agonists and antagonists are distinguished by greater selectivity and stability.

The present invention provides medicaments for the treatment and prophylaxis of disorders which include fertility disorders, infectious disorders, cancer, viral infections, cardiovascular disorders, elevated intraocular pressure, glaucoma, skeletal system disorders, angiogenetic disorders, uterine contraction impairments, pain, neuroinflammatory disorders, immunomodulatory infections and nephrological disorders.

Fertility disorders mean the disorders which lead to no ovulation taking place, no nidation of a fertilized oocyte occurring and no decidualization taking place, infectious disorders mean disorders caused by unicellular parasites, cancer means solid tumours and leukaemia, viral infections mean for example cytomegalus infections, hepatitis, hepatitis B and C and HIV disorders, immunomodulatory infections mean for example avian influenza, cardiovascular disorders mean ischaemic reperfusion disorder, stenoses, arterioscleroses and restenoses, angiogenetic disorders mean for example endometriosis and fibrosis, elevated intraocular pressure means glaucoma, uterine contraction impairments mean for example menstrual complaints, skeletal system disorders mean osteoporosis, neuroinflammatory disorders mean multiple sclerosis, Alzheimer's disease, Parkinson's disease, Crohn's disease, ulcerative colitis, pain and nephrological disorders mean polycystic kidney disorder, glomerulonephritis.

The present invention likewise provides medicaments for the treatment and prophylaxis of the disorders detailed above, which comprise at least one lo compound of the general formula I, and medicaments with suitable formulation and carrier substances.

For the use of the inventive compounds as medicaments, they are converted to the form of a pharmaceutical product which, as well as the active ingredient, comprises inert organic or inorganic pharmaceutical carrier materials which are suitable for enteral or parenteral administration, such as, for example, water, gelatin, gum arabic, lactose, starch, magnesium stearate, talc, vegetable oils, polyalkylene glycols etc. The pharmaceutical products may be in solid form, for example as tablets, coated tablets, suppositories, capsules, in semisolid form, for example as ointments, creams, gels, suppositories, emulsions or in liquid form, for example as solutions, suspensions or emulsions.

They comprise where appropriate excipients which are intended to act for example as fillers, binders, disintegrants, lubricants, solvents, solubilizers, masking flavours, colorant, emulsifiers. Examples of types of excipients for the purpose of the invention are saccharides (mono-, di-, tri-, oligo-, and/or polysaccharides), fats, waxes, oils, hydrocarbons, anionic, nonionic, cationic natural, synthetic or semisynthetic surfactants. They additionally comprise where appropriate excipients such as preservatives, stabilizers, wetting agents or emulsifiers; salts to modify the osmotic pressure or buffers.

The present invention likewise provides these pharmaceutical products.

It is appropriate to produce aerosol solutions for inhalation.

Suitable for oral use are in particular tablets, coated tablets or capsules with talc and/or hydrocarbon carriers or binders, such as, for example, lactose, corn starch or potato starch. Use can also take place in liquid form, such as, for example, as solution to which, where appropriate, a sweetener is added. Clathrates are likewise also suitable for oral use of such compounds, examples of clathrates which may be mentioned being those with alpha-, beta-, gamma-cyclodextrin or else beta-hydroxypropylcyclodextrin.

Sterile, injectable, aqueous or oily solutions are used for parenteral administration. Particularly suitable are injection solutions or suspensions, especially aqueous solutions of active compounds in polyethoxylated castor oil.

Examples suitable and customary for vaginal administration are pessaries, tampons or an intrauterine system.

Appropriately prepared crystal suspensions can be used for intraarticular injection.

It is possible to use for intramuscular injection aqueous and oily injection solutions or suspensions and appropriate depot preparations.

For rectal administration, the novel compounds can be used in the form of suppositories, capsules, solutions (e.g. in the form of enemas) and ointments both for systemic and for local therapy.

The novel compounds can be used in the form of aerosols and inhalations for pulmonary administration.

For local use on the eyes, external auditory canal, middle ear, nasal cavity and paranasal sinuses, the novel compounds can be used as drops, ointments and tinctures in appropriate pharmaceutical preparations.

Formulations possible for topical application are gels, ointments, fatty ointments, creams, pastes, dusting powders, milk and tinctures. The dosage of the compounds of the general formula I should in these preparations be 0.01%-20% in order to achieve an adequate pharmacological effect.

The dosage of the active ingredients may vary depending on the route of administration, age and weight of the patient, nature and severity of the disorder to be treated and similar factors. Treatment can take place by single dosages or by a large number of dosages over a prolonged period. The daily dose is 0.5-1000 mg, preferably 50-200 mg, it being possible to give the dose as a single dose to be administered once or divided into 2 or more daily doses.

Carrier systems which can be used are also surface-active excipients such as salts of bile acids or animal or vegetable phospholipids, but also mixtures thereof, and liposomes or constituents thereof.

The present invention likewise provides the formulations and dosage forms described above.

Administration of the compounds of the invention can take place by any conventional method, including oral and parenteral, e.g. by subcutaneous or intramuscular injections. The present invention likewise provides enteral, parenteral, vaginal and oral administrations.

The compounds of the invention of the general formula I bind to the EP₂ receptor and have agonistic or antagonistic effect. It is possible to determine whether an agonistic or an antagonistic effect is present by an agonism test (see Example 1.2.1. of the Biological Examples) or by an antagonism test (see Example 1.2.2. of the Biological Examples).

Antagonists mean molecules which bind to their corresponding receptors and which inhibit the initiation of the signal transduction pathway(s) coupled to the receptor by the naturally occurring ligand(s). The antagonists normally compete with the naturally occurring ligand of the receptor for binding to the receptor. However, other modifications of the receptor are also possible by molecules which prevent the signal transduction pathways coupled to the receptor being activated by the naturally occurring ligand(s) (e.g. non-competitive, steric modifications of the receptor).

Receptor antagonists typically bind selectively to their particular receptor and not to other receptors. They normally have a higher binding affinity than the natural ligand. Although antagonists which have a higher affinity for the receptor lo than the natural ligand are preferred, it is likewise possible to employ antagonists having a lower affinity.

The antagonists preferably bind reversibly to their corresponding receptors.

The EP₂ receptor antagonist has a preferential affinity for the EP₂ receptor compared with any other EP receptor. The antagonism is measured in the presence of the natural agonist (PGE₂).

Agonists mean molecules which bind to their corresponding receptors and normally compete with the naturally occurring ligand of the receptor for binding to the receptor, and which stimulate the initiation of the signal transduction pathway coupled to the receptor. Agonists may also assist the binding of the natural ligand.

Receptor agonists typically bind selectively to their particular receptor and not to other receptors. They normally have a higher binding affinity than the natural ligand. Although agonists which have a higher affinity for the receptor than the natural ligand are preferred, it is likewise possible to employ agonists having a lower affinity.

The agonists preferably bind reversibly to their corresponding receptors.

The EP₂ receptor agonist has a preferred affinity for the EP₂ receptor compared with any other EP receptor.

Agonists are tested via the initiation of the signal transduction and/or physiological effect mediated by the corresponding receptor.

The compounds or low molecular weight substances which bind to a receptor are referred to as ligands. Their binding is normally reversible. Binding of a ligand to the corresponding receptor activates or inactivates the signal transduction pathway coupled to the receptor. The ligand mediates its intracellular effect in this manner. Ligands mean agonists and antagonists of a receptor.

The substance of Example 7 shows no inhibition in the cellular agonism test but a good activity (IC₅₀=0.047×10 E-6 M) in the antagonism test.

The present invention likewise provides for the use of the substances of the invention as EP₂ receptor antagonists for the treatment of disorders which are caused by disturbances in the signal transduction chain in which the EP₂ receptor is involved, such as, for example, pain and fertility disorders, and which are likewise suitable for controlling fertility.

The oocyte is surrounded in the preovulatory antral follicle by cumulus cells which form a dense ring of cells around the oocyte. After the lutenizing hormone peak (LH peak), a series of processes is activated and leads to a large morphological change in this ring of cells composed of cumulus cells. In this case, the cumulus cells form an extracellular matrix which leads to so-called cumulus expansion (Vanderhyden et al. Dev Biol. August 1990;140(2):307-317). This cumulus expansion is an important constituent of the ovulatory process and of the subsequent possibility of fertilization.

Prostaglandins, and here prostaglandin E₂, whose synthesis is induced by the LH peak, are of crucial importance in cumulus expansion. Prostanoid EP₂ knockout mice (Hizaki et al. Proc Natl Acad Sci USA. Aug. 31, 1999;96(18):10501-6.) show a distinctly reduced cumulus expansion and severe subfertility, demonstrating the importance of the prostanoid EP₂ receptor for this process.

The substances of the invention have inhibitory effects in cumulus expansion tests.

The present invention provides for the use of the substances of the invention for controlling fertility.

While the EP₂ receptor antagonist AH 6809 suppresses the expansion of the cumulus by only approx. 30% only at a concentration of 100-200 μM, it is possible to achieve approx. 60% suppression of cumulus expansion in the presence of the substance of example 8, even at a concentration lower by 20-40-fold (5 μM). In these tests, the test substances compete with the natural EP₂ receptor agonist PGE₂.

The present invention provides for the use of the substances of the invention for inhibiting cumulus expansion and thus ovulation and fertilization for contraception.

Prostaglandins play an important part in angiogenesis (Sales, Jabbour, 2003, Reproduction 126, 559-567; Kuwano et al., 2004, FASEB J. 18, 300-310; Kamiyama et al., 2006, Oncogene 25, 7019-7028; Chang et al. 2005, Prostaglandins & other Lipid Mediators 76, 48-58).

Endometriosis is a chronic disorder caused by impairments of blood vessels. About 10% of women regularly suffer from heavy bleeding during menstruation, caused by changes in the blood vessels of the endometrium. In addition, structural differences in the blood vessels have been observed, such as, for example, incomplete formation of the smooth muscle cell layer (Abberton et al., 1999, Hum. Reprod. 14, 1072-1079). Since the blood loss during menstruation is partly controlled by constriction of the blood vessels, it is obvious that the defects in the smooth muscles make a substantial contribution to the bleeding.

The present invention provides for the use of the substances of the general formula I for treating endometriosis.

Prostaglandins play an important part in uterine contraction, and excessively strong contractions are responsible for menstrual complaints (Sales, Jabbour, 2003, Reproduction 126, 559-567), Jabbour, Molecular and Cellular Endocrinology 252 (2006) 191-200). Recent studies indicate that the EP₂ receptor is involved in heavy bleeding during menstruation (Smith et al. Human Reproduction 2007; 22(5): 1450-1456).

The present invention provides for the use of the substances of the general formula I for the treatment of menstrual complaints.

Increasing research results also demonstrate the importance of EP receptors, and especially of the EP₂ receptor, in a large number of types of cancer (e.g. breast cancer, colon carcinoma, lung cancer, prostate cancer, leukaemia, skin cancer, oesophageal cancer), suggesting future possibilities of employing modulators (antagonists or agonists) of the EP₂ receptor for the therapy and prevention (prophylactic and/or adjuvant) of cancer (Fulton et al. Cancer Res 2006; 66(20): 9794-7; Pan et al. 2008; The Journal of Biological Chemistry 283(17): 11155-11163; Subbaramaiah et al. 2008; The Journal of Biological Chemistry 283(6): 3433-3444; Castellone et al. Science VOL 310 2005, 1504-1510; Chang et al. Cancer Res 2005; 65(11): 4496-9); Hull et al. Mol Cancer Ther 2004;3(8):1031-9; Richards et al. J Clin Endocrinol Metab 88: 2810-2816, 2003; Sinha et al. 2007, Cancer Res; 67(9):4507-13; Wang et al. 2004, Seminars in Oncology, Vol 31, No 1, Suppl 3: pp 64-73; Yu et al. 2008; JPET Published on Jun. 26, 2008 as DOI:10.1124/jpet.108.141275).

The present invention provides for the use of the substances of the general formula I for the treatment and prevention of cancers.

Prostaglandins also play an important part in processes counteracting osteoporosis. The present invention therefore provides for the use of the substances of the invention for the treatment of osteoporosis.

Reinold et al. (J. Clin. Invest. 115, 673-679 (2005)) describes PGE₂ receptors of the EP₂ subtype as the key signalling elements in inflammatory hyperalgesia. Mice no longer having this receptor (EP₂^{-/-}) do not experience spinal inflammatory pain. There is evidence that an inflammatory, increased pain sensitivity can be treated by targeted modulation of EP₂ receptors.

The present invention provides for the use of the substances of the invention for the treatment of inflammatory hyperalgesia.

Prostaglandins and especially the EP₂ receptor are also associated with β-amyloid formation in Alzheimer's disease (Hoshino et al. 2007 J Biol Chem.; 282(45) :32676-3288).

The present invention provides for the use of the inventive substances for the prevention and treatment of Alzheimer's disease.

The EP₂ receptor-mediated effects of PGE₂ are likewise associated with Parkinson's disease (Jin et al. 2007; Journal of Neuroinflammation 1186/1742-2094-4-2).

The present invention provides for the use of the inventive substances for the prevention and treatment of Parkinson's disease.

Owing to its immunomodulatory effects, the EP₂ receptor plays a part in inflammatory bowel disorders (Crohn's disease, ulcerative colitis) (Sheibanie et al. 2007; The Journal of Immunology, 178: 8138-8147.)

The present invention provides for the use of the inventive substances for the prevention and treatment of inflammatory bowel disorders, for example Crohn's disease, ulcerative colitis.

Recent studies show that the EP₂ receptor is involved in the development of polycystic kidneys. EP₂ receptor antagonists may be an approach to the prevention and treatment of this disorder (Elberg et al. 2007, Am J Physiol Renal Physiol 293: F1622-F1632.)

The present invention provides for the use of the inventive substances for the prevention and treatment of polycystic kidney disorders.

The EP₂ receptor is likewise associated with atherosclerotic development processes (Lie et al. 2006, Circ Res.;98:642-650).

The present invention provides for the use of the inventive substances for the prevention and treatment of atherosclerosis.

Serezani et al. (Am Respir Cell Mol Biol Vol 37. pp 562-570, 2007) state that the activation of the EP₂ receptor by PGE₂ macrophages of the respiratory tract impairs its ability to destroy bacteria. Bacterial infections lead to increased production of prostaglandins, including PGE₂, which weakens the endogenous defense against bacteria through this mechanism. As shown in this publication, an inactivation of the EP₂ receptor (and of the EP₄ receptor) can re-establish this ability to fight bacteria. Further relevant publications which explain these connections are: Sadikot et al. Eur. J. Immunol. 2007. 37: 1001-1009 and Aronoff et al. The Journal of Immunology, 2004,173: 559-565.

The present invention provides for the use of the inventive substances for the treatment of infection disorders of the lung.

The natural ligand (agonist) of the EP₂ receptor is PGE₂, whose synthesis is mediated via cyclooxygenase (COX) enzymes (COX-1, COX-2). These enzymes are involved in the pathological states mentioned, and the indications and the development thereof, usually through enhanced expression and activity. Therefore, in all possible uses mentioned, a combination of a COX inhibitor (COX-2 and/or COX-1) is possible, with the aim of
a) achieving a higher and more effective pharmacological efficacy than with a substance class and
b) enabling a lower dosage of one of the two or both substance classes, which leads to a reduction in possible side effects and better tolerance.

The present invention therefore also provides medicaments comprising a compound of the general formula (I) in combination with a COX inhibitor for treatment of disorders. Examples of COX inhibitors include the nonselective COX inhibitors such as aspirin, naproxen, indomethacin, ibuprofen, or the selective COX inhibitors meloxicam, celecoxib (4-[5-(4-methylphenyl)-3-(trifluoromethyl)-1H-pyrazol-1-yl]benzenesulphonamide), parecoxib (N-[4-(5-methyl-3-phenyl-4-isoxazolyl)phenyl]sulphonylpropionamide), rofecoxib (4-(4-mesylphenyl)-3-phenylfuran-2(5H)-one), valdecoxib (4-[5-methyl-3-phenyl-4-isoxazoyl)benzenesulphonamide), NS-398 (N-methyl-2-cyclohexanoxy-4-nitrobenzenesulphonamide), lumiracoxib [2-(2′-chloro-6′-fluorophenyl)amino-5-methylbenzeneacetic acid, ceracoxib and etoricoxib.

The invention also relates to a process for preparing the compounds of the general formula I, which is characterized in that a compound of the formula II

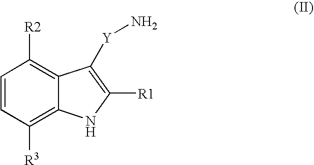

in which R¹, R², R³ and Y are each as defined above was reacted with a carboxylic acid derivative of the general formula III

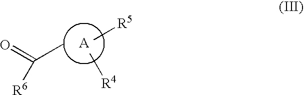

in which A, R⁴ and R⁵ are each as defined above and R⁶ may be a hydroxyl group, a chlorine or bromine atom or a C₁-C₆-alkyl radical, preferably hydrogen, chlorine, the methyl or ethyl radical, by methods known to those skilled in the art, and any protecting groups required were then detached.

When R⁶ is a hydroxyl group, the reaction can be effected first by activating the acid function; this is done, for example, by first converting the carboxylic acid of the formula III to the mixed anhydride in the presence of a tertiary amine, for example triethylamine, with isobutyl chloroformate. The mixed anhydride was reacted with the alkali metal salt of the corresponding amine in an inert solvent or solvent mixture, for example tetrahydrofuran, dimethoxyethane, dimethylformamide, hexamethylphosphoramide, at temperatures between −30° C. and +60° C., preferably at 0° C. to 30° C.

A further possibility was to activate the carboxylic acid by means of reagents, for example HOBt (N-hydroxybenzotriazole) or HATU (o-(7-azabenzotriazol-1-yl)-N,N,N′,N′-tetramethyluronium hexafluorophosphate). The acid was reacted, for example, with HATU in an inert solvent, for example DMF, in the presence of the corresponding amine of the general formula III and a tertiary amine, for example ethyldiisopropylamine, at temperatures between −50° C. and +60° C., preferably at 0° C. to 30° C., or alternatively between 80° C. and 140° C. in a microwave.

A further possibility is to first convert the acid function in the compounds of the general function II to the corresponding acid chloride by means of, for example, thionyl chloride, phosphorus oxychloride, phosphorus pentachloride or else oxalyl chloride, and then to perform the conversion to the compounds of the general formula I, for example, in pyridine or an inert solvent, for example DMF, in the presence of the corresponding amine of the general formula III or IV and a tertiary amine, for example ethyldiisopropylamine at temperatures between −50° C. and +60° C., preferably at 0° C. to 30° C.

When R⁶ is C₁-C₆-alkyl, it is, for example, also possible to perform a direct amidolysis of the ester with the corresponding amine, possibly with the aid of aluminium trialkyl reagents, preferably aluminium trimethyl.

When R⁶ is a chlorine or bromine atom, it is possible, for example, to perform the reaction, for example, in pyridine or an inert solvent, for example DMF, in the presence of the corresponding amine of the general formula II and a tertiary amine, for example ethyldiisopropylamine, at temperatures between −50° C. and +60° C., preferably at 0° C. to 30° C.

In some cases, the compounds of the general formula (I) where R² or R³═CN can also be prepared proceeding from the corresponding halides, preference being given to bromine or chlorine, by a Cu- or Pd-catalysed (e.g. Pd(AOc)₂) cyanide introduction with Zn(CN)₂ or else K₃[Fe(CN)₆] in an inert solvent such as dimethylacetamide, dimethylformamide or N-methylpyrrolidone at temperatures between 60° C. and the boiling point of the particular solvent.

In some cases, the compounds of the general formula (I) where R⁴ or R⁵=aryl or heteroaryl, which may optionally be substituted by the above-specified radicals, can be prepared proceeding from a corresponding halide, preference being given to bromine or chlorine, by a Pd-catalysed (e.g. Pd(AOc)₂, Pd(PPh₃)₄, Pd₂(dba)₃, PdCl₂(dppf)) reaction in the presence of a base, for example sodium carbonate, caesium carbonate, potassium phosphate or ethyldiisopropylamine, with a corresponding aryl- or heteroarylboronic acid or boronic acid derivative in a solvent, for example toluene, dioxane, dimethylacetamide, dimethylformamide or N-methyl-pyrrolidone, at temperatures between 60° C. and the boiling point of the particular solvent.

The compounds of the general formula II which serve as starting materials are either known or can be prepared, for example, in a manner known per se by reacting the known hydrazines IV, if appropriate prepared from the corresponding known anilines by nitrosation followed by a reduction,

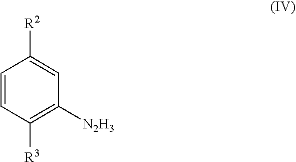

in which R² and R³ are each as defined above,

a) with a ketone of the general formula V in which R¹ and Y are each as defined above and n=2 and 3, in a Fischer indole cyclization,

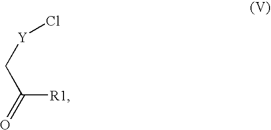

or

b) with an enol ether of the general formula VI in which R¹ and Y are each as defined above and n=2 and 3, in a Fischer indole cyclization (Org. Lett. 2004, 79ff),

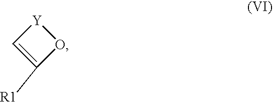

and the alcohol obtained subsequently is converted to the amino function by the methods known to those skilled in the art, by conversion to a leaving group such as tosylate, mesylate, trifluoromesylate, chloride, bromide or iodide, and subsequent reaction with, for example, sodium azide, followed by a hydrolysis by means of triphenylphosphine/water in tetrahydrofuran,

or

c) with a keto ester of the general formula VII, in the case of Y where n=1

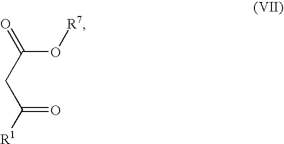

in which R¹ is as defined above and R⁷ is a C₁-C₆-alkyl radical, in a Fischer indole cyclization and then reducing the resulting ester to the corresponding alcohol by methods known to those skilled in the art, for example diisobutylaluminium hydride in an inert solvent at temperatures between −50 and 25° C., preferably between −30 and 0° C., and in turn converting said alcohol to the amino function by conversion to a leaving group such as tosylate, mesylate, trifluoromesylate, chloride, bromide or iodide and subsequent reaction with, for example, sodium azide, followed by a hydrolysis by means of triphenylphosphine/water in tetrahydrofuran.

A further alternative for preparation of the compounds of the general formula II would first be the reaction of known indoles VIII in which R¹ to R³ are each as defined above, or indoles VIII building on indole syntheses known to those skilled in the art (Chem. Rev. 2006, 2875 or J. Chem. Soc., Perkin Trans 1, 2000, 1045),

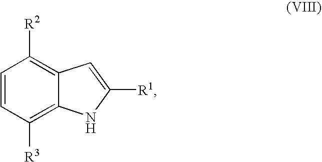

in which R¹ to R³ are each as defined above with formaldehyde/dimethylamine in the presence of a base, for example potassium carbonate, in an inert solvent, for example dioxane, at temperatures between 0° C. and the boiling point of the particular solvent, preferably between 60° C. and 80° C., to give the compounds of the general formula IX

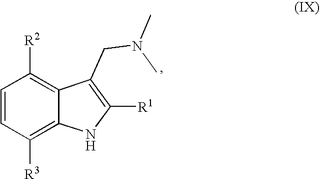

in which R¹ to R³ are each as defined above, are reacted. The compounds of the general formula IX are then converted to the nitrile extended by one carbon atom by reaction of sodium cyanide or potassium cyanide in a solvent mixture such as, preferably, DMF/water under reflux, and said nitrile then gives rise to the compounds of the general formula VII through a reduction with lithium aluminium hydride in an inert solvent, for example diethyl ether or tetrahydrofuran, under reflux or alternatively by means of sodium borohydride/cobalt diacetate in ethanol or methanol, preferably at temperatures between 10° C. and 40° C.

Should protecting groups be necessary for reactions, they can be introduced at preliminary stages or for the step required by methods known to those skilled in the art (Protective groups in organic synthesis, T. W. Greene and P. G. M. Wuts, John Wiley & Sons, 1999), and if appropriate, subsequently or at a later stage in the synthesis, also be detached again.

Preparation of the Inventive Compounds

The examples which follow explain the preparation of the inventive compounds of the general formula (I), without restricting the scope of the compounds claimed to these examples.

The inventive compounds of the general formula (I) can be prepared as described below.

### EXAMPLE 1

### N-[2-(7-chloro-2-methyl-1H-indol-3-yl)ethyl]-3,4-dimethoxy-benzamide

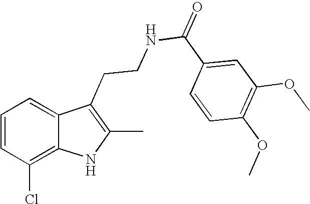

To a solution of 100 mg of 2-(7-chloro-2-methyl-1H-indol-3-yl)ethylamine hydrochloride in 6 ml of tetrahydrofuran (or alternatively dimethylformamide) was added 0.14 ml of triethylamine, and the mixture was stirred at 25° C. for 10 minutes. Subsequently, at this temperature, 123 mg of 3,4-dimethoxybenzoyl chloride were added, and the mixture was stirred at 25° C. for a further 15 hours. Subsequently, the reaction solution was added to ice-water and extracted twice with ethyl acetate. The combined organic phases were washed twice with water, dried over sodium sulphate, filtered and then concentrated under reduced pressure. The residue thus obtained is purified by moderate pressure chromatography on silica gel with hexane/0-100% ethyl acetate. Yield: 103 mg of the title compound.

NMR (300 MHz, DMSO-d6): δ=2.30 (3H), 2.84 (2H), 3.35 (2H), 3.75 (3H), 3.76 (3H), 6.90 (1H), 6.97 (1H), 7.01 (1H), 7.36-7.45 (3H), 8.41 (1H), 11.00 (1H).

### EXAMPLE 2

### 4-N-methyl-4′-N′-[2-(7-chloro-2-methyl-1H-indol-3-yl)ethyl]-biphenyl-4,4′-dicarboxamide

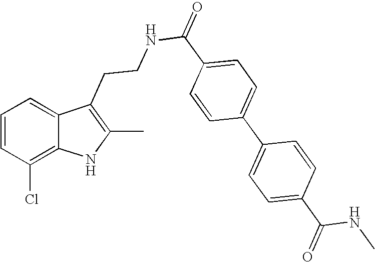

To a solution of 156 mg of 4-N-methylbiphenyl-4,4′-dicarboxamide in 1.7 ml of dimethyl sulphoxide (alternatively also dimethylformamide) were added 256 mg of N-[(dimethylamino)-1H-1,2,3-triazolo[4,5-b]pyridin-1-ylmethylene]-N-methyl-methanaminium hexafluorophosphate N-oxide (HATU) and 100 mg of 2-(7-chloro-2-methyl-1H-indol-3-yl)ethylamine hydrochloride. At 0° C., 0.12 ml of ethyldiisopropylamine were then added dropwise and the mixture was stirred at 25° C. for 20 hours. The mixture was purified by HPLC (column: XBridge C18 5μ 100×30 mm, eluent: 99% of a mixture of water with 0.1% formic acid/1% acetonitrile up to 1% of a mixture of water with 0.1% formic acid/99% acetonitrile, detection by MS ESI (+)). Yield: 38 mg of the title compound.

NMR (300 MHz, DMSO-d6): δ=2.35 (3H), 2.82 (3H), 2.92 (2H), 3.44 (2H), 6.96 (1H), 7.06 (1H), 7.47 (1H), 7.84 (4H), 7.95 (4H), 8.53 (1H), 8.67 (1H), 11.05 (1H).

### EXAMPLE 3

### 3-N-methyl-4′-N′-[2-(7-chloro-2-methyl-1H-indol-3-yl)ethyl]-biphenyl-3,4′-dicarboxamide

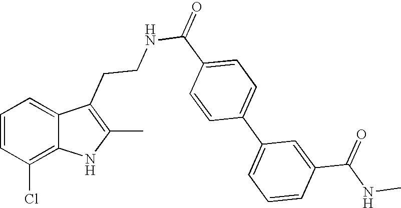

In analogy to Example 2, 100 mg of 2-(7-chloro-2-methyl-1H-indol-3-yl)-ethylamine hydrochloride and 183 mg of 3-N-methylbiphenyl-3,4′-dicarboxamide were used to obtain 48 mg of the title compound.

NMR (300 MHz, DMSO-d6): δ=2.36 (3H), 2.83 (3H), 2.92 (2H), 3.45 (2H), 6.96 (1H), 7.06 (1H), 7.48 (1H), 7.58 (1H), 7.81-7.99 (6H), 8.18 (1H), 8.59 (1H), 8.67 (1H), 11.05 (1H).

### EXAMPLE 4

### N-[2-(7-bromo-2,4-dimethyl-1H-indol-3-yl)ethyl]-3,4-dimethoxy-benzamide

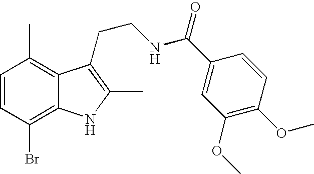

In analogy to Example 1, 100 mg of 2-(7-bromo-2,4-dimethyl-1H-indol-3-yl)-ethylamine hydrochloride and 90 mg of 3,4-dimethoxybenzoyl chloride were used to obtain 37 mg of the title compound.

NMR (300 MHz, DMSO-d6): δ=2.30 (3H), 2.61 (3H), 2.94 (2H), 3.31 (2H), 3.76 (3H), 3.77 (3H), 6.60 (1H), 6.98 (1H), 7.01 (1H), 7.40 (1H), 7.44 (1H), 8.49 (1H), 10.81 (1H).

The starting material for the above title compound was prepared as follows:

4a) 2-(7-bromo-2,4-dimethyl-1H-indol-3-yl)ethylamine

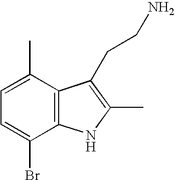

5.8 g of 2-bromo-5-methylphenylhydrazine hydrochloride were dissolved in 78 ml of a mixture of ethanol and water in a ratio of 14:1 at 120° C. Subsequently, at boiling, 2.8 ml of 5-chloro-2-pentanone dissolved in 2 ml of ethanol were added, and the mixture was stirred at this temperature for 16 hours. After cooling, the mixture was concentrated under reduced pressure and admixed with 100 ml of water and sodium hydroxide solution (pH approx. 10). The mixture was then extracted three times with 1:1 ether/ethyl acetate, and the combined organic phases were washed with water and dried over sodium sulphate. After filtration, the mixture was concentrated under reduced pressure and the residue thus obtained was purified on silica gel with methylene chloride/0-20% methanol/0.5% triethylamine. Yield: 2.45 g of the title compound.

NMR (300 MHz, DMSO-d6): δ=2.30 (3H), 2.51 (3H), 2.62 (2H), 2.75 (2H), 6.57 (1H), 6.98 (1H), 10.75 (1H).

4b) 2-bromo-5-methylphenylhydrazine hydrochloride

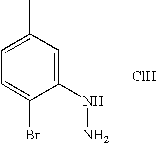

To a solution of 5.0 g of 2-bromo-5-methylaniline in 17 ml of hydrochloric acid (37%) was added dropwise, at 0° C. within 30 minutes, a solution of 1.9 g of sodium nitrite in 9.6 ml of water. Subsequently, at 0° C., a solution of 16.6 g of tin chloride in 15 ml of hydrochloric acid (37%) was added dropwise, and the mixture was stirred at this temperature for a further 1.5 hours. After adding 60 ml of sodium hydroxide solution (50%) and 30 ml of ice-water (pH>10), the mixture was extracted three times with 500 ml each time of ether. The combined organic phases were washed with semisaturated sodium chloride solution and dried over sodium sulphate. The filtrate was acidified with 4.0M hydrochloric acid in 1,4-dioxane solution and the resulting precipitate was then filtered off and dried. Yield: 5.85 g of the title compound.

NMR (300 MHz, DMSO-d6): δ=2.22 (3H), 6.70 (1H), 6.93 (1H), 7.39 (1H), 7.78 (1H), 10.29 (2H).

### EXAMPLE 5

### N-[2-(4,7-dichloro-2-methyl-1H-indol-3-yl)ethyl]-3,4-dimethoxybenzamide

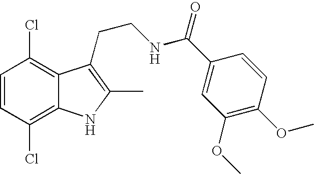

In analogy to Example 1, 50 mg of 2-(4,7-dichloro-2-methyl-1H-indol-3-yl)ethylamine hydrochloride and 36 mg of 3,4-dimethoxybenzoyl chloride were used to obtain 36 mg of the title compound.

NMR (300 MHz, DMSO-d6): δ=2.27 (3H), 3.04 (2H), 3.41 (2H), 3.75 (3H), 3.76 (3H), 6.94 (1H), 6.96 (1H), 7.01 (1H), 7.38 (1H), 7.41 (1H), 8.39 (1H), 11.37 (1H).

Starting materials required for compounds in the table below:

A) 2-(7-chloro-4-fluoro-2-methyl-1H-indol-3-yl)ethylamine

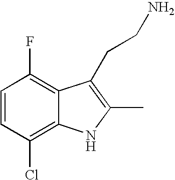

In analogy to Example 4a), 6 g of 2-chloro-4-fluorophenylhydrazine hydrochloride were used to obtain 1.8 g of 2-(7-chloro-4-fluoro-2-methyl-1H-indol-3-yl)ethylamine.

NMR (300 MHz, DMSO-d6): δ=2.32 (3H), 2.91 (4H), 6.72 (1H), 6.99 (1H), 11.45 (1H).

B) 2-(7-chloro-2,4-dimethyl-1H-indol-3-yl)ethylamine

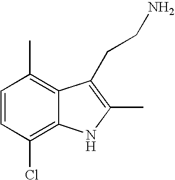

B1) In analogy to Example 4b), 5.0 g of 2-chloro-5-methylphenylamine were used to obtain 6.5 g of (2-chloro-5-methylphenyl)hydrazine hydrochloride.

B2) In analogy to Example 4a), 6.5 g of the hydrazine prepared above were used to obtain 2.75 g of 2-(7-chloro-2,4-dimethyl-1H-indol-3-yl)-ethylamine.

NMR (300 MHz, DMSO-d6): δ=2.30 (3H), 2.64 (2H), 2.77 (2H), 6.61 (1H), 6.84 (1H), 10.90 (1H).

C) 2-(7-bromo-2-methyl-1H-indol-3-yl)ethylamine

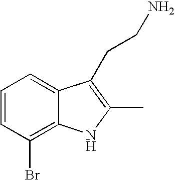

In analogy to Example 4a), 6.5 g of 2-bromophenylhydrazine hydrochloride were used to obtain 7.2 g of 2-(7-bromo-2-methyl-1H-indol-3-yl)ethylamine.

NMR (300 MHz, DMSO-d6): δ=2.34 (3H), 2.98 (4H), 6.87 (1H), 7.17 (1H), 7.46 (1H), 11.0 1 (1H).

D) 2-(7-chloro-2-trifluoromethyl-1H-indol-3-yl)ethylamine

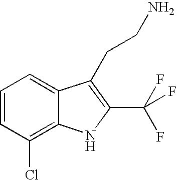

D1) (2-amino-3-chlorophenyl)methanol

To a solution of 10 g of lithium aluminium hydride in 1 l of THF were added cautiously, at 10° C. in portions, 30 g of 2-amino-3-chlorobenzoic acid, and then the mixture was heated under reflux for 2 hours. After cooling, 10 ml of water were added dropwise, followed by a solution of 3.3 g of sodium hydroxide in 10 ml of water, and the mixture was heated under reflux for 30 minutes. After cooling and filtration, the mixture was concentrated under reduced pressure and the residue thus obtained was purified by column chromatography on silica gel with 19:1 chloroform/methanol. Yield: 19.1 g of (2-amino-3-chlorophenyl)methanol.

NMR (300 MHz, DMSO-d6): δ=4.53 (2H), 5.08 (2H), 5.20 (1H), 6.58 (1H), 7.13 (1H), 7.17 (1H).

D2) N-(2-chloro-6-hydroxymethylphenyl)-2,2,2-trifluoroacetamide

To a solution of 18.8 g of the amine prepared above in 400 ml of methylene chloride were added dropwise, at 0° C., 50.1 g of trifluoroacetic anhydride, and the mixture was then stirred at 24° C. for 16 hours. The organic phase was then washed with 15% potassium carbonate solution, dried over sodium sulphate, filtered and then dried under reduced pressure. The residue was extracted with hot hexane. After concentrating under reduced pressure, without further purification, 10.5 g of N-(2-chloro-6-hydroxymethylphenyl)-2,2,2-trifluoroacetamide were obtained.

NMR (300 MHz, DMSO-d6): δ=5.46 (2H), 7.54 (1H), 7.58 (1H), 7.75 (1H), 11.40 (1H).

D3) 7-chloro-2-trifluoromethyl-1H-indole

A mixture of 10.5 g of the amide prepared above and 15.6 g of triphenylphosphine hydrobromide (PPh₃×HBr) in 300 ml of acetonitrile was heated under reflux for 17 hours. After cooling, the mixture was concentrated under reduced pressure and the residue was washed with 200 ml of ether. After drying under air, 23.9 g of phosphonium salt were obtained, which were dissolved in 600 ml of DMF and then heated under reflux for 20 hours. After cooling, the mixture was concentrated under reduced pressure and the residue thus obtained was purified by column chromatography on silica gel with hexane. Yield: 4.2 g of 7-chloro-2-trifluoromethyl-1H-indole.

NMR (300 MHz, DMSO-d6): δ=7.53 (1H), 7.62 (1H), 7.81 (1H), 7.62 (1H), 12.58 (1H).

D4) (7-chloro-2-trifluoromethyl-1H-indol-3-ylmethyl)dimethylamine

To a solution of 1.98 g of potassium carbonate in 40 ml of acetic acid were added, at −10° C., 2.33 g of dimethylamine hydrochloride in 40 ml of dioxane, followed by 1.74 ml of 40% formaldehyde solution and 4.2 g of the indole prepared above in 40 ml of dioxane. Subsequently, this mixture was stirred at 25° C. for 2 hours and then heated to 80° C. for a further 5 hours. After cooling, the reaction mixture was then concentrated under reduced pressure and added to 15% potassium carbonate solution. After extracting three times with 100 ml of ethyl acetate each time, the combined organic phases were dried over sodium sulphate. After filtration, the mixture was concentrated under reduced pressure and the residue thus obtained was purified by means of column chromatography on silica gel with 19:1 hexane/ethyl acetate. Yield: 4.46 g of the title compound as a solid.

NMR (300 MHz, DMSO-d6): δ=2.15 (6H), 3.61 (2H), 7.11 (1H), 7.36 (1H), 7.76 (1H), 12.32 (1H).

D5) (7-chloro-2-trifluoromethyl-1H-indol-3-yl)acetonitrile

To a solution of 4.4 g of the amine prepared beforehand in 50 ml of DMF was added a solution of 10.35 g of potassium cyanide in 50 ml of water and this mixture was heated under reflux for 2 hours. After cooling, the mixture was concentrated under reduced pressure and the residue thus obtained was purified by column chromatography on silica gel with 9:1 hexane/ethyl acetate. Yield: 2.37 g of the title compound as a solid.

NMR (300 MHz, DMSO-d6): δ=4.28 (2H), 7.22 (1H), 7.44 (1H), 7.86 (1H), 12.76 (1H).

D6) 2-(7-chloro-2-trifluoromethyl-1H-indol-3-yl)ethylamine

To a solution of 0.5 g of the nitrile prepared above in a mixture of 7 ml of THF and 2 ml of water, a solution of 250 mg of cobalt diacetate tetrahydrate in 5 ml of water was added at 25° C. with stirring, followed by 720 mg of sodium borohydride, and then the mixture was stirred at this temperature for 30 minutes. The mixture was then concentrated under reduced pressure and the residue thus obtained was purified by column chromatography on silica gel with chloroform/methanol/aq. ammonia in a ratio of 100:10:1. Yield: 200 mg of the title compound as a solid.

NMR (300 MHz, DMSO-d6): δ=2.70 (2H), 2.88 (2H), 5.14 (2H), 7.08 (1H), 7.31 (1H), 7.66 (1H).

E) 2-(2-tert-butyl-1H-indol-3-yl)ethylamine

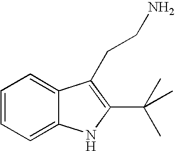

E1) (2-tert-butyl-1H-indol-3-ylmethyl)dimethylamine

In analogy to the compound prepared in D4), 1.0 g of 2-tert-butyl-1H-indole was used to obtain 1.21 g of (2-tert-butyl-1H-indol-3-ylmethyl)dimethylamine as a white solid.

NMR (300 MHz, DMSO-d6): δ=1.46 (9H), 3.52 (2H), 6.95 (2H), 7.27 (1H), 7.50 (1H), 10.56 (1H).

E2) (2-tert-butyl-1H-indol-3-yl)acetonitrile

In analogy to the compound prepared in D5), 6.21 g of the amine (E1) prepared above were used to obtain 4.7 g of (2-tert-butyl-1H-indol-3-yl)-acetonitrile as a white solid.

NMR (300 MHz, DMSO-d6): δ=1.47 (9H), 4.10 (2H), 7.03 (2H), 7.34 (1H), 7.53 (1H), 10.78 (1H).

E3) 2-(2-tert-butyl-1H-indol-3-yl)ethylamine

To a mixture of 1.0 g of lithium aluminium hydride in 400 ml of ether were added cautiously 1.1 g of the nitrile (E2) prepared above, and the mixture was heated under reflux for 48 hours. After cooling, 1 ml of water followed by a solution of 0.3 g of sodium hydroxide in 1 ml of water were added cautiously in succession, and this mixture was once again heated under reflux. After cooling, the solids were filtered off and the filtrate was concentrated under reduced pressure. Yield (without further purification): 0.76 g of 2-(2-tert-butyl-1H-indol-3-yl)ethylamine as a white solid.

NMR (300 MHz, DMSO-d6): δ=1.44 (9H), 2.72 (2H), 2.88 (2H), 6.89 (1H), 6.95 (1H), 7.27 (1H), 7.41 (1H), 10.36 (1H).

In analogy to Example 1 or 2, the following examples are prepared and purified by HPLC:

HPLC Method:

Instrument: analytical 4-channel MUX system with CTC Pal injector, Waters 1525 pumps, Waters 2488 UV detector and Waters ZQ 2000 single quad MS detector.

Column: X-Bridge RP C18 4.6×50 3.5 μm; detection wavelength 214 nm; flow rate 2 ml/min; eluent A: 0.1% TFA in water, B 0.1% TFA in acetonitrile; gradient, based in each case on B: 1% to 99% (5′) to 99% (1′) to 1% (0.25′) to 1% (1.75′)

| | | | Theoretical | Mass | Retention |
|---|---|---|---|---|---|
| | | | mass | found | time |
| Example | Structure | Name | m/z [M]⁺ | m/z [M + H]⁺ | [min.] |
| 6 | 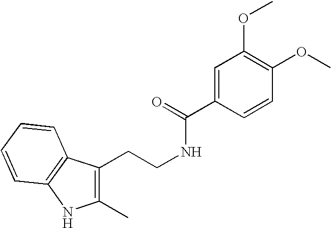 | 3,4-dimethoxy-N-[2- (2-methyl-1H-indol- 3-yl)ethyl]benzamide | 338 | 339 | 1.09 |
| 7 | 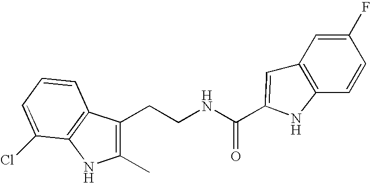 | N-[2-(7-chloro-2- methyl-1H-indol-3- yl)ethyl]-5-fluoro-1H- indole-2- carboxamide | 369 | 370 | 1.25 |
| 8 | 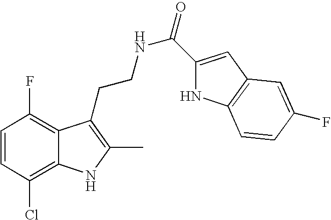 | N-[2-(7-chloro-4- fluoro-2-methyl-1H- indol-3-yl)ethyl]-5- fluoro-1H-indole-2- carboxamide | 387 | 388 | 1.34 |
| 9 | 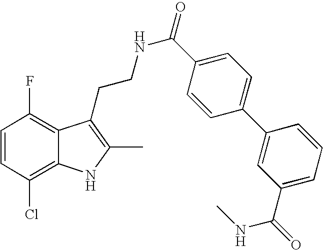 | 3-N-methyl-4′-N′-[2- (7-chloro-4-fluoro-2- methyl-1H-indol-3- yl)ethyl]biphenyl-3,4′- dicarboxamide | 463 | 464 | 1.24 |
| 10 | 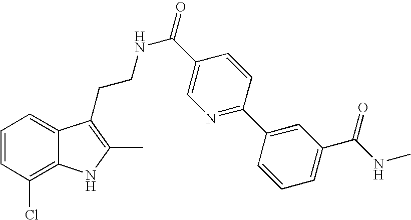 | N-[2-(7-chloro-2- methyl-1H-indol-3- yl)ethyl]-6-(3- methylcarbamoyl- phenyl)nicotinamide | 446 | 447 | 1.15 |
| 11 | 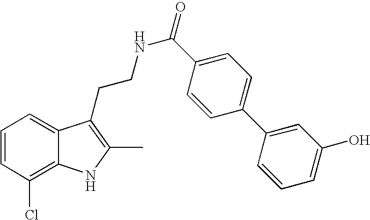 | N-[2-(7-chloro-2- methyl-1H-indol-3- yl)ethyl]-3′- hydroxybiphenyl-4- carboxamide | 404 | 405 | 1.27 |
| 12 | 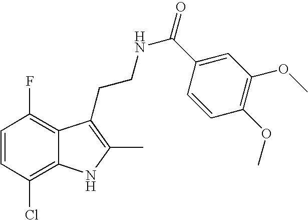 | N-[2-(7-chloro-4- fluoro-2-methyl-1H- fluoro-2-methyl-1H- indol-3-yl)ethyl]-3,4- dimethoxybenzamide | 390 | 391 | 1.25 |
| 13 | 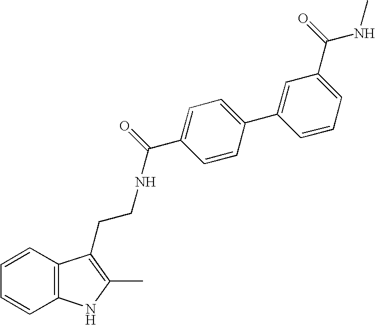 | 3-N-methyl-4′-N′-[2- (2-methyl-1H-indol- 3-yl)ethyl]biphenyl- 3,4′-dicarboxamide | 411 | 412 | 1.13 |
| 14 | 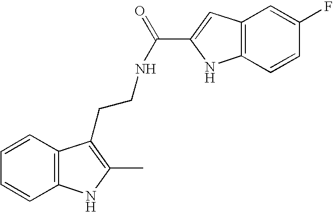 | N-[2-(2-methyl-1H- indol-3-yl)ethyl]-5- fluoro-1H-indole-2- carboxamide | 335 | 336 | 1.24 |
| 15 | 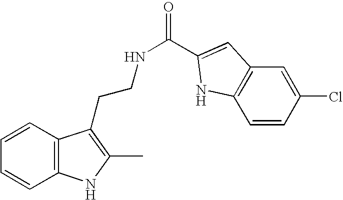 | N-[2-(2-methyl-1H- indol-3-yl)ethyl]-5- chloro-1H-indole-2- carboxamide | 351 | 352 | 1.31 |
| 16 | 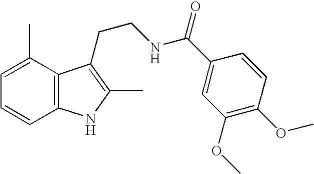 | N-[2-(2,4-dimethyl- 1H-indol-3-yl)ethyl]- 3,4-dimethoxybenz- amide | 352 | 353 | 1.15 |
| 17 | 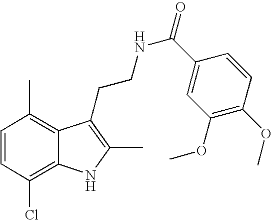 | N-[2-(7-chloro-2,4- dimethyl-1H-indol-3- yl)ethyl]-3,4-di- methoxybenzamide | 386 | 387 | 1.23 |
| 18 | 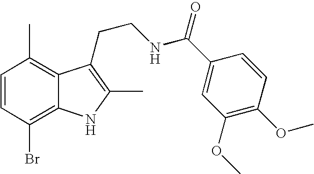 | N-[2-(7-bromo-2,4- dimethyl-1H-indol-3- yl)ethyl]-3,4-di- methoxybenzamide | 432 | 433 | 1.27 |
| 19 | 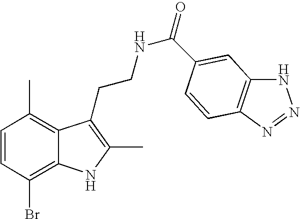 | N-[2-(7-bromo-2,4- dimethyl-1H-indol-3- yl)ethyl]-3H- benzotriazole-5- carboxamide | 413 | 414 | 1.03 |
| 20 | 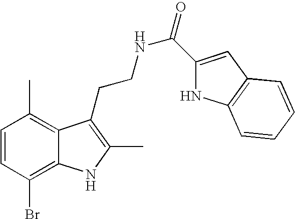 | N-[2-(7-bromo-2,4- dimethyl-1H-indol-3- yl)ethyl]-1H-indole-2- carboxamide | 411 | 412 | 1.3 |
| 21 | 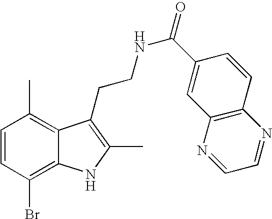 | N-[2-(7-bromo-2,4- dimethyl-1H-indol-3- yl)ethyl]quinoxaline- 6-carboxamide | 424 | 425 | 1.13 |
| 22 | 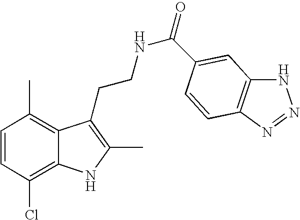 | N-[2-(7-chloro-2,4- dimethyl-1H-indol-3- yl)ethyl]-3H- benzotriazole-5- carboxamide | 367 | 368 | 1.07 |
| 23 | 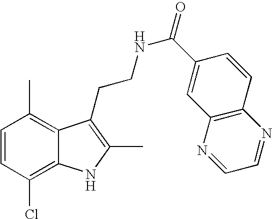 | N-[2-(7-chloro-2,4- dimethyl-1H-indol-3- yl)ethyl]quinoxaline- 6-carboxamide | 378 | 379 | 1.19 |
| 24 | 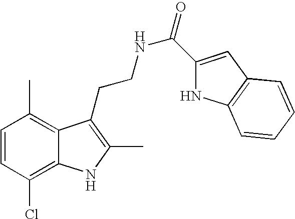 | N-[2-(7-chloro-2,4- dimethyl-1H-indol-3- yl)ethyl]-1H-indole-2- carboxamide | 365 | 366 | 1.36 |
| 25 | 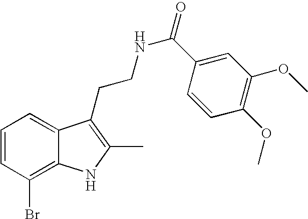 | N-[2-(7-bromo-2- methyl-1H-indol-3- yl)ethyl]-3,4-di- methoxybenzamide | 418 | 419 | 1.24 |
| 26 | 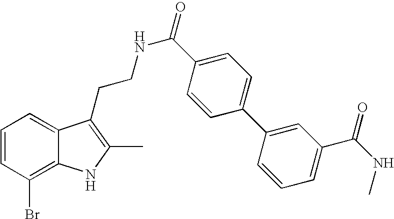 | 3-N-methyl-4′-N′-[2- (7-bromo-2-methyl- 1H-indol-3- yl)ethyl]biphenyl-3,4′- dicarboxamide | 491 | 492 | 1.26 |
| 27 | 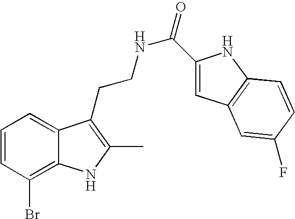 | N-[2-(7-bromo-2- methyl-1H-indol-3- yl)ethyl]-5-fluoro-1H- indole-2- carboxamide | 415 | 416 | 1.36 |
| 28 | 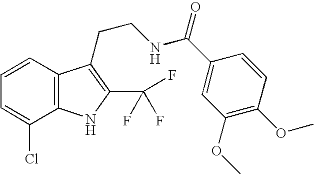 | N-[2-(7-chloro-2- trifluoromethyl-1H- indol-3-yl)ethyl]-3,4- dimethoxybenz- amide | 426 | 427 | 1.24 |
| 29 | 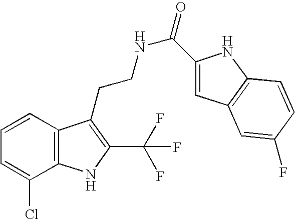 | N-[2-(7-chloro-2- trifluoromethyl-1H- indol-3-yl)ethyl]-5- fluoro-1H-indole-2- carboxamide | 423 | 424 | 1.37 |
| 30 | 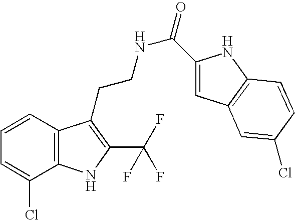 | N-[2-(7-chloro-2- trifluoromethyl-1H- indol-3-yl)ethyl]-5- chloro-1H-indole-2- carboxamide | 439 | 438 [M − H]⁻ | 1.43 |
| 31 | 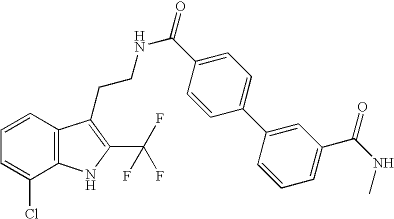 | 3-N-methyl-4′-N′-[2- (7-chloro-2- trifluoromethyl- 1H-indol-3- yl)ethyl]biphenyl-3,4′- dicarboxamide | 499 | 498 | 1.26 |
| 32 | 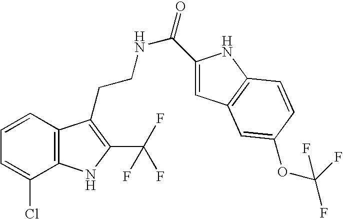 | N-[2-(7-chloro-2- trifluoromethyl-1H- indol-3-yl)ethyl]-5- trifluoromethoxy-1H- indole-2- carboxamide | 489 | 488 [M − H]⁻ | 1.48 |
| 33 | 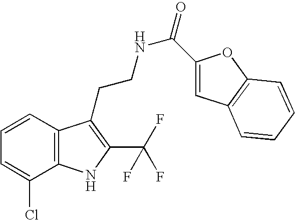 | N-[2-(7-chloro-2- trifluoromethyl-1H- indol-3-yl)ethyl]- benzofuran-2- carboxamide | 406 | 405 [M − H]⁻ | 1.39 |
| 34 | 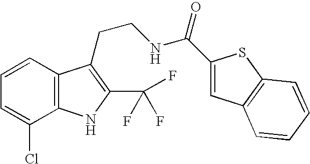 | N-[2-(7-chloro-2- trifluoromethyl-1H- indol-3-yl)ethyl]- benzo[b]thiophene- 2-carboxamide | 422 | 421 [M − H]⁻ | 1.43 |
| 35 | 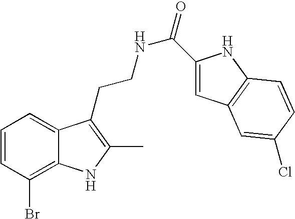 | N-[2-(7-bromo-2- methyl-1H-indol-3- yl)ethyl]-5-chloro-1H- indole-2- carboxamide | 430 | 431 | 1.43 |
| 36 | 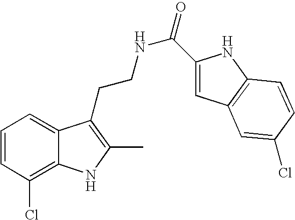 | N-[2-(7-chloro-2- methyl-1H-indol-3- yl)ethyl]-5-chloro-1H- indole-2- carboxamide | 385 | 386 | 1.39 |
| 37 | 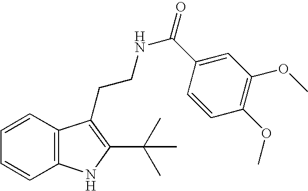 | N-[2-(2-tert-butyl-1H- indol-3-yl)ethyl]-3,4- dimethoxybenz- amide | 380 | 381 | 1 .26 |
| 38 | 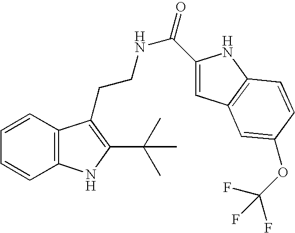 | N-[2-(2-tert-butyl-1H- indol-3-yl)ethyl]-5- trifluoromethoxy-1H- indole-2- carboxamide | 443 | 444 | 1.51 |
| 39 | 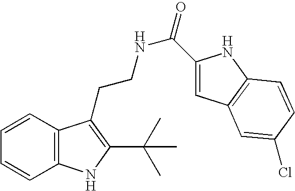 | N-[2-(2-tert-butyl-1H- indol-3-yl)ethyl]-5- chloro-1H-indole-2- carboxamide | 393 | 394 | 1.46 |
| 40 | 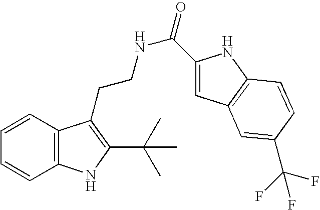 | N-[2-(2-tert-butyl-1H- indol-3-yl)ethyl]-5- trifluoromethyl-1H- indole-2- carboxamide | 427 | 428 | 1.49 |
| 41 | 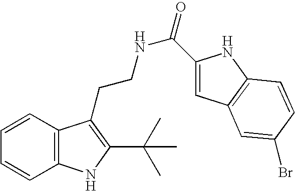 | N-[2-(2-tert-butyl-1H- indol-3-yl)ethyl]-5- bromo-1H-indole-2- carboxamide | 439 | 440 | 1.48 |
| 42 | 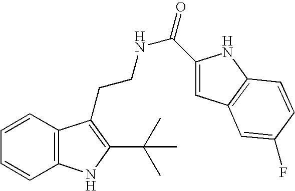 | N-[2-(2-tert-butyl-1H- indol-3-yl)ethyl]-5- fluoro-1H-indole-2- carboxamide | 377 | 378 | 1.39 |
| 43 | 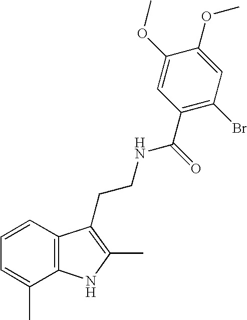 | 2-bromo-N-[2-(2,7- dimethyl-1H-indol-3- yl)ethyl]-4,5-di- methoxybenzamide | 431 | 432 | 9.1 |
| 44 | 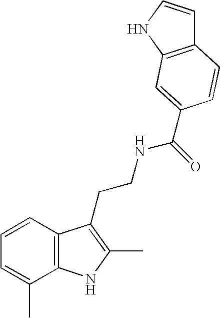 | N-[2-(2,7-dimethyl- 1H-indol-3-yl)ethyl]- 1H-indole-6- carboxamide | 331 | 332 | 8.88 |
| 45 | 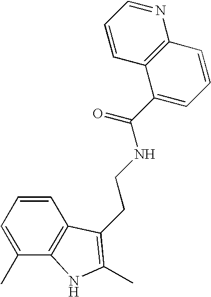 | N-[2-(2,7-dimethyl- 1H-indol-3-yl)ethyl]- quinoline-5- carboxamide | 343 | 344 | 6.57 |
| 46 | 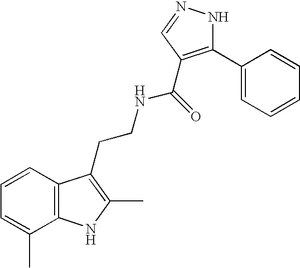 | N-[2-(2,7-dimethyl- 1H-indol-3-yl)ethyl]- 5-phenyl-1H- pyrazole-4- carboxamide | 358 | 359 | 8.26 |
| 47 | 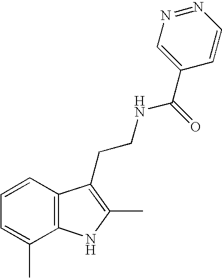 | N-[2-(2,7-dimethyl- 1H-indol-3- yl)ethyl]pyridazine-4- carboxamide | 294 | 295 | 7.22 |
| 48 | 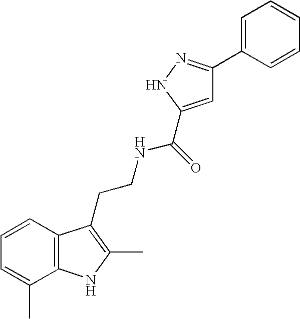 | N-[2-(2,7-dimethyl- 1H-indol-3-yl)ethyl]- 5-phenyl-2H- pyrazole-3- carboxamide | 358 | 359 | 9.15 |
| 49 | 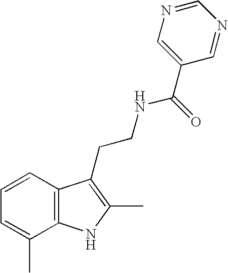 | N-[2-(2,7-dimethyl- 1H-indol-3-yl)ethyl]- pyrimidine-5- carboxamide | 294 | 295 | 7.48 |
| 50 | 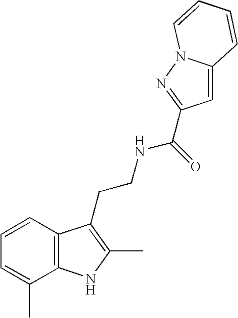 | N-[2-(2,7-dimethyl- 1H-indol-3-yl)ethyl]- pyrazolo[1,5- a]pyridine-2- carboxamide | 332 | 333 | 9.07 |
| 51 | 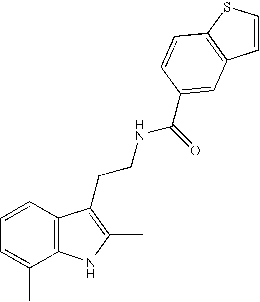 | N-[2-(2,7-dimethyl- 1H-indol-3-yl)ethyl]- benzo[b]thiophene- 5-carboxamide | 348 | 349 | 9.85 |
| 52 | 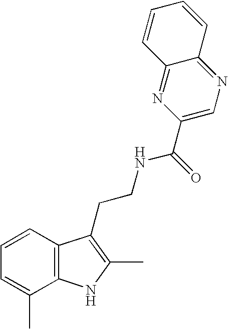 | N-[2-(2,7-dimethyl- 1H-indol-3-yl)ethyl]- quinoxaline-2- carboxamide | 344 | 345 | 9.86 |
| 53 | 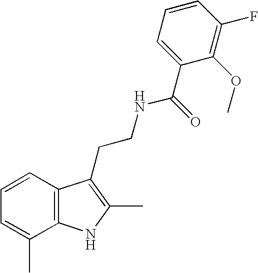 | N-[2-(2,7-dimethyl- 1H-indol-3-yl)ethyl]- 3-fluoro-2-methoxy- benzamide | 340 | 341 | 9.86 |
| 54 | 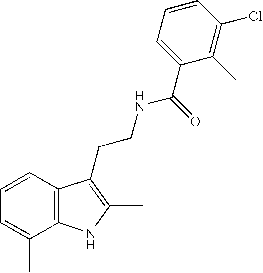 | 3-chloro-N-[2-(2,7- dimethyl-1H-indol-3- yl)ethyl]-2-methyl- benzamide | 340 | 341 | 9.98 |
| 55 | 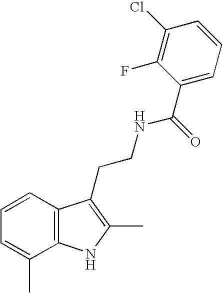 | 3-chloro-N-[2-(2,7- dimethyl-1H-indol-3- yl)ethyl]-2-fluoro- benzamide | 344 | 345 | 997 |
| 56 | 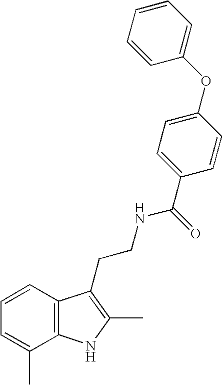 | N-[2-(2,7-dimethyl- 1H-indol-3-yl)ethyl]- 4-phenoxybenz- amide | 384 | 385 | 10.53 |
| 57 | 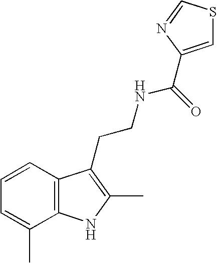 | N-[2-(2,7-dimethyl- 1H-indol-3-yl)ethyl]- thiazole-4- carboxamide | 299 | 300 | 8.6 |
| 58 | 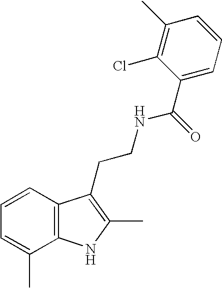 | 2-chloro-N-[2-(2,7- dimethyl-1H-indol-3- yl)ethyl]-3-methyl- benzamide | 340 | 341 | 9.7 |
| 59 | 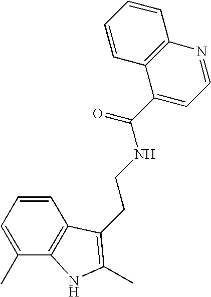 | N-[2-(2,7-dimethyl- 1H-indol-3-yl)ethyl]- quinoline-4- carboxamide | 343 | 344 | 7.08 |
| 60 | 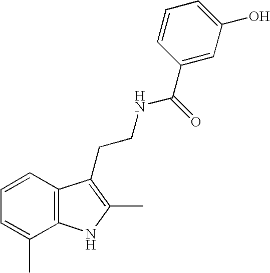 | N-[2-(2,7-dimethyl- 1H-indol-3-yl)ethyl]- 3-hydroxybenzamide | 308 | 309 | 8.11 |
| 61 | 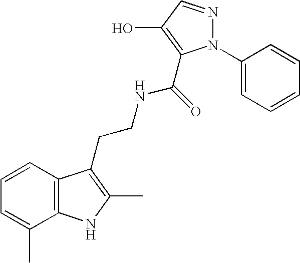 | N-[2-(2,7-dimethyl- 1H-indol-3-yl)ethyl]- 4-hydroxy-2-phenyl- 2H-pyrazole-3- carboxamide | 374 | 375 | 8.35 |
| 62 | 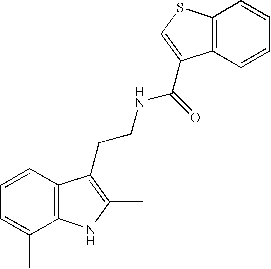 | N-[2-(2,7-dimethyl- 1H-indol-3-yl)ethyl]- benzo[b]thiophene- 3-carboxamide | 348 | 349 | 10.08 |
| 63 | 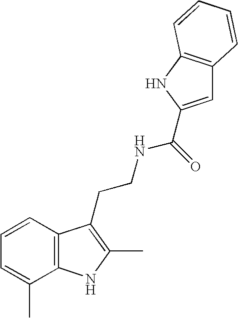 | N-[2-(2,7-dimethyl- 1H-indol-3-yl)ethyl]- 1H-indole-2- carboxamide | 331 | 332 | 9.55 |
| 64 | 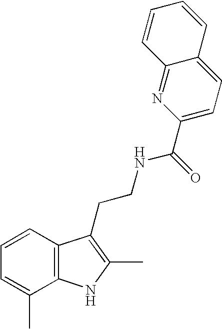 | N-[2-(2,7-dimethyl- 1H-indol-3-yl)ethyl]- quinoline-2- carboxamide | 343 | 344 | 10.43 |
| 65 | 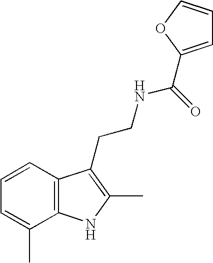 | N-[2-(2,7-dimethyl- 1H-indol-3-yl)ethyl]- furan-2-carboxamide | 282 | 283 | 8.55 |
| 66 | 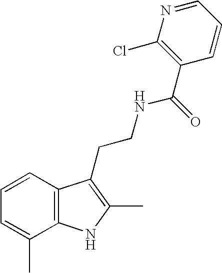 | 2-chloro-N-[2-(2,7- dimethyl-1H-indol-3- yl)ethyl]nicotinamide | 327 | 328 | 8.28 |
| 67 | 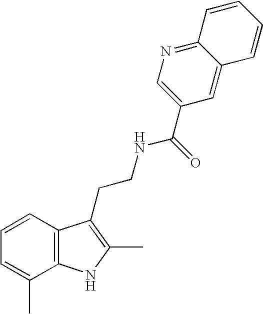 | N-[2-(2,7-dimethyl- 1H-indol-3-yl)ethyl]- quinoline-3- carboxamide | 343 | 344 | 7.78 |
| 68 | 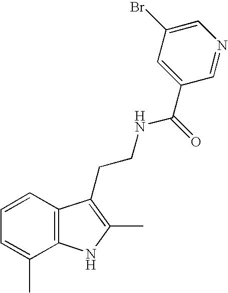 | 5-bromo-N-[2-(2,7- dimethyl-1H-indole-3- yl)ethyl]nicotinamide | 372 | 373 | 9.05 |
| 69 | 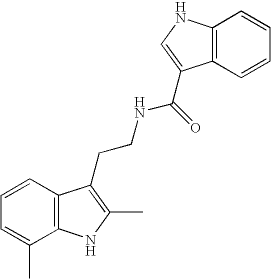 | N-[2-(2,7-dimethyl- 1H-indol-3-yl)ethyl]- 1H-indole-3- carboxamide | 331 | 332 | 8.73 |
| 70 | 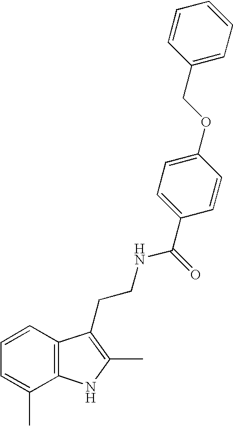 | 4-benzyloxy-N-[2- (2,7-dimethyl-1H- indol-3-yl)ethyl]- benzamide | 398 | 399 | 10.36 |
| 71 | 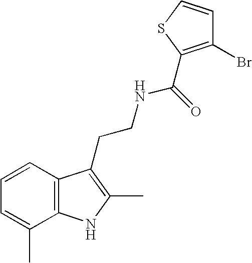 | N-[2-(2,7-dimethyl- 1H-indol-3-yl)ethyl]- 3-bromothiophene-2- carboxamide | 377 | 378 | 9.93 |
| 72 | 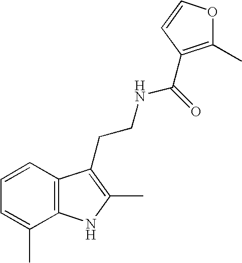 | N-[2-(2,7-dimethyl- 1H-indol-3-yl)ethyl]- 2-methylfuran-2- carboxamide | 296 | 297 | 9.06 |
| 73 | 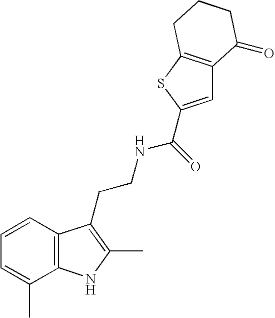 | N-[2-(2,7-dimethyl- 1H-indol-3-yl)ethyl]- 4-oxo-4,5,6,7- tetrahydrobenzo[b]- thiophene-2- carboxamide | 366 | 367 | 9.03 |
| 74 | 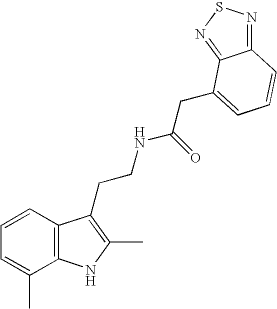 | 2-benzo[1,2,5]- thiadiazol-4-yl-N-[2- (2,7-dimethyl-1H- indol-3-yl)ethyl]- acetamide | 364 | 365 | 9.1 |
| 75 | 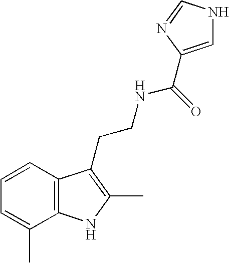 | N-[2-(2,7-dimethyl- 1H-indol-3-yl)ethyl]- 1H-imidazole-4- carboxamide | 282 | 283 | 6.1 |
| 76 | 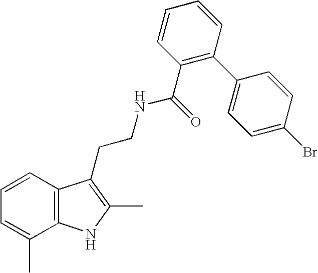 | N-[2-(2,7-dimethyl- 1H-indol-3-yl)ethyl]- 4′-bromobiphenyl-2- carboxamide | 447 | 448 | 10.64 |
| 77 | 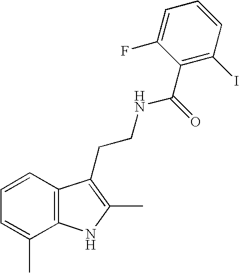 | N-[2-(2,7-dimethyl- 1H-indol-3-yl)ethyl]- 2-fluoro-6-iodobenz- amide | 436 | 437 | 9.52 |
| 78 | 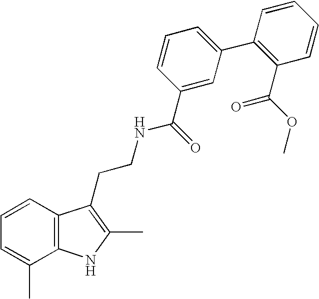 | 3′-[2-(2,7-dimethyl- 1H-indol-3-yl)ethyl- carbamoyl]biphenyl- 2-carboxylic acid methyl ester | 426 | 427 | 10.08 |
| 79 | 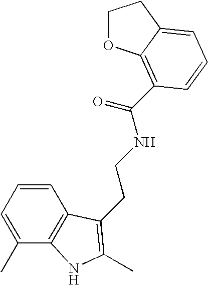 | N-[2-(2,7-dimethyl- 1H-indol-3-yl)ethyl]- 2,3-dihydrobenzo- furan-7-carboxamide | 334 | 335 | 9.57 |
| 80 | 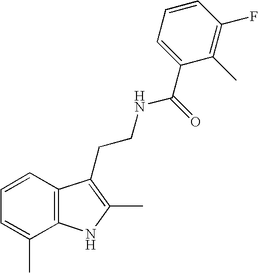 | N-[2-(2,7-dimethyl- 1H-indol-3-yl)ethyl]- 3-fluoro-2-methyl- benzamide | 324 | 325 | 9.52 |
| 81 | 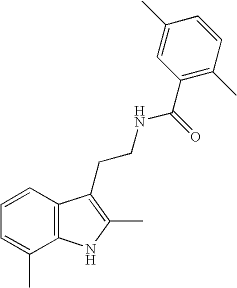 | N-[2-(2,7-dimethyl- 1H-indol-3-yl)ethyl]- 2,5-dimethylbenz- amide | 320 | 321 | 9.73 |
| 82 | 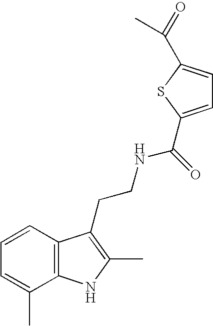 | N-[2-(2,7-dimethyl- 1H-indol-3-yl)ethyl]- 5-acetylthiophene-2- carboxamide | 340 | 341 | 8.86 |
| 83 | 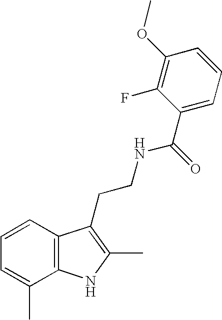 | N-[2-(2,7-dimethyl- 1H-indol-3-yl)ethyl]- 2-fluoro-3-methoxy- benzamide | 340 | 341 | 9.33 |
| 84 | 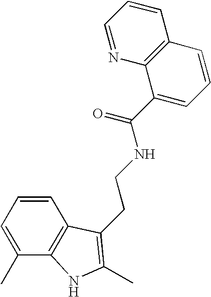 | N-[2-(2,7-dimethyl- 1H-indol-3-yl)ethyl]- quinoline-8- carboxamide | 343 | 344 | 8.53 |
| 85 | 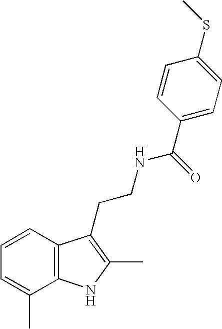 | N-[2-(2,7-dimethyl- 1H-indol-3-yl)ethyl]- 4-methylsulfanyl- benzamide | 338 | 339 | 9.63 |
| 86 | 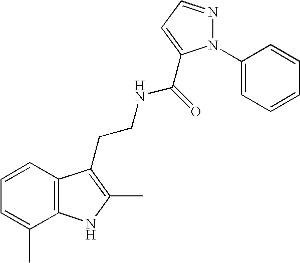 | N-[2-(2,7-dimethyl- 1H-indol-3-yl)ethyl]- 2-phenyl-2H- pyrazole-3- carboxamide | 358 | 359 | 9.1 |
| 87 | 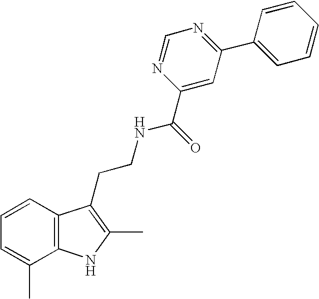 | N-[2-(2,7-dimethyl- 1H-indol-3-yl)ethyl]- 6-phenylpyrimidine- 4-carboxamide | 370 | 371 | 10.66 |
| 88 | 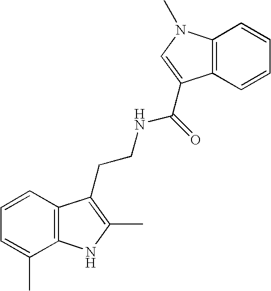 | N-[2-(2,7-dimethyl- 1H-indol-3-yl)ethyl]- 1-methyl-1H-indole- 3-carboxamide | 345 | 346 | 9.27 |
| 89 | 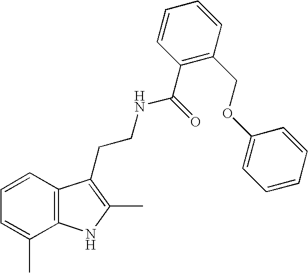 | N-[2-(2,7-dimethyl- 1H-indol-3-yl)ethyl]- 2-phenoxymethyl- benzamide | 398 | 399 | 10.42 |
| 90 | 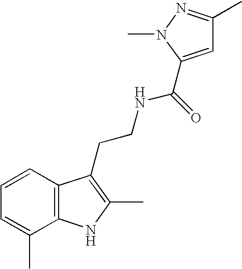 | N-[2-(2,7-dimethyl- 1H-indol-3-yl)ethyl]- 2,5-dimethyl-2H- pyrazole-3- carboxamide | 310 | 311 | 8.48 |
| 91 | 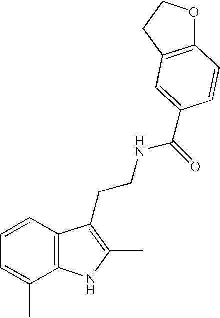 | N-[2-(2,7-dimethyl- 1H-indol-3-yl)ethyl]- 2,5-dihydrobenzo- furan-5-carboxamide | 334 | 335 | 9.05 |
| 92 | 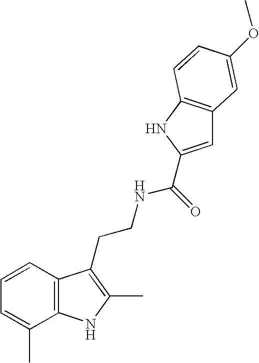 | N-[2-(2,7-dimethyl- 1H-indol-3-yl)ethyl]- 5-methoxy-1H- indole-2- carboxamide | 361 | 362 | 9.12 |
| 93 | 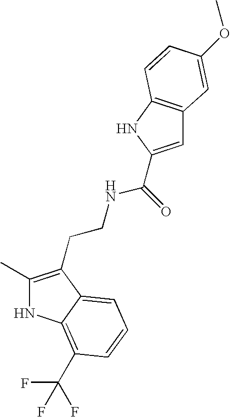 | N-[2-(2-methyl-7- trifluoromethyl-1H- indol-3-yl)ethyl]-5- methoxy-1H-indole- 2-carboxamide | 415 | 416 | 9.58 |
| 94 | 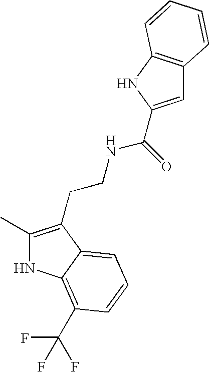 | N-[2-(2-methyl-7- trifluoromethyl-1H- indol-3-yl)ethyl]-1H- indole-2- carboxamide | 385 | 386 | 9.78 |
| 95 | 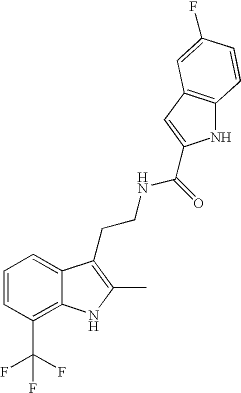 | N-[2-(2-methyl-7- trifluoromethyl-1H- indol-3-yl)ethyl]-5- fluoro-1H-indole-2- carboxamide | 403 | 404 | 9.97 |
| 96 | 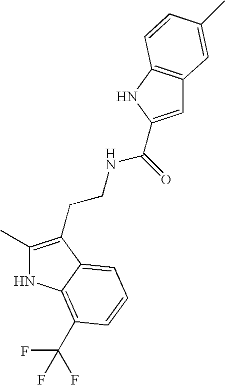 | N-[2-(2-methyl-7- trifluoromethyl-1H- indol-3-yl)ethyl]-5- methyl-1H-indole-2- carboxamide | 399 | 400 | 10.07 |
| 97 | 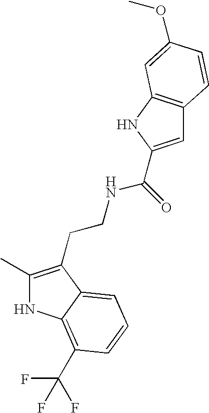 | N-[2-(2-methyl-7- trifluoromethyl-1H- indol-3-yl)ethyl]-6- methoxy-1H-indole- 2-carboxamide | 415 | 416 | 9.66 |
| 98 | 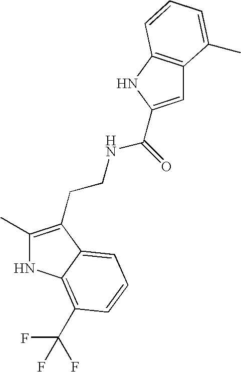 | N-[2-(2-methyl-7- trifluoromethyl-1H- indol-3-yl)ethyl]-4- methyl-1H-indole-2- carboxamide | 399 | 400 | 10.17 |
| 99 | 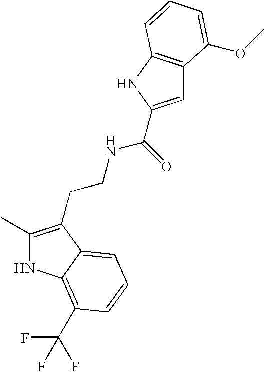 | N-[2-(2-methyl-7- trifluoromethyl-1H- indol-3-yl)ethyl]-4- methoxy-1H-indole- 2-carboxamide | 415 | 416 | 9.64 |
| 100 | 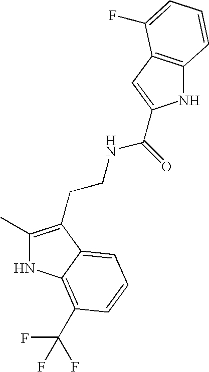 | N-[2-(2-methyl-7- trifluoromethyl-1H- indol-3-yl)ethyl]-4- fluoro-1H-indole-2- carboxamide | 403 | 404 | 9.98 |
| 101 | 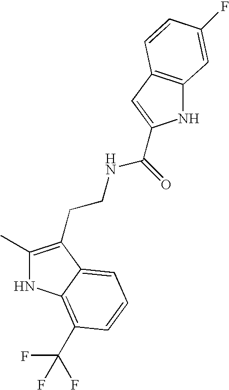 | N-[2-(2-methyl-7- trifluoromethyl-1H- indol-3-yl)ethyl]-6- fluoro-1H-indole-2- carboxamide | 403 | 404 | 9.89 |
| 102 | 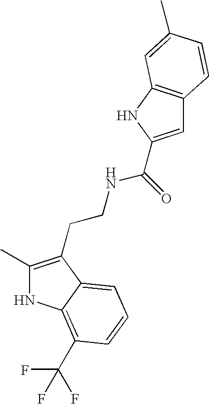 | N-[2-(2-methyl-7- trifluoromethyl-1H- indol-3-yl)ethyl]-6- methyl-1H-indole-2- carboxamide | 399 | 400 | 10.05 |
| 103 | 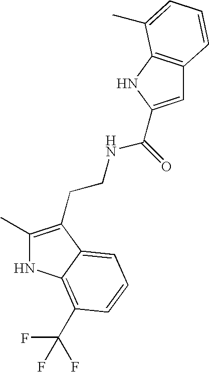 | N-[2-(2-methyl-7- trifluoromethyl-1H- indol-3-yl)ethyl]-7- methyl-1H-indole-2- carboxamide | 399 | 400 | 9.61 |
| 104 | 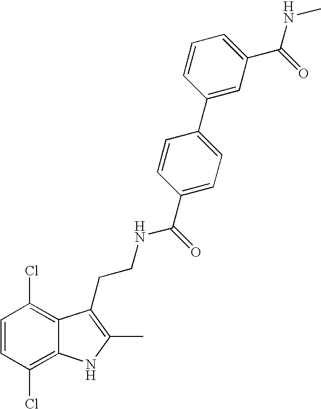 | 3-N-methyl-4′-N′-[2- (4,7-dichloro-2- methyl-1H-indol-3- yl)ethyl]biphenyl-3,4′- dicarboxamide | 480 | 481 | 3.99 |

### BIOLOGICAL EXAMPLES

1. Detection of the Antagonism of the Human Prostaglandin E₂ (Subtype EP₂) Receptor Signal

1.1 Principle of Detection

The binding of PGE₂ to the EP₂ subtype of the human PGE₂ receptor induces activation of membrane-associated adenylate cyclases and leads to the formation of cAMP. In the presence of the phosphodiesterase inhibitor IBMX, cAMP which has accumulated due to this stimulation and been released by cell lysis is employed in a competitive detection method. In this assay, the cAMP in the lysate competes with cAMP-XL665 for binding of an Eu cryptate-labelled anti-cAMP antibody.

This results, in the absence of cellular cAMP, in a maximum signal which derives from coupling of this antibody to the cAMP-XL665 molecule. After excitation at 337 nm, this results in a FRET (fluorescence resonance energy transfer)-based, long-lived emission signal at 665 nm (and at 620 nm). The two lo signals are measured in a suitable measuring instrument with a time lag, i.e. after the background fluorescence has declined. Any increase in the low FRET signal caused by prostaglandin E₂ addition (measured as well ratio change=emission_{665 nm}/emission_{620 nm}*10 000) shows the effect of antagonists.

1.2. Detection Method

1.2.1 Antagonism assay (data for each well of a 384-well plate):

The substance solutions (0.75 μl) introduced into an assay plate and 30% DMSO were dissolved in 16 μl of a KRSB+IBMX stimulation solution (1×Krebs-Ringer Bicarbonate Buffer; Sigma-Aldrich # K-4002; including 750 μM 3-isobutyl-1-methylxanthine Sigma-Aldrich # I-7018), and then 15 μl thereof were transferred into a media-free cell culture plate which had been washed with KRSB shortly beforehand.

After preincubation at room temperature (RT) for 30 minutes, 5 μl of a 4×PGE₂ solution (11 nM) were added, and incubation was carried out in the presence of the agonist at RT for a further 60 min (volume: ˜20 μl) before the reaction was then stopped by adding 5 μl of lysis buffer and incubated at RT for a further 20 min (volume: ˜25 μl). The cell lysate was then transferred into a measuring plate and measured in accordance with the manufacturer's information (cyclic AMP kit Cisbio International #62AMPPEC).

1.2.2 Agonism Assay (Data for each Well of a 384-Well Plate):

The substance solutions (0.75 μl) introduced into an assay plate and 30% DMSO were dissolved in 16 μl of a KRSB+IBMX stimulation solution (1×Krebs-Ringer Bicarbonate Buffer; Sigma-Aldrich # K-4002; including 750 μM 3-isobutyl-1-methylxanthine Sigma-Aldrich # I-7018), and then 15 μl thereof were transferred into a media-free cell culture plate which had been washed with KRSB shortly beforehand.

After incubation at room temperature (RT; volume: ˜15 μl) for 60 minutes, the reaction was then stopped by adding 5 μl of lysis buffer and incubated at RT for a further 20 min (volume: ˜20 μl). The cell lysate was then transferred into a measuring plate and measured in accordance with the manufacturer's instructions (cyclic AMP kit Cisbio International #62AMPPEC).

2. The EP₂ Subtype of the PGE₂ Receptor and the Preovulatory Cumulus Expansion

2.1. Background:

In the preovulatory antral follicle, the oocyte is surrounded by cumulus cells which form a dense ring of cells around the oocyte. After the LH peak (lutenizing hormone), a series of processes is activated and leads to a large morphological change in this ring of cells composed of cumulus cells. In this case, the cumulus cells form an extracellular matrix which leads to so-called cumulus expansion (Vanderhyden et al. Dev Biol. August 1990;140(2):307-317). This cumulus expansion is an important component of the ovulatory process and of the subsequent possibility of fertilization.

Prostaglandins, and here prostaglandin E₂, whose synthesis is induced by the LH peak, are of crucial importance in cumulus expansion. Prostanoid EP₂ knockout mice (Hizaki et al. Proc Natl Acad Sci USA. Aug. 31, 1999;96(18):10501-6.) show a markedly reduced cumulus expansion and severe subfertility, demonstrating the importance of the prostanoid EP₂ receptor for this process.

2.2 Cumulus Expansion Assay In Vitro

Folliculogenesis was induced in immature female mice (strain : CD1 (ICR) from Charles River) at an age of 14-18 days by a single dose (intraperitoneal) of 10 I.U. of PMSG (Pregnant Mare Serum Gonadotropin; Sigma G-4877, Lot 68H0909). 47-50 hours after the injection, the ovaries were removed and the cumulus-oocyte complexes were removed. The cumulus complex is not yet expanded at this stage.

The cumulus-oocyte complexes were then incubated with prostaglandin E₂ (PGE₂) (0.3 μM), vehicle control (ethanol) or test substances for 20-24 hours.

Medium: alpha-MEM medium with 0.1 mM IBMX, pyruvates (0.23 mM) glutamines (2 mM), pen/strep 100 IU/ml pen. and 100 μg/ml strep. and HSA (8 mg/ml). Cumulus expansion was then established through the division into four stages (according to Vanderhyden et al. Dev Biol. August 1990;140(2):307-317).

TABLE 1
Example of the biological activity of the inventive compounds
(measured by means of the cAMP antagonism test):

| Substance | |
|---|---|
| according to | |
| Example | Antagonism [IC₅₀, μM] |
| 1 | 0.32 |
| 5 | 1.9 |
| 7 | 0.047 |
| 8 | 0.12 |
| 17 | 0.72 |
| 30 | 0.67 |

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding EP application No. 08161438.0, filed Jul. 30, 2008, and U.S. Provisional Application Ser. No. 61/084,725 filed Jul. 30, 2008 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Compounds of the general formula I where 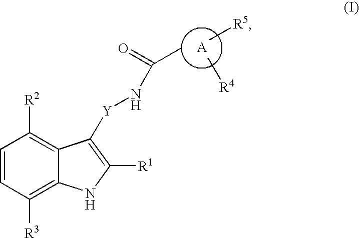
in which
A is a C₆-C₁₂-aryl or C₅-C₁₂-heteroaryl radical which may optionally be mono- or polysubstituted by R⁴ and/or R⁵,
R¹ is a C₁-C₆-alkyl radical which may optionally be substituted,
R² is a hydrogen, halogen, cyano, an —S(O)_{q}—CH₃ where q is 0-2, a C₁-C₄-alkoxy radical or C₁-C₆-alkyl, where this radical may be substituted as desired,
R³ is a hydrogen, a C₁-C₆-alkyl radical, cyano, chlorine or bromine,
R⁴ is a hydrogen, halogen, amino, an —SO(O)ₚ—C₁-C₆-alkyl group where p is 0-2,
a C₁-C₆-acyl, NH—CO—NH₂, —O—CO—NH(C₁-C₆-alkyl), —O—CO—N(C₁-C₆-alkyl)₂ or NH—CO—C₁-C₆-alkyl radical,
a C₁-C₆-alkyl which may optionally be mono- or polysubstituted, identically or differently, by C₁-C₆-acyl, C₁-C₆-alkoxy, hydroxyl, cyano, CO₂—(C₁-C₆-alkyl), N—(C₁-C₆-alkyl)₂, C₅-C₁₂-heteroaryl, COOH, CO—NH₂, CO—NH(C₁-C₆-alkyl) or by CO—N(C₁-C₆-alkyl)₂,
a C₁-C₆-alkoxy which may optionally be mono- or polysubstituted, identically or differently, by hydroxyl, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or by CO—N(C₁-C₆-alkyl)₂,
an O—C₆-C₁₂-aryl which may optionally be substituted by hydroxyl, cyano, COOH or CO—NH₂,
a CH₂O—C₆-C₁₂-aryl which may optionally be substituted by hydroxyl, cyano, COOH or CO—NH₂,
an O—C₅-C₁₆-heteroaryl which may optionally be substituted by hydroxyl, cyano, COOH or CO—NH₂,
a hydroxyl, cyano, O—CO—(C₁-C₆-alkyl), CO—NH(C₅-C₁₂-heteroaryl), NH—(C₁-C₆-alkyl), N—(C₁-C₆-alkyl)₂,
a C₆-C₁₂-aryl which may optionally be mono- or polysubstituted, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxyl, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or by —S(O)ᵣ—CH₃ where r is 0-2, or two adjacent positions may be substituted by —O—CH₂—O— or —O—C(CH₃)₂—O—,
a monocyclic C₅-C₇-heteroaryl which may optionally be mono- or polysubstituted, identically or differently, by C₁-C₆-alkyl,
provided that R² is cyano or R¹ and/or R² are the same or different and are each a C₁-C₆-alkyl radical, where at least one of the radicals is at least monosubstituted or
provided that R⁵ is an —SO(O)ₚ—C₁-C₆-alkyl where p is 0-2, is a C₁-C₆-acyl, —O—CO—NH(C₁-C₆-alkyl), —O—CO—N(C₁-C₆-alkyl)₂, C₆-C₁₂-aryloxy, C₅-C₁₆-heteroaryloxy, hydroxyl, cyano or N—(C₁-C₆-alkyl)₂,
a monocyclic C₅-C₇-heteroaryl which may be at least mono- or polysubstituted, identically or differently, by halogen, CF₃, C₁-C₆-acyl, C₁-C₆-alkoxy, hydroxyl, CH₂—OH, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂,
a bi- or tricyclic C₈-C₁₂-heteroaryl which may optionally be mono- or polysubstituted, identically or differently, by halogen, by C₁-C₆-alkyl, C₁-C₆-acyl, C₁-C₆-alkoxy, hydroxyl, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂ or
a C₃-C₆-cycloalkyl which may optionally be mono- or polysubstituted, identically or differently, by halogen, by C₁-C₆-alkyl, hydroxyl, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R⁵ is a hydrogen, halogen, amino, —SO(O)ₚ—C₁-C₆-alkyl where p is 0-2,
a C₁-C₆-acyl, NH—CO—NH₂, NH—CO—C₁-C₆-alkyl, —O—CO—NH(C₁-C₆-alkyl), —O—CO—N(C₁-C₆-alkyl)₂ or C₁-C₆-alkyl group which may optionally be mono- or polysubstituted, identically or differently, by C₁-C₆-acyl, C₁-C₆-alkoxy, hydroxyl, cyano, CO₂—(C₁-C₆-alkyl), N—(C₁-C₆-alkyl)₂, C₅-C₁₂-heteroaryl, COOH, CO—NH₂, CO—NH(C₁-C₆-alkyl) or by CO—N(C₁-C₆-alkyl)₂,
a C₁-C₆-alkoxy which may optionally be mono- or polysubstituted, identically or differently, by hydroxyl, cyano, CO₂—(C₁-C₆-alkyl), N—(C₁-C₆-alkyl)₂, NH—C₃-C₆-cycloalkyl, COOH, CO—NH₂, CO—NH(C₁-C₆-alkyl) or by CO—N(C₁-C₆-alkyl)₂,
an O—C₆-C₁₂-aryl which may optionally be substituted by hydroxyl, cyano, COOH or CO—NH₂,
a CH₂O—C₆-C₁₂-aryl which may optionally be substituted by hydroxyl, cyano, COOH or CO—NH₂,
an O—C₅-C₁₆-heteroaryl which may optionally be substituted by hydroxyl, cyano, COOH or CO—NH₂,
a hydroxyl, cyano, O—CO—(C₁-C₆-alkyl), CO—NH(C₅-C₁₂-heteroaryl), NH—(C₁-C₆-alkyl), N—(C₁-C₆-alkyl)₂,
a C₆-C₁₂-aryl which may optionally be mono- or polysubstituted, identically or differently, by halogen, by C₁-C₆-alkyl, C₃-C₆-cycloalkyl, C₁-C₆-acyl, C₁-C₆-alkoxy, C₆-C₁₂-aryl, C₅-C₁₂-heteroaryl, hydroxyl, CH₂—OH, cyano, CH₂—CN, amino, CO₂—(C₁-C₆-alkyl), N—(C₁-C₆-alkyl)₂, NHSO₂CH₃, SO₂NH₂, SO₂NH(C₁-C₆-alkyl), SO₂N(C₁-C₆-alkyl)₂, COOH, CO—NH₂, CO—NH(C₁-C₆-alkyl), CO—N(C₁-C₆-alkyl)₂, CO—NH(C₅-C₁₂-heteroaryl), NH—CO(C₁-C₆-alkyl), CH₂—NH—CO(C₁-C₆-alkyl), NH—CO(C₅-C₁₂-heteroaryl), CH₂—NH—CO(C₅-C₁₂-heteroaryl), styryl, or by —S(O)ᵣ—CH₃ where r is 0-2, or two adjacent positions may be substituted by —O—CH₂—O—or —O—C(CH₃)₂—O—,
a monocyclic C₅-C₇-heteroaryl which may optionally be mono- or polysubstituted, identically or differently, by C₁-C₆-alkyl when R² is cyano, or provided that R¹ and/or R² are the same or different and are each a C₁-C₆-alkyl radical, where at least one of the radicals is at least monosubstituted,
a monocyclic C₅-C₇-heteroaryl which may be at least mono- or polysubstituted, identically or differently, by halogen, CF₃, C₁-C₆-acyl, C₁-C₆-alkoxy, hydroxyl, CH₂—OH, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂,
a bi- or tricyclic C₈-C₁₂-heteroaryl which may optionally be mono- or polysubstituted, identically or differently, by halogen, by C₁-C₆-alkyl, C₁-C₆-acyl, C₁-C₆-alkoxy, hydroxyl, cyano, CO₂—(C₁-C₆-alkyl), N—(C₁-C₆-alkyl)₂, COOH, CO—NH₂, CO—NH(C₁-C₆-alkyl) or CO—N(C₁-C₆-alkyl)₂ or
a C₃-C₆-cycloalkyl which may optionally be mono- or polysubstituted, identically or differently, by halogen, by C₁-C₆-alkyl, hydroxyl, cyano, CO₂—(C₁-C₆-alkyl), C₁-C₆-acyl, N—(C₁-C₆-alkyl)₂, COOH, CO—NH₂, CO—NH(C₁-C₆-alkyl), CO—N(C₁-C₆-alkyl)₂ or C₁-C₆-alkoxy,
R⁴ and R⁵ are in ortho, meta or meta,para positions with respect to one another and together are defined as —O—CO—S—, —S—CO—O—, CH₂-CO—O—, O—CO—CH₂—, —CH₂—CO—NH—, —NH—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂— where m is 1-3,
Y is a —(CH₂)ₙ group where n is 1-3,
and the isomers, diastereomers, enantiomers and salts or cyclodextrin clathrates thereof, excluding the following compounds:
4-chloro-1,3-dimethyl-N-[2-(2-methyl-1H-indol-3-yl)ethyl]-1H-pyrazole-5-carboxamide
N-[2-(2-methyl-1H-indol-3-yl)ethyl]-6-quinoxalinecarboxamide
3,5-dimethyl-N-[2-(2-methyl-1H-indol-3-yl)ethyl]-4-isoxazolecarboxamide
5,7-dimethyl-N-[2-(2-methyl-1H-indol-3-yl)ethyl]pyrazolo[1,5-a]pyrimidine-2-carboxamide
N-[2-(2-methyl-1H-indol-3-yl)ethyl]-2-methylbenzamide
N-[2-(2-methyl-1H-indol-3-yl)ethyl]-2-(difluoromethyl)benzamide
3-iodo-1-methyl-N-[2-(2-methyl-1H-indol-3-yl)ethyl]-1H-pyrrole-2-carboxamide
1-methyl-N-[2-(2-methyl-1H-indol-3-yl)ethyl]-4-(trifluoromethyl)-1H-pyrrole-3-carboxamide
2-iodo-N-[2-(2-methyl-1H-indol-3-yl)ethyl]-3-thiophenecarboxamide
3-(difluoromethyl)-1-methyl-N-[2-(2-methyl-1H-indol-3-yl)ethyl]-1H-pyrazole-4-carboxamide
3-(trifluoromethyl)-1-methyl-N-[2-(2-methyl-1H-indol-3-yl)ethyl]-1H-pyrazole-4-carboxamide
5-fluoro-1,3-dimethyl-N-[2-(2-methyl-1H-indol-3-yl)ethyl]-1H-pyrazole-4-carboxamide
4-(difluoromethyl)-2-methyl-N-[2-(2-methyl-1H-indol-3-yl)ethyl]-5-thiazolecarboxamide
4-(trifluoromethyl)-2-methyl-N-[2-(2-methyl-1H-indol-3-yl)ethyl]-5-thiazolecarboxamide
2-iodo-N-[2-(2-methyl-1H-indol-3-yl)ethyl]-3-furancarboxamide
2,5-dimethyl-N-[2-(2-methyl-1H-indol-3-yl)ethyl]-3-furancarboxamide
5-methyl-N-[2-(2-methyl-1H-indol-3-yl)ethyl]-2-(trifluoromethyl)-3-furancarboxamide
2-methyl-N-[2-(2-methyl-1H-indol-3-yl)ethyl]-3-furancarboxamide
3-methyl-N-[2-(2-methyl-1H-indol-3-yl)ethyl]-2-thiophenecarboxamide
3-iodo-N-[2-(2-methyl-1H-indol-3-yl)ethyl]-2-thiophenecarboxamide
3-bromo-N-[2-(2-methyl-1H-indol-3-yl)ethyl]-2-thiophenecarboxamide
4-methoxy-N-[2-(2-methyl-1H-indol-3-yl)ethyl]benzamide
N-[2-(7-chloro-2-methyl-1H-indol-3-yl)ethyl]-2-methyl-7-benzoxazolecarboxamide
4-(1,1-dimethylethyl)-N-[2-(2-methyl-1H-indol-3-yl)ethyl]benzamide
N-[2-(2,7-dimethyl-1H-indol-3-yl)ethyl]-3,4-dimethoxybenzamide
2-ethoxy-N-[2-(2-methyl-1H-indol-3-yl)ethyl]benzamide
2-(1-methylethoxy)-N-[2-(2-methyl-1H-indol-3-yl)ethyl]benzamide
2-chloro-4,5-difluoro-N-[2-(2-methyl-1H-indol-3-yl)ethyl]benzamide
4-bromo-N-[2-(4-fluoro-2,7-dimethyl-1H-indol-3-yl)ethyl]benzamide
2,6-difluoro-N-[2-(2-methyl-1H-indol-3-yl)ethyl]benzamide
3,4-difluoro-N-[2-(2-methyl-1H-indol-3-yl)ethyl]benzamide
N-[2-(2-methyl-1H-indol-3-yl)ethyl]-4-(trifluoromethyl)benzamide
N-[2-(2-methyl-1H-indol-3-yl)ethyl]-4-methylbenzamide
2,4-dichloro-N-[2-(2-methyl-1H-indol-3-yl)ethyl]benzamide
2-bromo-N-[2-(2-methyl-1H-indol-3-yl)ethyl]benzamide
4-fluoro-N-[2-(2-methyl-1H-indol-3-yl)ethyl]benzamide
N-[2-(4-fluoro-2,7-dimethyl-1H-indol-3-yl)ethyl]-2-methylbenzamide
N-[2-(4-fluoro-2,7-dimethyl-1H-indol-3-yl)ethyl]-2-methoxybenzamide
3,4-dimethoxy-N-[2-(4-fluoro-2,7-dimethyl-1H-indol-3-yl)ethyl]benzamide
4-[[[2-(4-fluoro-2,7-dimethyl-1H-indol-3-yl)ethyl]amino]carbonyl]benzoic acid methyl ester
N-[2-(4-fluoro-2,7-dimethyl-1H-indol-3-yl)ethyl]-1-methyl-1H-pyrazole-5-carboxamide
N-[2-(7-ethyl-2-methyl-1H-indol-3-yl)ethyl]-2-methoxybenzamide
4-chloro-N-[2-(2-methyl-1H-indol-3-yl)ethyl]benzamide
2,6-dichloro-N-[2-(2-methyl-1H-indol-3-yl)ethyl]benzamide
5-chloro-2-methoxy-N-[2-(2-methyl-1H-indol-3-yl)ethyl]benzamide
3,4-dichloro-N-[2-(2-methyl-1H-indol-3-yl)ethyl]benzamide
2-fluoro-N-[2-(2-methyl-1H-indol-3-yl)ethyl]benzamide
4-chloro-1-methyl-N-[2-(2-methyl-1H-indol-3-yl)ethyl]-1H-pyrazole-5-carboxamide
4-[[[2-(2-methyl-1H-indol-3-yl)ethyl]amino]carbonyl]-benzoic acid methyl ester
N-[2-(2-methyl-1H-indol-3-yl)ethyl]-2-thiophenecarboxamide
3,4,5-trimethoxy-N-[2-(2-methyl-1H-indol-3-yl)ethyl]benzamide
3-methoxy-N-[2-(2-methyl-1H-indol-3-yl)ethyl]benzamide
N-[2-(2-methyl-1H-indol-3-yl)ethyl]-4-pyridinecarboxamide
N-[2-(2-methyl-1H-indol-3-yl)ethyl]-3-pyridinecarboxamide

2. Compounds according to claim 1, where
A is a C₆-C₁₂-aryl or C₅-C₁₂-heteroaryl radical which may optionally be mono- or polysubstituted by R⁴ and/or R⁵,
R¹ is a C₁-C₄-alkyl radical which may optionally be substituted,
R² is a hydrogen, halogen, a C₁-C₄-alkoxy radical or C₁-C₆-alkyl, where this radical may be substituted as desired,
R³ is a hydrogen, a C₁-C₆-alkyl radical, cyano, chlorine or bromine, and Y are each as defined in claim 1,
and the isomers, diastereomers, enantiomers and salts or cyclodextrin clathrates thereof.

3. Compounds according to claim 1, where
A is a C₆-C₁₂-aryl or C₅-C₁₂-or heteroaryl radical which may optionally be mono- or polysubstituted by R⁴ and/or R⁵,
R¹ is a C₁-C₄-alkyl radical,
R² is a hydrogen, halogen or C₁-C₄-alkyl, where this radical may be substituted as desired,
R³ may be a hydrogen, a C₁-C₄-alkyl radical, cyano, chlorine or bromine,
R⁴ is a hydrogen, halogen, amino, an —SO(O)ₚ—C₁-C₄-alkyl group where p is 0-2,
a C₁-C₄-acyl, NH—CO—NH₂, —O—CO—NH(C₁-C₄-alkyl), —O—CO—N(C₁-C₄-alkyl)₂ or NH—CO—C₁-C₄-alkyl radical,
a C₁-C₄-alkyl which may optionally be mono- or polysubstituted, identically or differently, by C₁-C₄-acyl, C₁-C₄-alkoxy, hydroxyl, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
a C₁-C₄-alkoxy which may optionally be mono- or polysubstituted, identically or differently, by hydroxyl, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂, an O—C₆-C₁₂-aryl which may optionally be substituted by hydroxyl, cyano, COOH or CO—NH₂,
a CH₂O—C₆-C₁₂-aryl which may optionally be substituted by hydroxyl, cyano, COOH or CO—NH₂,
an O—C₅-C₁₆-heteroaryl which may optionally be substituted by hydroxyl, cyano, COOH or CO—NH₂,
a hydroxyl, cyano, O—CO—(C₁-C₄-alkyl), CO—NH(C₅-C₁₂-heteroaryl), NH—(C₁-C₄-alkyl), N—(C₁-C₄-alkyl)₂,
a C₆-C₁₂-aryl which may optionally be mono- or polysubstituted, identically or differently, by halogen, by C₁-C₄-alkyl, C₃-C₆-cycloalkyl, C₁-C₄-acyl, C₁-C₄-alkoxy, C₆-C₁₂-aryl, C₅-C₁₂-heteroaryl, hydroxyl, CH₂—OH, cyano, CH₂—CN, amino, CO₂—(C₁-C₄-alkyl), N—(C₁-C₄-alkyl)₂, NHSO₂CH₃, SO₂NH₂, SO₂NH(C₁-C₄-alkyl), SO₂N(C₁-C₄-alkyl)₂, COOH, CO—NH₂, CO—NH(C₁-C₄-alkyl), CO—N(C₁-C₄-alkyl)₂, CO—NH(C₅-C₁₂-heteroaryl), NH—CO(C₁-C₄-alkyl), CH₂—NH—CO(C₁-C₄-alkyl), NH—CO(C₅-C₁₂-heteroaryl), CH₂—NH—CO(C₅-C₁₂-heteroaryl), styryl, or by —S(O)ᵣ—CH₃ where r is 0-2, or two adjacent positions may be substituted by —O—CH₂—O— or —O—C(CH₃)₂—O—,
a monocyclic C₅-C₇-heteroaryl which may optionally be mono- or polysubstituted, identically or differently, by C₁-C₄-alkyl,
provided that R² cyano or R¹ and/or R² are the same or different and are each a C₁-C₄-alkyl radical, where at least one of the radicals is at least monosubstituted or provided that R⁵ is an —SO(O)ₚ—C₁-C₄-alkyl where p is 0-2, is a C₁-C₆-acyl, —O—CO—NH(C₁-C₄-alkyl), —O—CO—N(C₁-C₄-alkyl)₂, C₆-C₁₂-aryloxy, C₅-C₁₆-heteroaryloxy, hydroxyl, cyano or N—(C₁-C₄-alkyl)₂,
a monocyclic C₅-C₇-heteroaryl which may be at least mono- or polysubstituted, identically or differently, by halogen, CF₃, C₁-C₄-acyl, C₁-C₄-alkoxy, hydroxyl, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂,
a bi- or tricyclic C₈-C₁₂-heteroaryl which may optionally be mono- or polysubstituted, identically or differently, by halogen, by C₁-C₄-alkyl, C₁-C₄-acyl, C₁-C₄-alkoxy, hydroxyl, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂ or
a C₃-C₆-cycloalkyl which may optionally be mono- or polysubstituted, identically or differently, by halogen, by C₁-C₄-alkyl, hydroxyl, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C_{-C}₄-alkyl)₂ or C₁-C₄-alkoxy,
R⁵ is a hydrogen, halogen, amino, —SO(O)ₚ—C₁-C₄-alkyl where p is 0-2,
a C₁-C₄-acyl, NH—CO—NH₂—, NH—CO—C₁-C₄-alkyl-, —O—CO—NH(C₁-C₄-alkyl)-, —O—CO—N(C₁-C₄-alkyl)₂ or C₁-C₄-alkyl group which may optionally be mono- or polysubstituted, identically or differently, by C₁-C₄-acyl, C₁-C₄-alkoxy, hydroxyl, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
a C₁-C₄-alkoxy which may optionally be mono- or polysubstituted, identically or differently, by hydroxyl, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
an O—C₆-C₁₂-aryl which may optionally be substituted by hydroxyl, cyano, COOH or CO—NH₂,
a CH₂O—C₆-C₁₂-aryl which may optionally be substituted by hydroxyl, cyano, COOH or CO—NH₂,
an O—C₅-C₁₆-heteroaryl which may optionally be substituted by hydroxyl, cyano, COOH or CO—NH₂,
a hydroxyl, cyano, O—CO—(C₁-C₄-alkyl), CO—NH(C₅-C₁₂-heteroaryl), NH—(C₁-C₄-alkyl), N—(C₁-C₄-alkyl)₂,
a C₆-C₁₂-aryl which may optionally be mono- or polysubstituted, identically or differently, by halogen, by C₁-C₄-alkyl, C₃-C₆-cycloalkyl, C₁-C₄-acyl, C₁-C₄-alkoxy, C₆-C₁₂-aryl, C₅-C₁₂-heteroaryl, hydroxyl, CH₂—OH, cyano, CH₂—CN, amino, CO₂—(C₁-C₄-alkyl), N—(C₁-C₄-alkyl)₂, NHSO₂CH₃, SO₂NH₂, SO₂NH(C₁-C₄-alkyl), SO₂N(C₁-C₄-alkyl)₂, COOH, CO—NH₂, CO—NH(C₁-C₄-alkyl), CO—N(C₁-C₄-alkyl)₂, CO—NH(C₅-C₁₂-heteroaryl), NH—CO(C₁-C₄-alkyl), CH₂—NH—CO(C₁-C₄-alkyl), NH—CO(C₅-C₁₂-heteroaryl), CH₂—NH—CO(C₅-C₁₂-heteroaryl), styryl, or an —S(O)ᵣ—CH₃ where r is 0-2, or two adjacent positions may be substituted by —O—CH₂—O—or —O—C(CH₃)₂—O—,
a monocyclic C₅-C₇-heteroaryl which may optionally be mono- or polysubstituted, identically or differently, by C₁-C₄-alkyl, provided that R2 is cyano or when R¹ and/or R² is the same or different and is a C₁-C₄-alkyl radical, where at least one of the radicals is at least monosubstituted,
a monocyclic C₅-C₇-heteroaryl which may be at least mono- or polysubstituted, identically or differently, by halogen, CF₃, C₁-C₄-acyl, C₁-C₄-alkoxy, hydroxyl, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂,
a bi- or tricyclic C₈-C₁₂-heteroaryl which may optionally be mono- or polysubstituted, identically or differently, by halogen, by C₁-C₄-alkyl, C₁-C₄-acyl, C₁-C₄-alkoxy, hydroxyl, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂ or
a C₃-C₆-cycloalkyl which may optionally be mono- or polysubstituted, identically or differently, by halogen, by C₁-C₄-alkyl, hydroxyl, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R⁴ and R⁵ are in ortho, meta or meta,para positions with respect to one another and together are defined as —O—CO—S—, —S—CO—O—, —CH₂-CO—O—, —O—CO—CH₂—, —CH₂—CO—NH—, —NH—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂— where m is 1-3,
Y is a —(CH₂)ₙ group where n is 1-3,
and the isomers, diastereomers, enantiomers and salts or cyclodextrin clathrates thereof.

4. Compounds according to claim 1, where
A is a phenyl, naphthyl or heteroaryl radical which may optionally be mono- or disubstituted by R⁴ and/or R⁵,
R¹ is a C₁-C₄-alkyl radical,
R² is a hydrogen, halogen or C₁-C₄-alkyl, where this radical may be substituted as desired,
R³ is a hydrogen, a C₁-C₄-alkyl radical, cyano, chlorine or bromine
R⁴ is a hydrogen, halogen, amino, an —SO(O)ₚ—C₁-C₄-alkyl group where p is 0-2,
a C₁-C₄-acyl, NH—CO—NH₂, —O—CO—NH(C₁-C₄-alkyl), —O—CO—N(C₁-C₄-alkyl)₂ or NH—CO—C₁-C₄-alkyl radical,
a C₁-C₄-alkyl which may optionally be mono- or polysubstituted, identically or differently, by C₁-C₄-acyl, C₁-C₄-alkoxy, hydroxyl, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
a C₁-C₄-alkoxy which may optionally be mono- or polysubstituted, identically or differently, by hydroxyl, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂, an O—C₆-C₁₂-aryl which may optionally be substituted by hydroxyl, cyano, COOH or CO—NH₂,
a CH₂O—C₆-C₁₂-aryl which may optionally be substituted by hydroxyl, cyano, COOH or CO—NH₂,
an O—C₅-C₁₆-heteroaryl which may optionally be substituted by hydroxyl, cyano, COOH or CO—NH₂,
a hydroxyl, cyano, O—CO—(C₁-C₄-alkyl), CO—NH(C₅-C₁₂-heteroaryl), NH—(C₁-C₄-alkyl), N—(C₁-C₄-alkyl)₂,
a C₆-C₁₂-aryl which may optionally be mono- or polysubstituted, identically or differently, by halogen, by C₁-C₄-alkyl, C₃-C₆-cycloalkyl, C₁-C₄-acyl, C₁-C₄-alkoxy, C₆-C₁₂-aryl, C₅-C₁₂-heteroaryl, hydroxyl, CH₂—OH, cyano, CH₂—CN, amino, CO₂—(C₁-C₄-alkyl), N—(C₁-C₄-alkyl)₂, NHSO₂CH₃, SO₂NH₂, SO₂NH(C₁-C₄-alkyl), SO₂N(C₁-C₄-alkyl)₂, COOH, CO—NH₂, CO—NH(C₁-C₄-alkyl), CO—N(C₁-C₄-alkyl)₂, CO—NH(C₅-C₁₂-heteroaryl), NH—CO(C₁-C₄-alkyl), CH₂—NH—CO(C₁-C₄-alkyl), NH—CO(C₅-C₁₂-heteroaryl), CH₂—NH—CO(C₅-C₁₂-heteroaryl), styryl, or by —S(O)ᵣ—CH₃ where r is 0-2, or two adjacent positions may be substituted by —O—CH₂—O—or —O—C(CH₃)₂—O—,
a monocyclic C₅-C₇-heteroaryl which may optionally be mono- or polysubstituted, identically or differently, by C₁-C₄-alkyl, provided that R² cyano or R¹ and/or R² are the same or different and are each a C₁-C₄-alkyl radical, where at least one of the radicals is at least monosubstituted or
provided that R⁵ is an —SO(O)ₚ—C₁-C₄-alkyl where p is 0-2, is a C₁-C₆-acyl, —O—CO—NH(C₁-C₄-alkyl), —O—CO—N(C₁-C₄-alkyl)₂, C₆-C₁₂-aryloxy, C₅-C₁₆-heteroaryloxy, hydroxyl, cyano or N—(C₁-C₄-alkyl)₂,
a monocyclic C₅-C₇-heteroaryl which may be at least mono- or polysubstituted, identically or differently, by halogen, CF₃, C₁-C₄-acyl, C₁-C₄-alkoxy, hydroxyl, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂,
a bi- or tricyclic C₈-C₁₂-heteroaryl which may optionally be mono- or polysubstituted, identically or differently, by halogen, by C₁-C₄-alkyl, C₁-C₄-acyl, C₁-C₄-alkoxy, hydroxyl, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂ or
a C₃-C₆-cycloalkyl which may optionally be mono- or polysubstituted, identically or differently, by halogen, by C₁-C₄-alkyl, hydroxyl, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R⁵ is a hydrogen, halogen, amino, —SO(O)ₚ—C₁-C₄-alkyl where p is 0-2,
a C₁-C₄-acyl, NH—CO—NH₂—, NH—CO—C₁-C₄-alkyl-, —O—CO—NH(C₁-C₄-alkyl)-, —O—CO—N(C₁-C₄-alkyl)₂ or C₁-C₄-alkyl group which may optionally be mono- or polysubstituted, identically or differently, by C₁-C₄-acyl, C₁-C₄-alkoxy, hydroxyl, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
a C₁-C₄-alkoxy which may optionally be mono- or polysubstituted, identically or differently, by hydroxyl, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
an O—C₆-C₁₂-aryl which may optionally be substituted by hydroxyl, cyano, COOH or CO—NH₂,
a CH₂O—C₆-C₁₂-aryl which may optionally be substituted by hydroxyl, cyano, COOH or CO—NH₂,
an O—C₅-C₁₆-heteroaryl which may optionally be substituted by hydroxyl, cyano, COOH or CO—NH₂,
a hydroxyl, cyano, O—CO—(C₁-C₄-alkyl), CO—NH(C₅-C₁₂-heteroaryl), NH—(C₁-C₄-alkyl), N—(C₁-C₄-alkyl)₂,
a C₆-C₁₂-aryl which may optionally be mono- or polysubstituted, identically or differently, by halogen, by C₁-C₄-alkyl, C₃-C₆-cycloalkyl, C₁-C₄-acyl, C₁-C₄-alkoxy, C₆-C₁₂-aryl, C₅-C₁₂-heteroaryl, hydroxyl, CH₂—OH, cyano, CH₂—CN, amino, CO₂—(C₁-C₄-alkyl), N—(C₁-C₄-alkyl)₂, NHSO₂CH₃, SO₂NH₂, SO₂NH(C₁-C₁-C₄-alkyl), SO₂N(C₁-C₄-alkyl)₂, COOH, CO—NH₂, CO—NH(C₁-C₄-alkyl), CO—N(C₁-C₄-alkyl)₂, CO—NH(C₅-C₁₂-heteroaryl), NH—CO(C₁-C₄-alkyl), CH₂—NH—CO(C₁-C₄-alkyl), NH—CO(C₅-C₁₂-heteroaryl), CH₂—NH—CO(C₅-C₁₂-heteroaryl), styryl, or an —S(O)ᵣ—CH₃ where r is 0-2, or two adjacent positions may be substituted by —O—CH₂—O— or —O—C(CH₃)₂—O—,
a monocyclic C₅-C₇-heteroaryl which may optionally be mono- or polysubstituted, identically or differently, by C₁-C₄-alkyl, provided that R² is cyano or when R¹ and/or R2 are the same or different and are each a C₁-C₄-alkyl radical, where at least one of the radicals is at least monosubstituted,
a monocyclic C₅-C₇-heteroaryl which may be at least mono- or polysubstituted, identically or differently, by halogen, CF₃, C₁-C₄-acyl, C₁-C₄-alkoxy, hydroxyl, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂,
a bi- or tricyclic C₈-C₁₂-heteroaryl which may optionally be mono- or polysubstituted, identically or differently, by halogen, by C₁-C₄-alkyl, C₁-C₄-acyl, C₁-C₄-alkoxy, hydroxyl, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂ or
a C₃-C₆-cycloalkyl which may optionally be mono- or polysubstituted, identically or differently, by halogen, by C₁-C₄-alkyl, hydroxyl, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R⁴ and R⁵ are in ortho, meta or meta,para positions with respect to one another and together are defined as —O—CO—S—, —S—CO—O—, —CH₂—CO—O—, O—CO—CH₂—, —CH₂—CO—NH—, —NH—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂— where m is 1-3,
Y is a —(CH₂)₂ group,
and the isomers, diastereomers, enantiomers and salts or cyclodextrin clathrates thereof.

5. Compounds according to claim 14, where
A is a phenyl, naphthyl or C₅-C₁₂-heteroaryl radical which may optionally be mono- or disubstituted by R⁴ and/or R⁵,
R¹ is a C₁-C₄-alkyl radical,
R² is a hydrogen, fluorine, chlorine, bromine or C₁-C₄-alkyl,
R³ is a hydrogen, methyl, ethyl, trifluoromethyl, cyano, chlorine or bromine,
R⁴ is a hydrogen, halogen, amino, an —SO(O)ₚ—C₁-C₄-alkyl group where p is 0-2,
a C₁-C₄-acyl, NH—CO—NH₂, —O—CO—NH(C₁-C₄-alkyl), —O—CO—N(C₁-C₄-alkyl)₂ or NH—CO—C₁-C₄-alkyl radical,
a C₁-C₄-alkyl which may optionally be mono- or polysubstituted, identically or differently, by C₁-C₄-acyl, C₁-C₄-alkoxy, hydroxyl, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
a C₁-C₄-alkoxy which may optionally be mono- or polysubstituted, identically or differently, by hydroxyl, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂, an O—C₆-C₁₂-aryl which may optionally be substituted by hydroxyl, cyano, COOH or CO—NH₂,
a CH₂O—C₆-C₁₂-aryl which may optionally be substituted by hydroxyl, cyano, COOH or CO—NH₂,
an O—C₅-C₁₆-heteroaryl which may optionally be substituted by hydroxyl, cyano, COOH or CO—NH₂,
a hydroxyl, cyano, O—CO—(C₁-C₄-alkyl), CO—NH(C₅-C₁₂-heteroaryl), NH—(C₁-C₄-alkyl), N—(C₁-C₄-alkyl)₂,
a C₆-C₁₂-aryl which may optionally be mono- or polysubstituted, identically or differently, by halogen, by C₁-C₄-alkyl, C₃-C₆-cycloalkyl, C₁-C₄-acyl, C₁-C₄-alkoxy, C₆-C₁₂-aryl, C₅-C₁₂-heteroaryl, hydroxyl, CH₂—OH, cyano, CH₂—CN, amino, CO₂—(C₁-C₄-alkyl), N—(C₁-C₄-alkyl)₂, NHSO₂CH₃, SO₂NH₂, SO₂NH(C₁-C₄-alkyl), SO₂N(C₁-C₄-alkyl)₂, COOH, CO—NH₂, CO—NH(C₁-C₄-alkyl), CO—N(C₁-C₄-alkyl)₂, CO—NH(C₅-C₁₂-heteroaryl), NH—CO(C₁-C₄-alkyl), CH₂—NH—CO(C₁-C₄-alkyl), NH—CO(C₅-C₁₂-heteroaryl), CH₂—NH—CO(C₅-C₁₂-heteroaryl), styryl, or by —S(O)ᵣ—CH₃ where r is 0-2, or two adjacent positions may be substituted by —O—CH₂—O—or —O—C(CH₃)₂—O—,
a monocyclic C₅-C₇-heteroaryl which may optionally be mono- or polysubstituted, identically or differently, by C₁-C₄-alkyl,
provided that R² cyano or R¹ and/or R² are the same or different and are each a C₁-C₄-alkyl radical, where at least one of the radicals is at least monosubstituted or
provided that R⁵ is an —SO(O)ₚ—C₁-C₄-alkyl where p is 0-2, a C₁-C₆-acyl, —O—CO—NH(C₁-C₄-alkyl), —O—CO—N(C₁-C₄-alkyl)₂, C₆-C₁₂-aryloxy, C₅-C₁₆-heteroaryloxy, hydroxyl, cyano or N—(C₁-C₄-alkyl)₂,
a monocyclic C₅-C₇-heteroaryl which may be at least mono- or polysubstituted, identically or differently, by halogen, CF₃, C₁-C₄-acyl, C₁-C₄-alkoxy, hydroxyl, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂,
a bi- or tricyclic C₈-C₁₂-heteroaryl which may optionally be mono- or polysubstituted, identically or differently, by halogen, by C₁-C₄-alkyl, C₁-C₄-acyl, C₁-C₄-alkoxy, hydroxyl, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂ or
a C₃-C₆-cycloalkyl which may optionally be mono- or polysubstituted, identically or differently, by halogen, by C₁-C₄-alkyl, hydroxyl, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R⁵ is a hydrogen, halogen, amino, —SO(O)ₚ—C₁-C₄-alkyl where p is 0-2,
a C₁-C₄-acyl, NH—CO—NH₂—, NH—CO—C₁-C₄-alkyl-, —O—CO—NH(C₁-C₄-alkyl)-, —O—CO—N(C₁-C₄-alkyl)₂ or C₁-C₄-alkyl group which may optionally be mono- or polysubstituted, identically or differently, by C₁-C₄-acyl, C₁-C₄-alkoxy, hydroxyl, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
a C₁-C₄-alkoxy which may optionally be mono- or polysubstituted, identically or differently, by hydroxyl, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
an O—C₆-C₁₂-aryl which may optionally be substituted by hydroxyl, cyano, COOH or CO—NH₂,
a CH₂O—C₆-C₁₂-aryl which may optionally be substituted by hydroxyl, cyano, COOH or CO—NH₂,
an O—C₅-C₁₆-heteroaryl which may optionally be substituted by hydroxyl, cyano, COOH or CO—NH₂,
a hydroxyl, cyano, O—CO—(C₁-C₄-alkyl), CO—NH(C₅-C₁₂-heteroaryl), NH—(C₁-C₄-alkyl), N—(C₁-C₄-alkyl)₂,
a C₆-C₁₂-aryl which may optionally be mono- or polysubstituted, identically or differently, by halogen, by C₁-C₄-alkyl, C₃-C₆-cycloalkyl, C₁-C₄-acyl, C₁-C₄-alkoxy, C₆-C₁₂-aryl, C₅-C₁₂-heteroaryl, hydroxyl, CH₂—OH, cyano, CH₂—CN, amino, CO₂—(C₁-C₄-alkyl), N—(C₁-C₄-alkyl)₂, NHSO₂CH₃, SO₂NH₂, SO₂NH(C₁-C₄-alkyl), SO₂N(C₁-C₄-alkyl)₂, COOH, CO—NH₂, CO—NH(C₁-C₄-alkyl), CO—N(C₁-C₄-alkyl)₂, CO—NH(C₅-C₁₂-heteroaryl), NH—CO(C₁-C₄-alkyl), CH₂—NH—CO(C₁-C₄-alkyl), NH—CO(C₅-C₁₂-heteroaryl), CH₂—NH—CO(C₅-C₁₂-heteroaryl), styryl, or an —S(O)ᵣ—CH₃ where r is 0-2, or two adjacent positions may be substituted by —O—CH₂—O— or —O—C(CH₃)₂—O—,
a monocyclic C₅-C₇-heteroaryl which may optionally be mono- or polysubstituted, identically or differently, by C₁-C₄-alkyl, provided that R² is cyano or when R¹ and/or R² are the same or different and are each a C₁-C₄-alkyl radical, where at least one of the radicals is at least monosubstituted,
a monocyclic C₅-C₇-heteroaryl which may be at least mono- or polysubstituted, identically or differently, by halogen, CF₃, C₁-C₄-acyl, C₁-C₄-alkoxy, hydroxyl, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂,
a bi- or tricyclic C₈-C₁₂-heteroaryl which may optionally be mono- or polysubstituted, identically or differently, by halogen, by C₁-C₄-alkyl, C₁-C₄-acyl, C₁-C₄-alkoxy, hydroxyl, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂ or
a C₃-C₆-cycloalkyl which may optionally be mono- or polysubstituted, identically or differently, by halogen, by C₁-C₄-alkyl, hydroxyl, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R⁴ and R⁵ are in ortho, meta or meta,para positions with respect to one another and together are defined as —O—CO—S—, —S—CO—O—, CH₂—CO—O—, O—CO—CH₂—, —CH₂—CO—NH—, —NH—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
Y is a —(CH₂)₂ group,
and the isomers, diastereomers, enantiomers and salts or cyclodextrin clathrates thereof.

6. Compounds according to claim 1, where
A is a phenyl, naphthyl or C₅-C₁₂-heteroaryl radical which may optionally be mono- or disubstituted by R⁴ and/or R⁵,
R¹ is a C₁-C₄-alkyl radical,
R² is a hydrogen, fluorine, chlorine, bromine or C₁-C₄-alkyl,
R³ is a hydrogen, methyl, ethyl, trifluoromethyl, cyano, chlorine or bromine,
R⁴ is a hydrogen, halogen, amino, an —SO(O)ₚ—C₁-C₄-alkyl group where p is 0-2,
a C₁-C₄-acyl, NH—CO—NH₂, —O—CO—NH(C₁-C₄-alkyl), —O—CO—N(C₁-C₄-alkyl)₂ or NH—CO—C₁-C₄-alkyl radical,
a C₁-C₄-alkyl which may optionally be mono-, di- or trisubstituted, identically or differently, by C₁-C₄-acyl, C₁-C₄-alkoxy, hydroxyl, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
a C₁-C₄-alkoxy which may optionally be mono-, di- or trisubstituted, identically or differently, by hydroxyl, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
an O—C₆-C₁₂-aryl which may optionally be substituted by hydroxyl, cyano, COOH or CO—NH₂,
a CH₂O—C₆-C₁₂-aryl which may optionally be substituted by hydroxyl, cyano, COOH or CO—NH₂,
an O—C₅-C₁₆-heteroaryl which may optionally be substituted by hydroxyl, cyano, COOH or CO—NH₂,
a hydroxyl, cyano, O—CO—(C₁-C₄-alkyl), CO—NH(C₅-C₁₂-heteroaryl), NH—(C₁-C₄-alkyl), N—(C₁-C₄-alkyl)₂,
a C₆-C₁₂-aryl which may optionally be mono-, di- or trisubstituted, identically or differently, by halogen, by C₁-C₄-alkyl, C₃-C₆-cycloalkyl, C₁-C₄-acyl, C₁-C₄-alkoxy, C₆-C₁₂-aryl, C₅-C₁₂-heteroaryl, hydroxyl, CH₂—OH, cyano, CH₂—CN, amino, CO₂—(C₁-C₄-alkyl), N—(C₁-C₄-alkyl)₂, NHSO₂CH₃, SO₂NH₂, SO₂NH(C₁-C₄-alkyl), SO₂N(C₁-C₄-alkyl)₂, COOH, CO—NH₂, CO—NH(C₁-C₄-alkyl), CO—N(C₁-C₄-alkyl)₂, CO—NH(C₅-C₁₂-heteroaryl), NH—CO(C₁-C₄-alkyl), CH₂—NH—CO(C₁-C₄-alkyl), NH—CO(C₅-C₁₂-heteroaryl), CH₂—NH—CO(C₅-C₁₂-heteroaryl), styryl, or by —S(O)ᵣ—CH₃ where r is 0-2, or two adjacent positions may be substituted by —O—CH₂—O— or —O—C(CH₃)₂—O—,
a monocyclic C₅-C₇-heteroaryl which may optionally be mono-, di- or trisubstituted, identically or differently, by C₁-C₄-alkyl,
provided that R² is cyano or when R¹ and/or R² are the same or different and are each a C₁-C₄-alkyl radical, where at least one of the radicals is at least monosubstituted or
provided that R⁵ is an —SO(O)ₚ—C₁-C₄-alkyl where p is 0-2, is a C₁-C₆-acyl, —O—CO—NH(C₁-C₄-alkyl), —O—CO—N(C₁-C₄-alkyl)₂, C₆-C₁₂-aryloxy, C₅-C₁₆-heteroaryloxy, hydroxyl, cyano or N—(C₁-C₄-alkyl)₂,
a monocyclic C₅-C₇-heteroaryl which may be at least mono-, di- or trisubstituted, identically or differently, by halogen, CF₃, C₁-C₄-acyl, C₁-C₄-alkoxy, hydroxyl, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂,
a bi- or tricyclic C₈-C₁₂-heteroaryl which may optionally be mono-, di- or trisubstituted, identically or differently, by halogen, by C₁-C₄-alkyl, C₁-C₄-acyl, C₁-C₄-alkoxy, hydroxyl, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂ or
a C₃-C₆-cycloalkyl which may optionally be mono-, di- or trisubstituted, identically or differently, by halogen, by C₁-C₄-alkyl, hydroxyl, cyano, CO₂—(C₁-C₄-alkyl), C₁-C₄-acyl, N—(C₁-C₄-alkyl)₂, COOH, CO—NH₂, CO—NH(C₁-C₄-alkyl), CO—N(C₁-C₄-alkyl)₂ or C₁-C₄-alkoxy,
R⁵ is a hydrogen, halogen, amino, —SO(O)ₚ—C₁-C₄-alkyl where p is 0-2,
a C₁-C₄-acyl, NH—CO—NH₂, NH—CO—C₁-C₄-alkyl, —O—CO—NH(C_{1-C}₄-alkyl), —O—CO—N(C₁-C₄-alkyl)₂ or C₁-C₄-alkyl group which may optionally be mono-, di- or trisubstituted, identically or differently, by C₁-C₄-acyl, C₁-C₄-alkoxy, hydroxyl, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
a C₁-C₄-alkoxy which may optionally be mono-, di- or trisubstituted, identically or differently, by hydroxyl, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
an O—C₆-C₁₂-aryl which may optionally be substituted by hydroxyl, cyano, COOH or CO—NH₂,
a CH₂O—C₆-C₁₂-aryl which may optionally be substituted by hydroxyl, cyano, COOH or CO—NH₂,
an O—C₅-C₁₆-heteroaryl which may optionally be substituted by hydroxyl, cyano, COOH or CO—NH₂,
a hydroxyl, cyano, O—CO—(C₁-C₄-alkyl), CO—NH(C₅-C₁₂-heteroaryl), NH—(C₁-C₄-alkyl), N—(C₁-C₄-alkyl)₂,
a C₆-C₁₂-aryl which may optionally be mono-, di- or trisubstituted, identically or differently, by halogen, by C₁-C₄-alkyl, C₃-C₆-cycloalkyl, C₁-C₄-acyl, C₁-C₄-alkoxy, C₆-C₁₂-aryl, C₅-C₁₂-heteroaryl, hydroxyl, CH₂—OH, cyano, CH₂—CN, amino, CO₂—(C₁-C₄-alkyl), N—(C₁-C₄-alkyl)₂, NHSO₂CH₃, SO₂NH₂, SO₂NH(C₁-C₄-alkyl), SO₂N(C₁-C₄-alkyl)₂, COOH, CO—NH₂, CO—NH(C₁-C₄-alkyl), CO—N(C₁-C₄-alkyl)₂, CO—NH(C₅-C₁₂-heteroaryl), NH—CO(C₁-C₄-alkyl), CH₂—NH—CO(C₁-C₄-alkyl), NH—CO(C₅-C₁₂-heteroaryl), CH₂—NH—CO(C₅-C₁₂-heteroaryl), styryl, or by —S(O)ᵣ—CH₃ where r is 0-2, or two adjacent positions may be substituted by —O—CH₂—O— or —O—C(CH₃)₂—O—,
a monocyclic C₅-C₇-heteroaryl which may optionally be mono-, di- or trisubstituted, identically or differently, by C₁-C₄-alkyl,
provided that R² is cyano or when R¹ and/or R² are the same or different and are each a C₁-C₄-alkyl radical, where at least one of the radicals is at least monosubstituted,
a monocyclic C₅-C₇-heteroaryl which may be at least mono-, di- or trisubstituted, identically or differently, by halogen, CF₃, C₁-C₄-acyl, C₁-C₄-alkoxy, hydroxyl, CH₂—OH, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂,
a bi- or tricyclic C₈-C₁₂-heteroaryl which may optionally be mono-, di- or trisubstituted, identically or differently, by halogen, by C₁-C₄-alkyl, C₁-C₄-acyl, C₁-C₄-alkoxy, hydroxyl, cyano, CO₂—(C₁-C₄-alkyl), N—(C₁-C₄-alkyl)₂, COOH, CO—NH₂, CO—NH(C₁-C₄-alkyl) or CO—N(C₁-C₄-alkyl)₂ or
a C₃-C₆-cycloalkyl which may optionally be mono-, di- or trisubstituted, identically or differently, by halogen, by C₁-C₄-alkyl, hydroxyl, cyano, CO₂—(C₁-C₄-alkyl), C₁-C₄-acyl, N—(C₁-C₄-alkyl)₂, COOH, CO—NH₂, CO—NH(C_{1-C}₄-alkyl), CO—N(C₁-C₄-alkyl)₂ or C₁-C₄-alkoxy,
R⁴ and R⁵ are in ortho, meta or meta,para positions with respect to one another and together are defined as —O—CO—S—, —S—CO—O—, CH₂—CO—O—, O—CO—CH₂—, —CH₂—CO—NH—, —NH—CO—CH₂—, —O—CO—NH—, —NH—CO—O—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
Y is a —(CH₂)₂ group,
and the isomers, diastereomers, enantiomers and salts or cyclodextrin clathrates thereof.

7. Compounds according to claim 1, where
A is a phenyl, naphthyl or C₅-C₁₂-heteroaryl radical which may optionally be mono- or disubstituted by R⁴ and/or R⁵,
R¹ is a C₁-C₄-alkyl radical,
R² is a hydrogen, fluorine, chlorine, bromine, methyl, ethyl or trifluoromethyl,
R³ is a hydrogen, methyl, ethyl, trifluoromethyl, cyano, chlorine or bromine,
R⁴ is a hydrogen, halogen, amino,
a C₁-C₄-acyl or NH—CO—C₁-C₄-alkyl radical,
a C₁-C₄-alkyl which may optionally be mono- or disubstituted, identically or differently, by C₁-C₄-acyl, C₁-C₄-alkoxy, hydroxyl, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
a C₁-C₄-alkoxy which may optionally be monosubstituted by hydroxyl, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
an O-phenyl which may optionally be mono- or disubstituted by hydroxyl, cyano, COOH or CO—NH₂,
a CH₂O-phenyl which may optionally be mono- or disubstituted by hydroxyl, cyano, COOH or CO—NH₂,
an O—C₅-C₇-heteroaryl which may optionally be mono- or disubstituted by hydroxyl, cyano, COOH or CO—NH₂,
a hydroxyl, cyano, O—CO—(C₁-C₄-alkyl), CO—NH(C₅-C₁₂-heteroaryl), NH—(C₁-C₄-alkyl), N—(C₁-C₄-alkyl)₂,
a phenyl which may optionally be mono- or disubstituted, identically or differently, by halogen, by C₁-C₄-alkyl, C₁-C₄-acyl, C₁-C₄-alkoxy, hydroxyl, CH₂—OH, cyano, CH₂—CN, amino, CO₂—(C₁-C₄-alkyl), N—(C₁-C₄-alkyl)₂, NHSO₂CH₃, SO₂NH₂, SO₂NH(C₁-C₄-alkyl), SO₂N(C₁-C₄-alkyl)₂, COOH, CO—NH₂, CO—NH(C₁-C₄-alkyl), CO—N(C₁-C₄-alkyl)₂, NH—CO(C₁-C₄-alkyl), CH₂—NH—CO(C₁-C₄-alkyl), or two adjacent positions may be substituted by —O—CH₂—O— or —O—C(CH₃)₂—O—,
R⁵ is a hydrogen, halogen, amino,
a C₁-C₄-acyl or NH—CO—C₁-C₄-alkyl radical,
a C₁-C₄-alkyl which may optionally be mono- or disubstituted, identically or differently, by C₁-C₄-acyl, C₁-C₄-alkoxy, hydroxyl, cyano, CO₂—(C₁-C₄-alkyl), N—(C₁-C₄-alkyl)₂, C₅-C₁₂-heteroaryl, COOH, CO—NH₂, CO—NH(C₁-C₄-alkyl) or by CO—N(C₁-C₄-alkyl)₂,
a C₁-C₄-alkoxy which may optionally be monosubstituted by hydroxyl, cyano, CO₂—(C₁-C₄-alkyl), N—(C₁-C₄-alkyl)₂, NH—C₃-C₆-cycloalkyl, COOH, CO—NH₂, CO—NH(C₁-C₄-alkyl) or by CO—N(C₁-C₄-alkyl)₂,
an O-phenyl which may optionally be mono- or disubstituted by hydroxyl, cyano, COOH or CO—NH₂,
a CH₂O-phenyl which may optionally be mono- or disubstituted by hydroxyl, cyano, COOH or CO—NH₂,
an O—C₅-C₇-heteroaryl which may optionally be mono- or disubstituted by hydroxyl, cyano, COOH or CO—NH₂,
a hydroxyl, cyano, O—CO—(C₁-C₄-alkyl), CO—NH(C₅-C₁₂-heteroaryl), NH—(C₁-C₄-alkyl), N—(C₁-C₄-alkyl)₂,
a phenyl which may optionally be mono- or disubstituted, identically or differently, by halogen, by C₁-C₄-alkyl, C₁-C₄-acyl, C₁-C₄-alkoxy, hydroxyl, CH₂—OH, cyano, CH₂—CN, amino, CO₂—(C₁-C₄-alkyl), N—(C₁-C₄-alkyl)₂, NHSO₂CH₃, SO₂NH₂, SO₂NH(C₁-C₄-alkyl), SO₂N(C₁-C₄-alkyl)₂, COOH, CO—NH₂, CO—NH(C₁-C₄-alkyl), CO—N(C₁-C₄-alkyl)₂, NH—CO(C₁-C₄-alkyl), CH₂—NH—CO(C₁-C₄-alkyl), or two adjacent positions may be substituted by —O—CH₂—O— or —O—C(CH₃)₂—O—,
R⁴ and R⁵ are in ortho, meta or meta,para positions with respect to one another and together are defined as CH₂—CO—NH—, —NH—CO—CH₂—, —CO—CH₂—(CH₂)ₘ—, —CH₂—(CH₂)ₘ—CO—, —O—(CH₂)ₘ—O—, —O—C—(CH₃)₂—O—, —CH₂—(CH₂)ₘ—CH₂—, where m is 1-3,
Y is a —(CH₂)₂ group,
and the isomers, diastereomers, enantiomers and salts or cyclodextrin clathrates thereof.

8. Compounds according to claim 1, selected from a group comprising the following compounds:
1. 3,4-dimethoxy-N-[2-(2-methyl-1H-indol-3-yl)ethyl]benzamide
2. N-[2-(7-chloro-2-methyl-1H-indol-3-yl)ethyl]-5-fluoro-1H-indole-2-carboxamide
3. N-[2-(7-chloro-4-fluoro-2-methyl-1H-indol-3-yl)ethyl]-5-fluoro-1H-indole-2-carboxamide
4. 3-N-methyl-4′-N′-[2-(7-chloro-4-fluoro-2-methyl-1H-indol-3-yl)ethyl]-biphenyl-3,4′-dicarboxamide
5. N-[2-(7-chloro-2-methyl-1H-indol-3-yl)ethyl]-6-(3-methylcarbamoyl-phenyl)nicotinamide
6. N-[2-(7-chloro-2-methyl-1H-indol-3-yl)ethyl]-3′-hydroxybiphenyl-4-carboxamide
7. N-[2-(7-chloro-4-fluoro-2-methyl-1H-indol-3-yl)ethyl]-3,4-dimethoxy-benzamide
8. 3-N-methyl-4′-N′-[2-(2-methyl-1H-indol-3-yl)ethyl]biphenyl-3,4′-dicarboxamide
9. N-[2-(2-methyl-1H-indol-3-yl)ethyl]-5-fluoro-1H-indole-2-carboxamide
10. N-[2-(2-methyl-1H-indol-3-yl)ethyl]-5-chloro-1H-indole-2-carboxamide
11. N-[2-(2,4-dimethyl-1H-indol-3-yl)ethyl]-3,4-dimethoxybenzamide
12. N-[2-(7-chloro-2,4-dimethyl-1H-indol-3-yl)ethyl]-3,4-dimethoxybenzamide
13. N-[2-(7-bromo-2,4-dimethyl-1H-indol-3-yl)ethyl]-3,4-dimethoxybenzamide
14. N-[2-(7-bromo-2,4-dimethyl-1H-indol-3-yl)ethyl]-3H-benzotriazole-5-carboxamide
15. N-[2-(7-bromo-2,4-dimethyl-1H-indol-3-yl)ethyl]-1H-indole-2-carboxamide
16. N-[2-(7-bromo-2,4-dimethyl-1H-indol-3-yl)ethyl]quinoxaline-6-carboxamide
17. N-[2-(7-chloro-2,4-dimethyl-1H-indol-3-yl)ethyl]-3H-benzotriazole-5-carboxamide
18. N-[2-(7-chloro-2,4-dimethyl-1H-indol-3-yl)ethyl]quinoxaline-6-carboxamide
19. N-[2-(7-chloro-2,4-dimethyl-1H-indol-3-yl)ethyl]-1H-indole-2-carboxamide
20. N-[2-(7-bromo-2-methyl-1H-indol-3-yl)ethyl]-3,4-dimethoxybenzamide
21. 3-N-methyl-4′-N′-[2-(7-bromo-2-methyl-1H-indol-3-yl)ethyl]biphenyl-3,4′-dicarboxamide
22. N-[2-(7-bromo-2-methyl-1H-indol-3-yl)ethyl]-5-fluoro-1H-indole-2-carboxamide
23. N-[2-(7-chloro-2-trifluoromethyl-1H-indol-3-yl)ethyl]-3,4-dimethoxy-benzamide
24. N-[2-(7-chloro-2-trifluoromethyl-1H-indol-3-yl)ethyl]-5-fluoro-1H-indole-2-carboxamide
25. N-[2-(7-chloro-2-trifluoromethyl-1H-indol-3-yl)ethyl]-5-chloro-1H-indole-2-carboxamide
26. 3-N-methyl-4′-N′-[2-(7-chloro-2-trifluoromethyl-1H-indol-3-yl)ethyl]biphenyl-3,4′-dicarboxamide
27. N-[2-(7-chloro-2-trifluoromethyl-1H-indol-3-yl)ethyl]-5-trifluoromethoxy-1H-indol-2-carboxamide
28. N-[2-(7-chloro-2-trifluoromethyl-1H-indol-3-yl)ethyl]benzofuran-2-carboxamide
29. N-[2-(7-chloro-2-trifluoromethyl-1H-indol-3-yl)ethyl]benzo[b]thiophene-2-carboxamide
30. N-[2-(7-bromo-2-methyl-1H-indol-3-yl)ethyl]-5-chloro-1H-indole-2-carboxamide
31. N-[2-(7-chloro-2-methyl-1H-indol-3-yl)ethyl]-5-chloro-1H-indole-2-carboxamide
32. N-[2-(2-tert-butyl-1H-indol-3-yl)ethyl]-3,4-dimethoxybenzamide
33. N-[2-(2-tert-butyl-1H-indol-3-yl)ethyl]-5-trifluoromethoxy-1H-indole-2-carboxamide
34. N-[2-(2-tert-butyl-1H-indol-3-yl)ethyl]-5-chloro-1H-indole-2-carboxamide
35. N-[2-(2-tert-butyl-1H-indol-3-yl)ethyl]-5-trifluoromethyl-1H-indole-2-carboxamide
36. N-[2-(2-tert-butyl-1H-indol-3-yl)ethyl]-5-bromo-1H-indole-2-carboxamide
37. N-[2-(2-tert-butyl-1H-indol-3-yl)ethyl]-5-fluoro-1H-indole-2-carboxamide
38. 2-bromo-N-[2-(2,7-dimethyl-1H-indol-3-yl)ethyl]-4,5-dimethoxybenzamide
39. N-[2-(2,7-dimethyl-1H-indol-3-yl)ethyl]-1H-indole-6-carboxamide
40. N-[2-(2,7-dimethyl-1H-indol-3-yl)ethyl]quinoline-5-carboxamide
41. N-[2-(2,7-dimethyl-1H-indol-3-yl)ethyl]-5-phenyl-1H-pyrazole-4-carboxamide
42. N-[2-(2,7-dimethyl-1H-indol-3-yl)ethyl]pyridazine-4-carboxamide
43. N-[2-(2,7-dimethyl-1H-indol-3-yl)ethyl]-5-phenyl-2H-pyrazole-3-carboxamide
44. N-[2-(2,7-dimethyl-1H-indol-3-yl)ethyl]pyrimidine-5-carboxamide
45. N-[2-(2,7-dimethyl-1H-indol-3-yl)ethyl]pyrazolo[1,5-a]pyridine-2-carboxamide
46. N-[2-(2,7-dimethyl-1H-indol-3-yl)ethyl]benzo[b]thiophene-5-carboxamide
47. N-[2-(2,7-dimethyl-1H-indol-3-yl)ethyl]quinoxaline-2-carboxamide
48. N-[2-(2,7-dimethyl-1H-indol-3-yl)ethyl]-3-fluoro-2-methoxybenzamide
49. 3-chloro-N-[2-(2,7-dimethyl-1H-indol-3-yl)ethyl]-2-methylbenzamide
50. 3-chloro-N-[2-(2,7-dimethyl-1H-indol-3-yl)ethyl]-2-fluorobenzamide
51. N-[2-(2,7-dimethyl-1H-indol-3-yl)ethyl]-4-phenoxybenzamide
52. N-[2-(2,7-dimethyl-1H-indol-3-yl)ethyl]thiazole-4-carboxamide
53. 2-chloro-N-[2-(2,7-dimethyl-1H-indol-3-yl)ethyl]-3-methylbenzamide
54. N-[2-(2,7-dimethyl-1H-indol-3-yl)ethyl]quinoline-4-carboxamide
55. N-[2-(2,7-dimethyl-1H-indol-3-yl)ethyl]-3-hydroxybenzamide
56. N-[2-(2,7-dimethyl-1H-indol-3-yl)ethyl]-4-hydroxy-2-phenyl-2H-pyrazole-3-carboxamide
57. N-[2-(2,7-dimethyl-1H-indol-3-yl)ethyl]benzo[b]thiophene-3-carboxamide
58. N-[2-(2,7-dimethyl-1H-indol-3-yl)ethyl]-1H-indole-2-carboxamide
59. N-[2-(2,7-dimethyl-1H-indol-3-yl)ethyl]quinoline-2-carboxamide
60. N-[2-(2,7-dimethyl-1H-indol-3-yl)ethyl]furan-2-carboxamide
61. 2-chloro-N-[2-(2,7-dimethyl-1H-indol-3-yl)ethyl]nicotinamide
62. N-[2-(2,7-dimethyl-1H-indol-3-yl)ethyl]quinoline-3-carboxamide
63. 5-bromo-N-[2-(2,7-dimethyl-1H-indol-3-yl)ethyl]nicotinamide
64. N-[2-(2,7-dimethyl-1H-indol-3-yl)ethyl]-1H-indole-3-carboxamide
65. 4-benzyloxy-N-[2-(2,7-dimethyl-1H-indol-3-yl)ethyl]benzamide
66. N-[2-(2,7-dimethyl-1H-indol-3-yl)ethyl]-3-bromothiophene-2-carboxamide
67. N-[2-(2,7-dimethyl-1H-indol-3-yl)ethyl]-2-methylfuran-3-carboxamide
68. N-[2-(2,7-dimethyl-1H-indol-3-yl)ethyl]-4-oxo-4,5,6,7-tetrahydro-benzo[b]thiophene-2-carboxamide
69. 2-benzo[1,2,5]thiadiazol-4-yl-N-[2-(2,7-dimethyl-1H-indol-3-yl)ethyl]-acetamide
70. N-[2-(2,7-dimethyl-1H-indol-3-yl)ethyl]-1H-imidazole-4-carboxamide
71. N-[2-(2,7-dimethyl-1H-indol-3-yl)ethyl]-4′-bromobiphenyl-2-carboxamide
72. N-[2-(2,7-dimethyl-1H-indol-3-yl)ethyl]-2-fluoro-6-iodobenzamide
73. 3′-[2-(2,7-dimethyl-1H-indol-3-yl)ethylcarbamoyl]biphenyl-2-carboxylic acid methyl ester
74. N-[2-(2,7-dimethyl-1H-indol-3-yl)ethyl]-2,3-dihydrobenzofuran-7-carboxamide
75. N-[2-(2,7-dimethyl-1H-indol-3-yl)ethyl]-3-fluoro-2-methylbenzamide
76. N-[2-(2,7-dimethyl-1H-indol-3-yl)ethyl]-2,5-dimethylbenzamide
77. N-[2-(2,7-dimethyl-1H-indol-3-yl)ethyl]-5-acetylthiophene-2-carboxamide
78. N-[2-(2,7-dimethyl-1H-indol-3-yl)ethyl]-2-fluoro-3-methoxybenzamide
79. N-[2-(2,7-dimethyl-1H-indol-3-yl)ethyl]quinoline-8-carboxamide
80. N-[2-(2,7-dimethyl-1H-indol-3-yl)ethyl]-4-methylsulfanylbenzamide
81. N-[2-(2,7-dimethyl-1H-indol-3-yl)ethyl]-2-phenyl-2H-pyrazole-3-carboxamide
82. N-[2-(2,7-dimethyl-1H-indol-3-yl)ethyl]-6-phenylpyrimidine-4-carboxamide
83. N-[2-(2,7-dimethyl-1H-indol-3-yl)ethyl]-1l-methyl-i H-indole-3-carboxamide
84. N-[2-(2,7-dimethyl-1H-indol-3-yl)ethyl]-2-phenoxymethylbenzamide
85. N-[2-(2,7-dimethyl-1H-indol-3-yl)ethyl]-2,5-dimethyl-2H-pyrazole-3-carboxamide
86. N-[2-(2,7-dimethyl-1H-indol-3-yl)ethyl]-2,3-dihydrobenzofuran-5-carboxamide
87. N-[2-(2,7-dimethyl-1H-indol-3-yl)ethyl]-5-methoxy-1H-indole-2-carboxamide
88. N-[2-(2-methyl-7-trifluoromethyl-1H-indol-3-yl)ethyl]-5-methoxy-1H-indole-2-carboxamide
89. N-[2-(2-methyl-7-trifluoromethyl-1H-indol-3-yl)ethyl]-1H-indole-2-carboxamide
90. N-[2-(2-methyl-7-trifluoromethyl-1H-indol-3-yl)ethyl]-5-fluoro-1H-indole-2-carboxamide
91. N-[2-(2-methyl-7-trifluoromethyl-1H-indol-3-yl)ethyl]-5-methyl-1H-indole-2-carboxamide
92. N-[2-(2-methyl-7-trifluoromethyl-1H-indol-3-yl)ethyl]-6-methoxy-1H-indole-2-carboxamide
93. N-[2-(2-methyl-7-trifluoromethyl-1H-indol-3-yl)ethyl]-4-methyl-1H-indole-2-carboxamide
94. N-[2-(2-methyl-7-trifluoromethyl-1H-indol-3-yl)ethyl]-4-methoxy-1H-indole-2-carboxamide
95. N-[2-(2-methyl-7-trifluoromethyl-1H-indol-3-yl)ethyl]-4-fluoro-1H-indole-2-carboxamide
96. N-[2-(2-methyl-7-trifluoromethyl-1H-indol-3-yl)ethyl]-6-fluoro-1H-indole-2-carboxamide
97. N-[2-(2-methyl-7-trifluoromethyl-1H-indol-3-yl)ethyl]-6-methyl-1H-indole-2-carboxamide
98. N-[2-(2-methyl-7-trifluoromethyl-1H-indol-3-yl)ethyl]-7-methyl-1H-indole-2-carboxamide
99. N-[2-(7-cyano-2,4-dimethyl-1H-indol-3-yl)ethyl]-3,4-dimethoxybenzamide
100. N-[2-(7-chloro-2-methyl-1H-indol-3-yl)ethyl]-3,4-dimethoxybenzamide
101. 4-N-methyl-4′-N′-[2-(7-chloro-2-methyl-1H-indol-3-yl)ethyl]biphenyl-4,4′-dicarboxamide
102. 3-N-methyl-4′-N′-[2-(7-chloro-2-methyl-1H-indol-3-yl)ethyl]biphenyl-3,4′-dicarboxamide
103. N-[2-(7-bromo-2,4-dimethyl-1H-indol-3-yl)ethyl]-3,4-dimethoxybenzamide
104. N-[2-(4,7-dichloro-2-methyl-1H-indol-3-yl)ethyl]-3,4-dimethoxybenzamide
105. 3-N-methyl-4′-N′-[2-(4,7-dichloro-2-methyl-1H-indol-3-yl)ethyl]biphenyl-3,4′-dicarboxamide

9. Use of the compounds of the formula I for the production of a medicament.

10. Use of the medicament according to claim 9, characterized in that the medicament is used for treatment and prophylaxis of disorders.

11. Use of the medicament according to claim 9 for treatment and prophylaxis of disorders associated with the EP₂ receptor.

12. Use of the medicament according to claim 9 for treatment and prophylaxis of fertility disorders.

13. Use of the medicament according to claim 9 for treatment and prophylaxis of menstrual complaints, which may include heavy and long-lasting bleeding.

14. Use of the medicament according to claim 9 for treatment and prophylaxis of endometriosis.

15. Use of the medicament according to claim 9 for treatment and prophylaxis of pain.

16. Use of the compounds according to claim 1 for fertility control/contraception.

17. Use of the medicament according to claim 9 for treatment and prophylaxis of osteoporosis.

18. Use of the medicament according to claim 9 for treatment and prophylaxis of cancer.

19. Use of the medicament according to claim 9 for treatment and prophylaxis of Alzheimer's disease.

20. Use of the medicament according to claim 9 for treatment and prophylaxis of Parkinson's disease.

21. Use of the medicament according to claim 9 for treatment and prophylaxis of inflammatory bowel disorders, which may include Crohn's disease and ulcerative colitis.

22. Use of the medicament according to claim 9 for treatment and prophylaxis of polycystic kidney disorders.

23. Use of the medicament according to claim 9 for treatment and prophylaxis of artherosclerosis.

24. Medicaments comprising a compound of the general formula (I) in combination with a COX inhibitor for treatment of disorders, said COX inhibitors being, for example, aspirin, naproxen, indomethacin, meloxicam, celecoxib (4-[5-(4-methylphenyl)-3-(trifluoromethyl)-1H-pyrazol-1-yl]benzenesulphonamide), ibuprofen, parecoxib (N-[4-(5-methyl-3-phenyl-4-isoxazolyl)phenyl]sulphonylpropionamide), rofecoxib (4-(4-mesylphenyl)-3-phenylfuran-2(5H)-one), valdecoxib (4-[5-methyl-3-phenyl-4-isoxazoyl)benzenesulphonamide), NS-398 (N-methyl-2-cyclohexanoxy-4-nitrobenzenesulphonamide), lumiracoxib [2-(2′-chloro-6′-fluorophenyl)-amino-5-methylbenzeneacetic, ceracoxib and etoricoxib.

25. Medicament according to claim 24, for which the disorders may be fertility disorders, menstrual complaints, endometriosis, pain, osteoporosis, cancer, Alzheimer's disease, Parkinson's disease, inflammatory bowel disorders, polycystic kidney disorders, arteriosclerosis, or which can be used for fertility control.

26. Use of the medicament according to claim 8 for treatment and prophylaxis of infections of the respiratory pathway.

27. Use of the compounds of the general formula I, according to claim 1, in the form of a pharmaceutical preparation for enteral, parenteral, vaginal and oral administration.

28. Process for preparing the compounds of the general formula (I), characterized in that a compound of the formula II 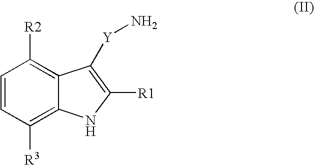
in which R¹, R², R³ and Y are each as defined in claim 1 are reacted with an acid of the general formula III 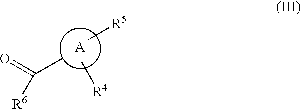
in which A, R⁴ and R⁵ are each as defined in claim 1 and R⁶ may be a hydroxyl group, a chlorine or bromine atom or a C₁-C₆-alkyl radical, preferably hydrogen, chlorine, the methyl or ethyl radical, and/or any protecting groups required are then detached and/or any double bonds present are hydrogenated and/or a bromide is substituted for a cyanide, to give the compounds of the general formula (I).
